(19) United States
(12) Patent Application Publication
(10) Pub. No.: US 2025/0339061 A1
(43) Pub. Date: Nov. 6, 2025

(54) SYSTEMS, DEVICES, AND METHODS FOR SKIN ADHESIVE LINER REMOVAL

(71) Applicant: ABBOTT DIABETES CARE INC., Alameda, CA (US)

(72) Inventors: Sean P. Day, Los Altos, CA (US); Sachin M. Shenoi, Cupertino, CA (US); Vivek S. Rao, Alameda, CA (US); Steven T. Mitchell, Pleasant Hill, CA (US); Stephen T. Pudjijanto, San Ramon, CA (US)

(21) Appl. No.: 19/196,018

(22) Filed: May 1, 2025

Related U.S. Application Data
(60) Provisional application No. 63/642,238, filed on May 3, 2024.
(60) Provisional application No. 63/654,822, filed on May 31, 2024.

(51) Int. Cl. A61B 5/145; A61B 5/00

(52) U.S. Cl. CPC A61B 5/14532; A61B 5/68335; A61B 5/0004; A61B 2560/063; A61B 2562/08

(57) ABSTRACT

An assembly for insertion of an analyte sensor is provided. The assembly can include an applicator having an applicator housing, a sharp, a carrier configured to releasably retain a sensor control device, and an applicator cap removably coupled with the applicator housing. The assembly can further include a sensor control device configured to be worn on skin of a user, which includes a sensor control device housing, sensor electronics disposed in the sensor control device housing, the analyte sensor, a sensor cap, a collar configured to snap-fit into the sensor cap, an adhesive patch comprising a distally-facing surface configured to adhere to the skin of the user, and an adhesive liner. The adhesive liner can be configured such that removal of the applicator cap and/or the sensor cap causes removal of the adhesive liner from the adhesive patch.

### CROSS-REFERENCE TO RELATED APPLICATIONS

This application claims the benefit of U.S. Provisional Application Ser. No. 63/654,822, filed May 31, and U.S. Provisional Application Ser. No. 63/642,238, filed May 3, 2024, 2024, both of which are hereby expressly incorporated by reference in their entireties for all purposes.

### FIELD

The subject matter described herein relates generally to systems, devices, and methods for removal of skin adhesive liners designed for use with a sensor control device.

### BACKGROUND

The detection and/or monitoring of analyte levels, such as glucose, ketones, lactate, oxygen, hemoglobin AIC, or the like, can be vitally important to the overall health of a person, particularly for an individual having diabetes. Patients suffering from diabetes mellitus can experience complications including loss of consciousness, cardiovascular disease, retinopathy, neuropathy, and nephropathy. Persons with diabetes are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual that requires analyte monitoring. To increase comfort and convenience for the individual, the sensor control device may have a small form-factor and can be applied by the individual with a sensor applicator. The application process includes inserting a sensor that senses a user's in vivo analyte level, using an applicator, such that the sensor comes into contact with a bodily fluid. The sensor control device may also be configured to transmit analyte data to another device, from which the individual or her health care provider (“HCP”) can review the data and make therapy decisions. Furthermore, the benefits of analyte monitoring systems are not limited to persons with diabetes. For instance, analyte monitoring systems can provide useful information and insights to individuals interested in improving their health and wellness. As one example, to improve their sports performance, athletes can utilize a sensor control device worn on the body to collect data relating to one or more analytes such as, for example, glucose and/or lactate.

Despite their desirability, one issue associated with in vivo analyte monitoring systems is that an adhesive holding the sensor control device on the surface of the skin may fail before the sensor has reached the end of its usable life. For example, perspiration, bathing, dirt accumulation, oil secretion, and the like may lead to adhesive failure and/or decoupling of the sensor control device from the individual's body. Moreover, under certain conditions within the housing and cap of the applicator, chemical interactions between the adhesive patch, trapped atmosphere, desiccant, and outgassing of materials can also cause the adhesive to degrade during storage or shipment. To mitigate these concerns, skin adhesive liners are typically applied to the adhesive patch to protect the adhesives during manufacturing. It is believed that removing the skin adhesive liner during the manufacturing process may lead to a reduction in the lifespan of the adhesives. Therefore, it is essential to delay removing the skin adhesive liners until the point of use, which occurs just before attaching the sensor control device to a user's skin.

As such, there is a need for systems, devices, and methods that can securely maintain both the liner and adhesive in position throughout manufacturing, storage, and transportation stages. Moreover, there is a need to develop efficient methods for promptly removing the liner immediately prior to utilization, ensuring optimal functionality and longevity of the adhesive patches for attaching sensor control devices to a user's skin.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter is directed to an assembly for insertion of an analyte sensor. The assembly can include an applicator and a sensor control device configured to be worn on the skin of a user. The applicator can include an applicator housing, a sharp, a carrier that is configured to releasably retain the sensor control device, an applicator cap that is removably coupled with the applicator housing. In some embodiments, the applicator can further include a desiccant that is disposed in the applicator cap. The sensor control device can include a sensor control device housing, with sensor electronics disposed in the sensor control device housing. The sensor control device can also include the analyte sensor, which can include a proximal portion coupled with the sensor electronics, and a distal portion configured to be transcutaneously positioned through the skin of the user. The sensor control device further includes an adhesive patch comprising a distally-facing surface configured to adhere to the skin of the user, and an adhesive liner.

In many embodiments, the adhesive liner can include a proximal part that is removably coupled with the adhesive patch and a distal part that is coupled with the applicator cap. In some embodiments, the desiccant can be configured to secure the distal part of the adhesive liner in the applicator cap, and the proximal part of the adhesive liner can be configured such that removal of the applicator cap causes removal of the proximal part of the adhesive liner from the adhesive patch. In other embodiments, the distal part of the adhesive liner can include an engagement element, which can be configured to mate with a retention element of the applicator cap, and the adhesive liner can be configured such that removal of the applicator cap causes removal of the proximal part of the adhesive liner from the adhesive patch.

In many embodiments, the sensor control device can further include a sensor cap. The sensor cap can be configured to cover the analyte sensor and the sharp, wherein the sensor cap is removably coupled with the sensor control device housing, and wherein the sensor cap is further configured such that the removal of the applicator cap further causes removal of the sensor cap from the sensor control device housing. In some embodiments, the adhesive liner can include a proximal part that is removably coupled with the adhesive patch and a distal part that is coupled with the sensor cap. The distal part of the adhesive liner can comprise an aperture to receive the sensor cap, and the adhesive liner can be configured such that removal of the sensor cap and the applicator cap causes removal of the proximal part of the adhesive liner from the adhesive patch. In other embodiments, the sensor cap can include a retention element. The adhesive liner can be removably coupled with the adhesive patch, and the adhesive liner can further include an engagement element. The engagement element of the adhesive liner can be configured to mate with the retention element of the sensor cap, and the adhesive liner can be configured such that removal of the sensor cap and the applicator cap causes removal of the adhesive liner from the adhesive patch. According to an aspect of the embodiments, the adhesive liner can further include a plurality of liner tabs. A first liner tab of the plurality of liner tabs can be coupled with the sensor cap at a first position or on a first side, and a second liner tab of the plurality of liner tabs can be coupled with the sensor cap at a second position or on a second side, and the adhesive liner can be configured such that removal of the sensor cap and the applicator cap causes removal of the adhesive liner from the adhesive patch. For example, the first side or position may be substantially opposite the second side or position. According to another aspect of the embodiments, the sensor cap can include a ratchet feature. The adhesive liner can be removably coupled with the adhesive patch, and the adhesive liner can further include a spiral pattern and a liner tab. The liner tab of the adhesive liner can be configured to mate with the ratchet feature of the sensor cap, and the adhesive liner can be configured such that removal of the sensor cap and the applicator cap causes removal of the adhesive liner from the adhesive patch.

According to another aspect of the embodiments, the sensor control device can further include a collar that is configured to snap-fit into the sensor cap. The adhesive liner can be removably coupled with the adhesive patch, and the adhesive liner can further include an outer portion and a center portion, wherein the center portion of the adhesive patch comprises a plurality of segments. Each of the plurality of segments of the center portion of the adhesive liner, or a plurality of the segments, can be configured to mate with the collar individually, and the adhesive liner can be configured such that removal of the sensor cap and the applicator cap causes removal of the adhesive liner from the adhesive patch.

In some embodiments, an assembly for insertion of an analyte sensor is provided. The assembly can include an applicator having an applicator housing, a sharp, a carrier configured to releasably retain a sensor control device, and an applicator cap removably coupled with the applicator housing. The assembly can further include a sensor control device configured to be worn on skin of a user, which includes a sensor control device housing, sensor electronics disposed in the sensor control device housing, the analyte sensor, a sensor cap, a collar configured to snap-fit into the sensor cap, an adhesive patch comprising a distally-facing surface configured to adhere to the skin of the user, and an adhesive liner. The adhesive liner can be configured such that removal of the applicator cap and or the sensor cap causes removal of the adhesive liner from the adhesive patch.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of a sensor applicator, reader device, monitoring system, network, and remote system.

FIG. 2A is a block diagram depicting an example embodiment of a reader device.

FIGS. 2B and 2C are block diagrams depicting example embodiments of sensor control devices.

FIG. 3A is a proximal perspective view depicting an example embodiment of a user preparing a tray for an assembly.

FIG. 3B is a side view depicting an example embodiment of a user preparing an applicator for an assembly.

FIG. 3C is a proximal perspective view depicting an example embodiment of a user inserting an applicator into a tray during an assembly.

FIG. 3D is a proximal perspective view depicting an example embodiment of a user removing an applicator from a tray during an assembly.

FIG. 3E is a proximal perspective view depicting an example embodiment of a user applying a sensor using an applicator.

FIG. 3F is a proximal perspective view depicting an example embodiment of a user with an applied sensor and a used applicator.

FIG. 4A is a side view depicting an example embodiment of an applicator coupled with a cap.

FIG. 4B is a side perspective view depicting an example embodiment of an applicator and cap decoupled.

FIG. 4C is a perspective view depicting an example embodiment of a distal end of an applicator and electronics housing.

FIG. 4D is a top perspective view of an example embodiment of an applicator.

FIG. 4E is a bottom perspective view of the applicator of FIG. 4D.

FIG. 4F is an exploded view of the applicator of FIG. 4D.

FIG. 4G is a side cutaway view of the applicator of FIG. 4D.

FIG. 5 is a proximal perspective view depicting an example embodiment of a tray with sterilization lid coupled.

FIG. 6A is a proximal perspective cutaway view depicting an example embodiment of a tray with sensor delivery components.

FIG. 6B is a proximal perspective view depicting sensor delivery components.

FIG. 7A is a side view depicting an example embodiment of an applicator housing.

FIG. 7B is a perspective view depicting an example embodiment of a distal end of an applicator housing.

FIG. 7C is a side cross-sectional view depicting an example embodiment of an applicator housing.

FIGS. 7D and 7E are side cross-sectional views depicting a locking rib portion of an example embodiment of an applicator housing with a portion of a sheath.

FIG. 7F is a side view of an example embodiment of an applicator housing.

FIG. 7G is a bottom perspective view of the applicator housing of FIG. 7F.

FIG. 7H is a side cutaway view of the applicator housing of FIG. 7F.

FIG. 7I is bottom perspective view of a cap.

FIG. 7J is a side cutaway view of the cap of FIG. 7I.

FIG. 7K is a top view of the cap of FIG. 7I.

FIG. 7L is a cutaway view of a desiccant in a cap.

FIG. 7M-7N are bottom and top perspective views of an example embodiment of a desiccant in accordance with the disclosed subject matter.

FIGS. 7O-7P are enlarged cross-sectional side views of the interface between the housing and cap.

FIGS. 7Q-7R are enlarged cross-sectional side views of the housing and cap, respectively.

FIGS. 7S-7T are side cutaway views of the cap of FIG. 7I.

FIG. 7U is a top perspective view of an example embodiment of an elastomeric plug, according to one or more embodiments.

FIG. 7V is a partial cross-sectional perspective view of an example embodiment of a sensor cap and an elastomeric plug.

FIG. 8A is a side view depicting an example embodiment of a sheath.

FIG. 8B is a perspective view depicting an example embodiment of a proximal end of a sheath.

FIG. 8C is a close-up perspective view depicting an example embodiment of a distal side of a detent snap of a sheath.

FIG. 8D is a side view depicting an example embodiment of features of a sheath.

FIG. 8E is an end view of an example embodiment of a proximal end of a sheath.

FIGS. 8F to 8H are perspective views depicting another example embodiment of a sheath in various stages of assembly with other applicator components.

FIG. 8I is a side view of a sheath in accordance with the disclosed subject matter.

FIG. 8J is a close-up view of a detent snap of the sheath of FIG. 8I.

FIG. 8K is a top view of the sheath of FIG. 8I.

FIG. 8L is a perspective view of the sheath of FIG. 8I.

FIG. 8M is a side cutaway view of the sheath of FIG. 8I.

FIG. 8N is a close-up view a lock arm of the sheath of FIG. 8I and the lock arm's engagement with a cap and a sensor carrier.

FIG. 8O is a close-up view of a rib of the sheath of FIG. 8I and the rib's engagement with a sensor carrier.

FIG. 9A is a proximal perspective view depicting an example embodiment of a sensor carrier.

FIG. 9B is a distal perspective view depicting an example embodiment of a sensor carrier.

FIG. 9C is a distal perspective view depicting another example embodiment of a sensor carrier.

FIG. 9D is a top perspective view of a sensor carrier.

FIG. 9E is a bottom view of the sensor carrier of FIG. 9D.

FIG. 10A is a perspective view of a sharp carrier.

FIG. 10B is a side cutaway view of the sharp carrier of FIG. 10A.

FIG. 10C is a perspective view of a sharp carrier.

FIG. 10D is a side cutaway view of the sharp carrier of FIG. 10C.

FIGS. 11A and 11B are top and bottom perspective views, respectively, depicting an example embodiment of a sensor module.

FIGS. 12A and 12B are perspective and compressed views, respectively, depicting an example embodiment of a sensor connector.

FIG. 13 is a perspective view depicting an example embodiment of a sensor.

FIG. 14 is a side view of another example embodiment of a sensor.

FIGS. 15A and 15B are isometric and partially exploded isometric views of an example embodiment of a connector assembly.

FIG. 15C is an isometric bottom view of the connector of FIGS. 15A-15B.

FIGS. 15D and 15E are isometric and partially exploded isometric views of another example embodiment of a connector assembly.

FIG. 15F is an isometric bottom view of the connector of FIGS. 15D-15E.

FIG. 16 is a perspective view depicting an example embodiment of a sharp module.

FIGS. 17A and 17B are isometric and side views, respectively, of another example embodiment of a sensor control device.

FIGS. 18A and 18B are exploded isometric top and bottom views, respectively of the sensor control device of FIGS. 17A-17B.

FIG. 18C is a top perspective view of a cell battery.

FIG. 19 is a cross-sectional side view of an assembled sealed subassembly.

FIGS. 20A-20C are progressive cross-sectional side views showing assembly of the sensor applicator with the sensor control device of FIGS. 17A-17B.

FIGS. 21A and 21B are perspective and top views, respectively, of the cap post of FIG. 20C.

FIG. 22 is a cross-sectional side view of the sensor control device of FIGS. 17A-17B.

FIGS. 23A and 23B are cross-sectional side views of the sensor applicator ready to deploy the sensor control device to a target monitoring location.

FIG. 24 is an isometric bottom view of the applicator housing.

FIG. 25 is an isometric bottom view of the housing with the sheath and other components at least partially positioned therein.

FIG. 26 is an enlarged cross-sectional side view of the sensor applicator with the sensor control device installed therein.

FIG. 27A is an isometric top view of the cap.

FIG. 27B is an enlarged cross-sectional view of the engagement between the cap and the applicator housing.

FIGS. 28A and 28B are isometric views of the sensor cap and the collar, respectively.

FIGS. 29A and 29B are side and isometric views, respectively, of an example sensor control device, according to one or more embodiments of the present disclosure.

FIGS. 30A and 30B are exploded, isometric top and bottom views, respectively, of a sensor control device.

FIG. 30C is a top perspective view of the collar of FIG. 30A-30B.

FIGS. 30D and 30E are isometric top views of the sensor control device of FIG. 2 according to one or more embodiments.

FIG. 31 is a cross-sectional side view of the sensor control device of FIGS. 29A-29B and 30A-30B.

FIG. 32A is an exploded isometric view of a portion of another embodiment of the sensor control device of FIGS. 29A-29B and 30A-30B.

FIG. 32B is a cross-sectional view of the sharp hub and the mount of FIG. 32A.

FIG. 33A is an isometric bottom view of the mount of FIGS. 29A-29B and 30A-30B.

FIG. 33B is an isometric top view of the sensor cap of FIGS. 29A-29B and 30A-30B.

FIGS. 34A and 34B are side and cross-sectional side views, respectively, of an example applicator.

FIG. 35 is a cross-sectional side view of the sensor control device positioned within the applicator cap.

FIG. 36 is a cross-sectional view of a sensor control device showing example interaction between the sensor and the sharp.

FIG. 37A is a side cross-sectional view of a sharp hub, sharp, and sensor, with the sensor in an unbiased position.

FIG. 37B is a side cross-sectional view of a sharp hub, sharp, and sensor, with the sensor in a biased position.

FIG. 37C is a closeup of a portion of a sharp.

FIGS. 38A-38F illustrate cross-sectional views depicting an example embodiment of an applicator during a stage of deployment.

FIG. 39 is a top view of an example embodiment of an adhesive liner.

FIG. 40 is a cross-sectional view of an example embodiment of an applicator assembly including an example embodiment of an adhesive liner.

FIG. 41 is a cross-sectional view of an example embodiment of an applicator assembly including an example embodiment of an adhesive liner.

FIG. 42 is a side cross-sectional view of the adhesive liner in the embodiment of FIG. 41.

FIG. 43 is a cross-sectional view of an example embodiment of a sensor control device including an example embodiment of an adhesive liner.

FIG. 44 is a top view of an example embodiment of an adhesive liner of FIG. 43.

FIG. 45A is a bottom view of an example embodiment of an adhesive liner.

FIG. 45B is a top view of the adhesive liner of FIG. 45A.

FIGS. 46A-46B are perspective views of an example embodiment of a sensor cap coupled with the adhesive liner of FIGS. 45A-45B.

FIG. 47 is a perspective view of an example embodiment of a sensor cap.

FIG. 48 is a perspective view of an example embodiment of an adhesive liner.

FIG. 49 is a perspective view of the sensor cap in FIG. 47 coupled with the adhesive liner of FIG. 48.

FIG. 50 is a perspective view of an example embodiment of an adhesive liner.

FIG. 51 is a perspective view of a sensor cap coupled with the adhesive liner of FIG. 50.

FIG. 52 is a perspective view of the sensor cap and adhesive liner of FIG. 51 in an applicator cap.

FIG. 53 is a top view of an example embodiment of an adhesive liner.

FIG. 54 is a perspective view of a sensor cap coupled with the adhesive liner of FIG. 53.

FIG. 55 is an enlarged view of a portion of FIG. 54.

FIG. 56 is a perspective view of an example embodiment of an adhesive liner.

FIG. 57 is a top view of the example embodiment of the adhesive liner of FIG. 56.

FIG. 58 is a bottom view of the embodiment of FIG. 56.

FIG. 59 is a top view of an example embodiment of an adhesive liner.

FIG. 60A is a perspective view of a sensor cap coupled with the adhesive liner of FIG. 56.

FIG. 60B is a perspective view of the collar in FIG. 60A.

FIGS. 61-62 are enlarged views of the sensor cap of FIG. 60A.

FIG. 63 is a top view of the embodiment of FIG. 59.

FIGS. 64A-64B are diagrams depicting the kiss-cutting process of an adhesive liner.

FIG. 65A is a perspective view of an example embodiment of an applicator assembly including an example embodiment of sensor control device and carrier.

FIG. 65B is an enlarged view of a portion of the embodiment of FIG. 65A.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates, which may need to be independently confirmed.

Generally, embodiments of the present disclosure include systems, devices, and methods for the removal of adhesive liners from sensor control devices for use with analyte sensor insertion applicators in in vivo analyte monitoring systems. An applicator, along with the electronics housing of the sensor control device, can be provided to the user in a sterile package. According to some embodiments, a structure separate from the applicator, such as a container, can also be provided to the user as a sterile package with a sensor module and a sharp module contained therein. The user can couple the sensor module to the electronics housing and can couple the sharp to the applicator with an assembly process that involves the insertion of the applicator into the container in a specified manner. In other embodiments, the applicator, sensor control device, sensor module, and sharp module can be provided in a single package. The applicator can be used to position the sensor control device on a human body with a sensor in contact with the wearer's bodily fluid. Other improvements and advantages are provided as well. The various configurations of these devices are described in detail by way of the embodiments which are only examples.

Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiments of In Vivo Analyte Monitoring System

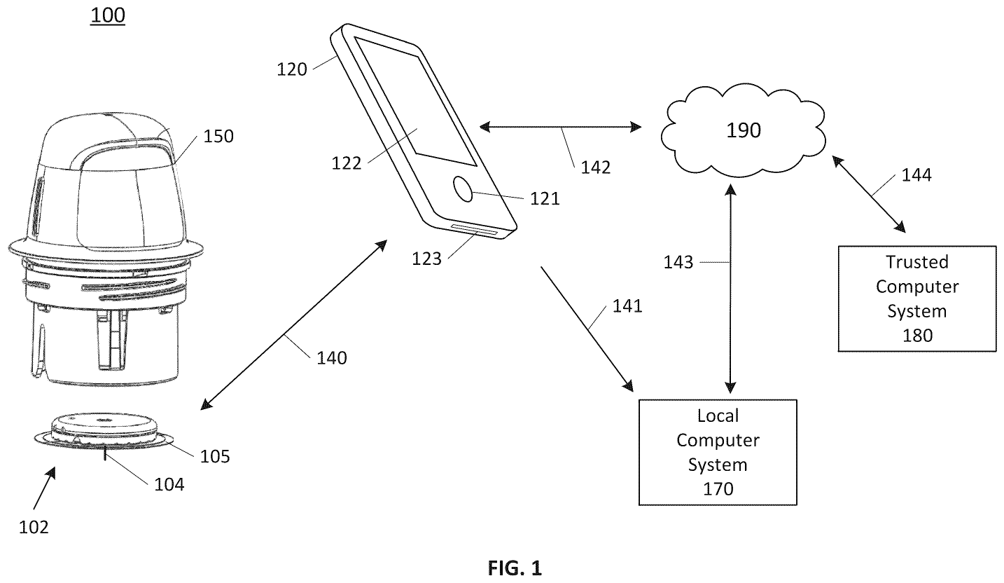

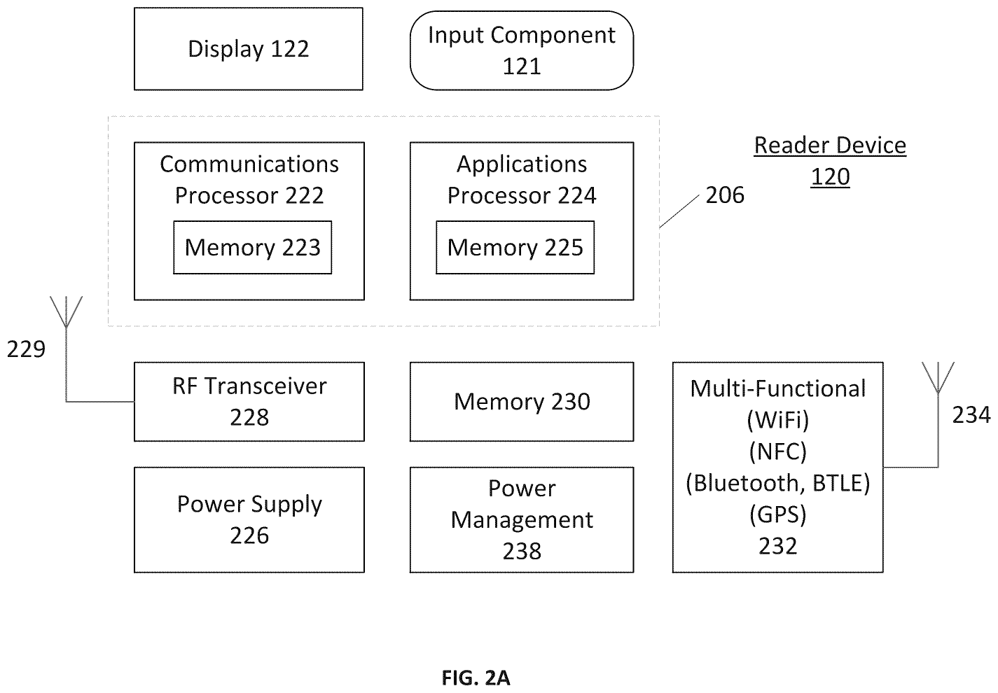

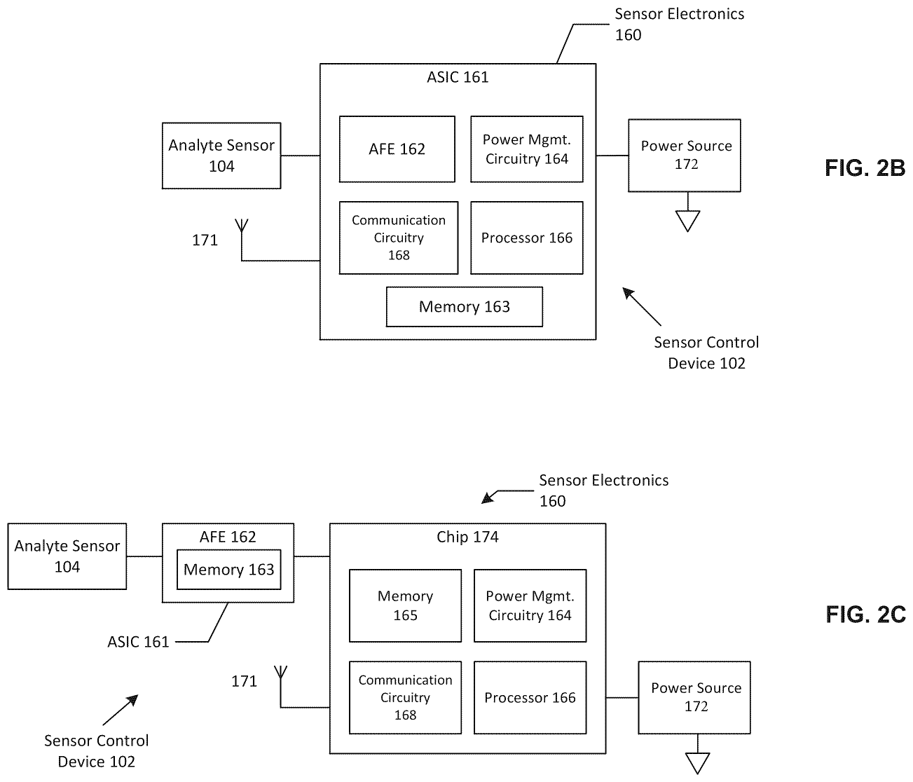

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can monitor applications installed in memory on reader device 120 using screen 122 and input 121 and the device battery can be recharged using power port 123. More detail about reader device 120 is set forth with respect to FIG. 2A below. Reader device 120 can communicate with local computer system 170 via a communication path 141 using a wired or wireless technique. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by wired or wireless technique as described previously. Network 190 can be any of a number of networks, such as private networks and public networks, local area or wide area networks, and so forth. A trusted computer system 180 can include a server and can provide authentication services and secured data storage and can communicate via communications path 144 with network 190 by wired or wireless technique.

### Example Embodiments of Reader Device

FIG. 2A is a block diagram depicting an example embodiment of a reader device configured as a smartphone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further included can be a multi-functional transceiver 232 which can communicate over Wi-Fi, NFC, Bluetooth, BTLE, and GPS with an antenna 234. As understood by one of skill in the art, these components are electrically and communicatively coupled in a manner to make a functional device.

### Example Embodiments of Sensor Control Devices

FIGS. 2B and 2C are block diagrams depicting example embodiments of sensor control device 102 having analyte sensor 104 and sensor electronics 160 (including analyte monitoring circuitry) that can have the majority of the processing capability for rendering end-result data suitable for display to the user. In FIG. 2B, a single semiconductor chip 161 is depicted, which can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161 or can be distributed amongst two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiments of Assembly Processes for Sensor Control Devices

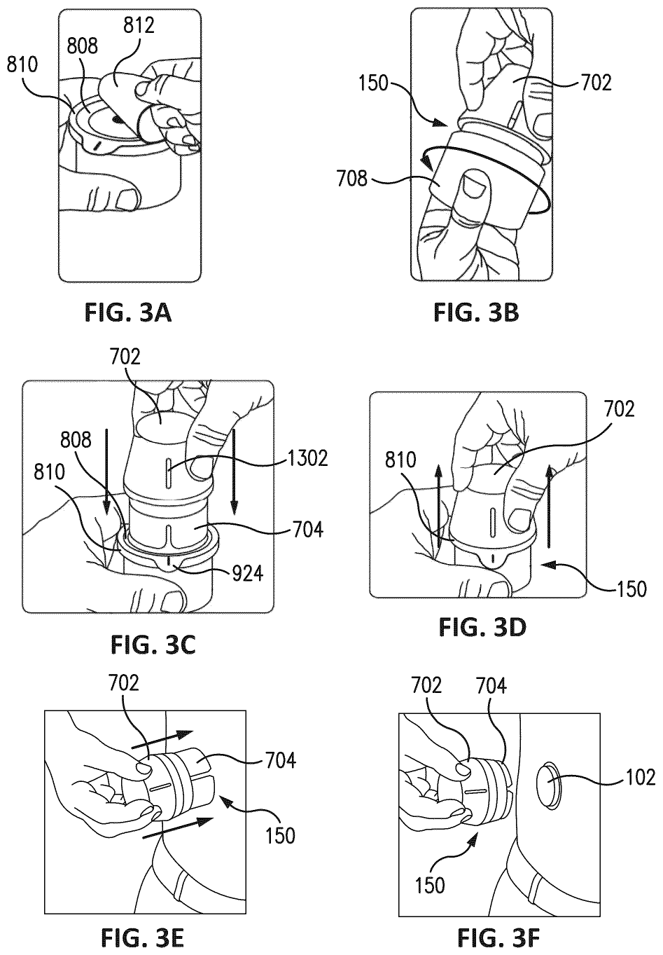

The components of sensor control device 102 can be acquired by a user in multiple packages requiring final assembly by the user before delivery to an appropriate user location. FIGS. 3A-3D depict an example embodiment of an assembly process for sensor control device 102 by a user, including preparation of separate components before coupling the components in order to ready the sensor for delivery. FIGS. 3E-3F depict an example embodiment of delivery of sensor control device 102 to an appropriate user location by selecting the appropriate delivery location and applying device 102 to the location.

FIG. 3A is a proximal perspective view depicting an example embodiment of a user preparing a container 810, configured here as a tray (although other packages can be used), for an assembly process. The user can accomplish this preparation by removing lid 812 from tray 810 to expose platform 808, for instance by peeling a non-adhered portion of lid 812 away from tray 810 such that adhered portions of lid 812 are removed. Removal of lid 812 can be appropriate in various embodiments so long as platform 808 is adequately exposed within tray 810. Lid 812 can then be placed aside.

FIG. 3B is a side view depicting an example embodiment of a user preparing an applicator 150 for assembly. Applicator 150 can be provided in a sterile package sealed by a cap 708. Preparation of applicator 150 can include uncoupling housing 702 from cap 708 to expose sheath 704 (FIG. 3C). This can be accomplished by unscrewing (or otherwise uncoupling) cap 708 from housing 702. Cap 708 can then be placed aside.

FIG. 3C is a proximal perspective view depicting an example embodiment of a user inserting an applicator 150 into a tray 810 during an assembly. Initially, the user can insert sheath 704 into platform 808 inside tray 810 after aligning housing orienting feature 1302 (or slot or recess) and tray orienting feature 924 (an abutment or detent). Inserting sheath 704 into platform 808 temporarily unlocks sheath 704 relative to housing 702 and also temporarily unlocks platform 808 relative to tray 810. At this stage, removal of applicator 150 from tray 810 will result in the same state prior to initial insertion of applicator 150 into tray 810 (i.e., the process can be reversed or aborted at this point and then repeated without consequence).

Sheath 704 can maintain position within platform 808 with respect to housing 702 while housing 702 is distally advanced, coupling with platform 808 to distally advance platform 808 with respect to tray 810. This step unlocks and collapses platform 808 within tray 810. Sheath 704 can contact and disengage locking features (not shown) within tray 810 that unlock sheath 704 with respect to housing 702 and prevent sheath 704 from moving (relatively) while housing 702 continues to distally advance platform 808. At the end of advancement of housing 702 and platform 808, sheath 704 is permanently unlocked relative to housing 702. A sharp and sensor (not shown) within tray 810 can be coupled with an electronics housing (not shown) within housing 702 at the end of the distal advancement of housing 702. The operation and interaction of the applicator 150 and tray 810 are further described below.

FIG. 3D is a proximal perspective view depicting an example embodiment of a user removing an applicator 150 from a tray 810 during an assembly. A user can remove applicator 150 from tray 810 by proximally advancing housing 702 with respect to tray 810 or other motions having the same end effect of uncoupling applicator 150 and tray 810. The applicator 150 is removed with sensor control device 102 (not shown) fully assembled (sharp, sensor, electronics) therein and positioned for delivery.

FIG. 3E is a proximal perspective view depicting an example embodiment of a user applying sensor control device 102 using applicator 150 to a target area of skin, for instance, on an abdomen or other appropriate location. Advancing housing 702 distally collapses sheath 704 within housing 702 and applies the sensor to the target location such that an adhesive layer on the bottom side of sensor control device 102 adheres to the skin. The sharp is automatically retracted when housing 702 is fully advanced, while the sensor (not shown) is left in position to measure analyte levels.

FIG. 3F is a proximal perspective view depicting an example embodiment of a user with sensor control device 102 in an applied position. The user can then remove applicator 150 from the application site.

System 100, described with respect to FIGS. 3A-3F and elsewhere herein, can provide a reduced or eliminated chance of accidental breakage, permanent deformation, or incorrect assembly of applicator components compared to prior art systems. Since applicator housing 702 directly engages platform 808 while sheath 704 unlocks, rather than indirect engagement via sheath 704, relative angularity between sheath 704 and housing 702 will not result in breakage or permanent deformation of the arms or other components. The potential for relatively high forces (such as in conventional devices) during assembly will be reduced, which in turn reduces the chance of unsuccessful user assembly.

### Example Embodiments of Applicators

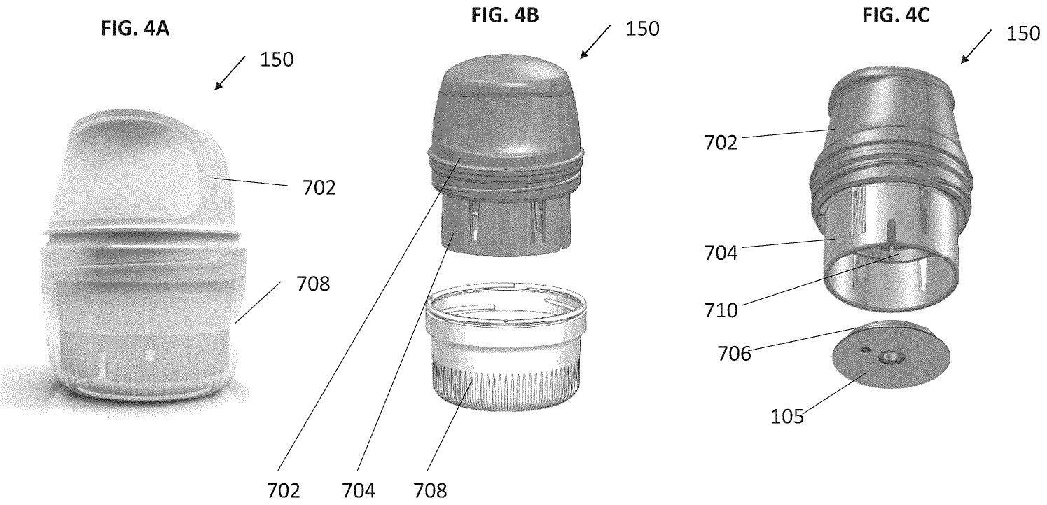

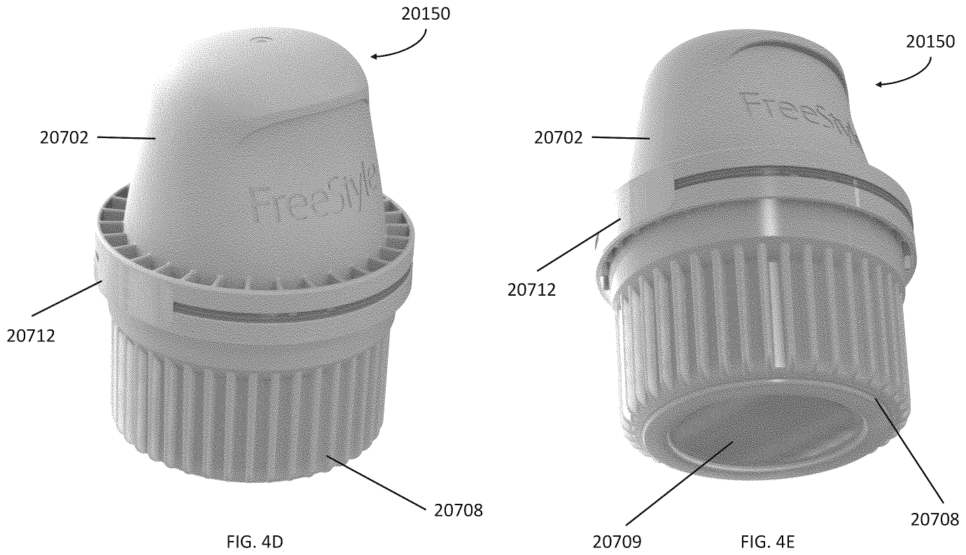

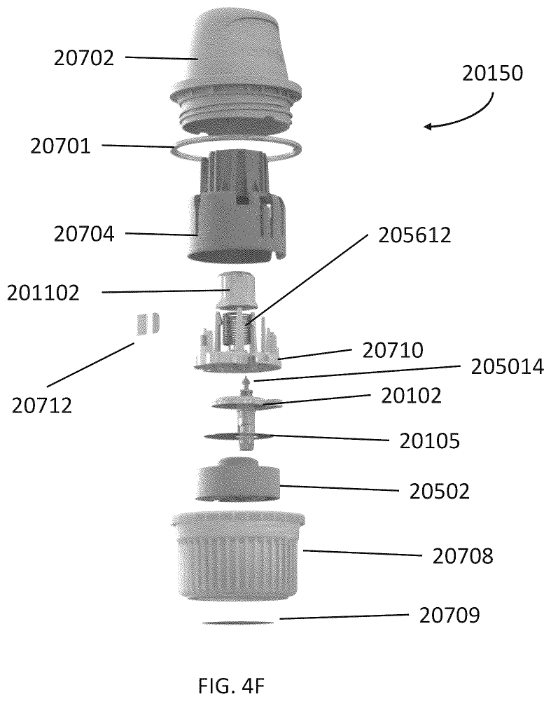

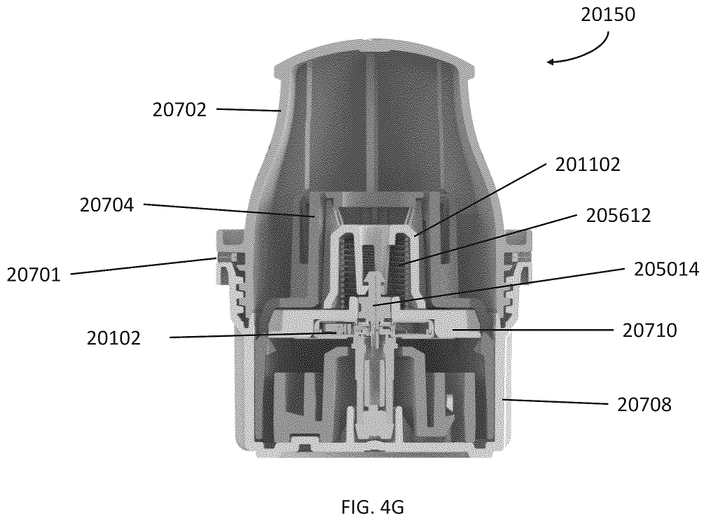

FIG. 4A is a side view depicting an example embodiment of an applicator 150 coupled with screw cap 708. This is an example of how applicator 150 is shipped to and received by a user, prior to assembly by the user with a sensor. FIG. 4B is a side perspective view depicting applicator 150 and cap 708 after being decoupled. FIG. 4C is a perspective view depicting an example embodiment of a distal end of an applicator 150 with electronics housing 706 and adhesive patch 105 removed from the position they would have retained within sensor carrier 710 of sheath 704, when cap 708 is in place.

Referring to FIG. 4D-G for purpose of illustration and not limitation, the applicator 20150 can be provided to a user as a single integrated assembly. FIGS. 4D and 4E provide perspective top and bottom views, respectively, of the applicator 20150, FIG. 4F provides an exploded view of the applicator device 20150 and FIG. 4G provides a side cut-away view. The perspective views illustrate how applicator 20150 is shipped to and received by a user. The exploded and cut-away views illustrate the components of the applicator 20150. The applicator 20150 can include a housing 20702, gasket 20701, sheath 20704, sharp carrier 201102, spring 205612, sensor carrier 20710 (also referred to as a “puck carrier”), sharp hub 205014, sensor control device (also referred to as a “puck”) 20102, adhesive patch 20105, desiccant 20502, cap 20708, serial label 20709, and tamper evidence feature 20712. As received by a user, only housing 20702, cap 20708, tamper evidence feature 20712, and label 20709 are visible. The tamper evidence feature 20712 can be, for example, a sticker coupled to each of the housing 20702 and the cap 20708, and tamper evidence feature 20712 can be damaged, for example, irreparably, by uncoupling housing 20702 and cap 20708, thereby indicating to a user that the housing 20702 and cap 20708 have been previously uncoupled. These features are described in greater detail below.

### Example Embodiments of Tray and Sensor Module Assembly

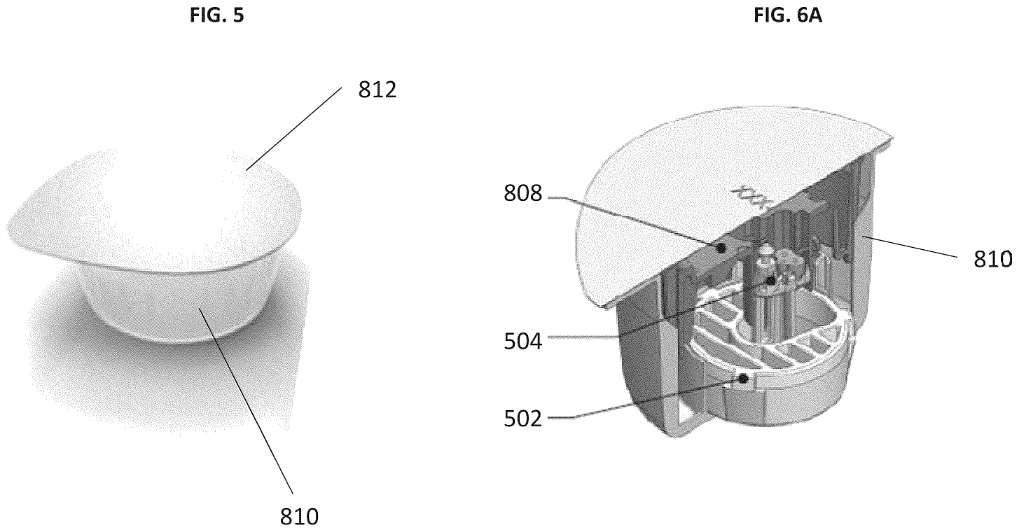

FIG. 5 is a proximal perspective view depicting an example embodiment of a tray 810 with sterilization lid 812 removably coupled thereto, which may be representative of how the package is shipped to and received by a user prior to assembly.

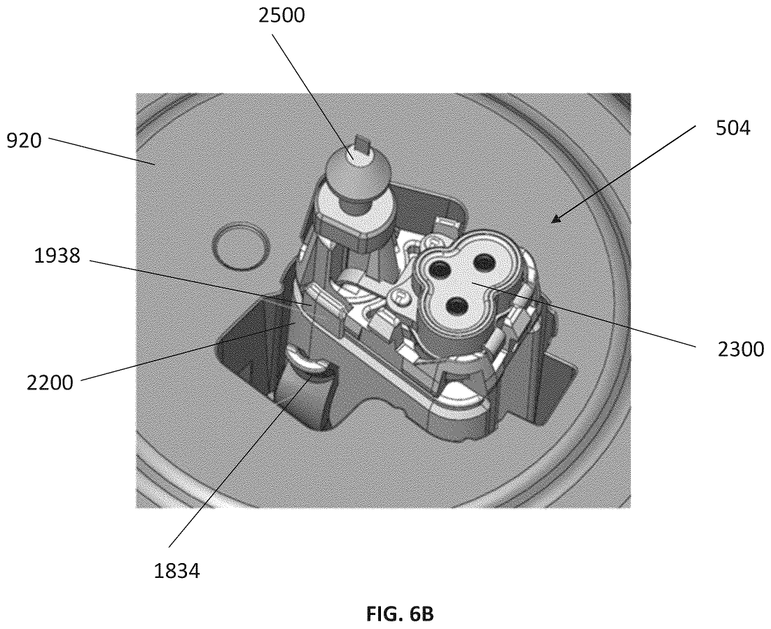

FIG. 6A is a proximal perspective cutaway view depicting sensor delivery components within tray 810. Platform 808 is slidably coupled within tray 810. Desiccant 502 is stationary with respect to tray 810. Sensor module 504 is mounted within tray 810.

FIG. 6B is a proximal perspective view depicting sensor module 504 in greater detail. Here, retention arm extensions 1834 of platform 808 releasably secure sensor module 504 in position. Module 2200 is coupled with connector 2300, sharp module 2500 and sensor (not shown) such that during assembly they can be removed together as sensor module 504.

### Example Embodiments of Applicator Housings and Caps

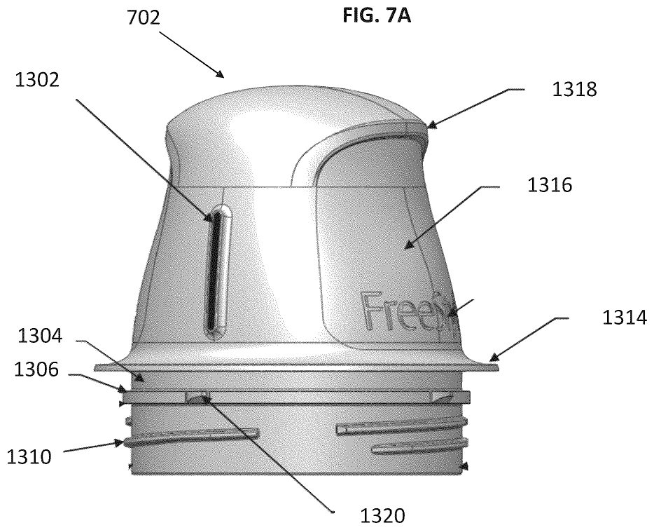

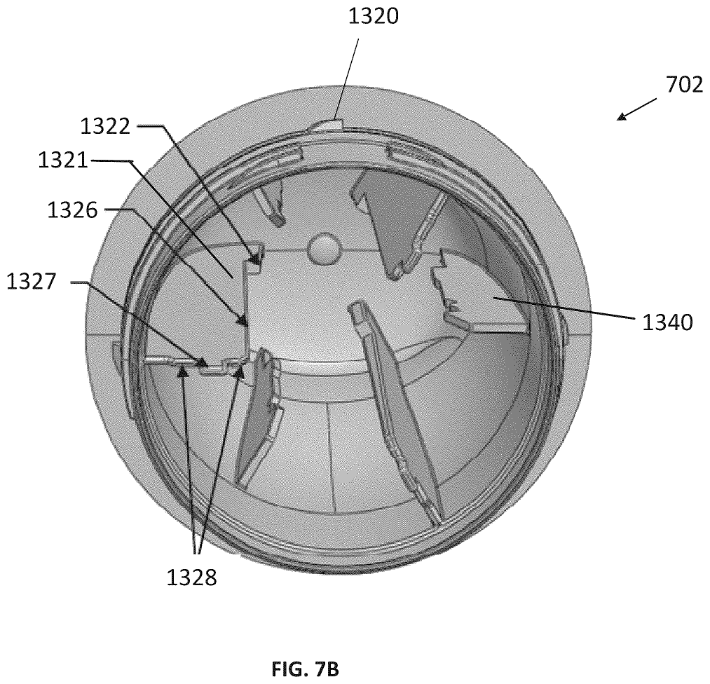

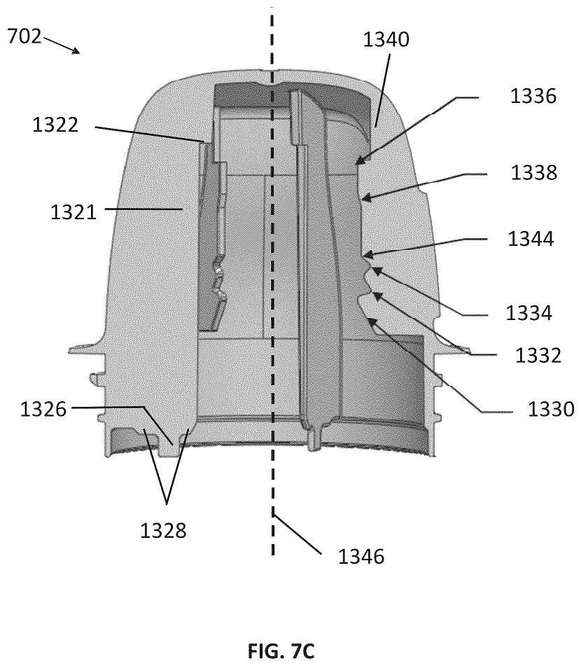

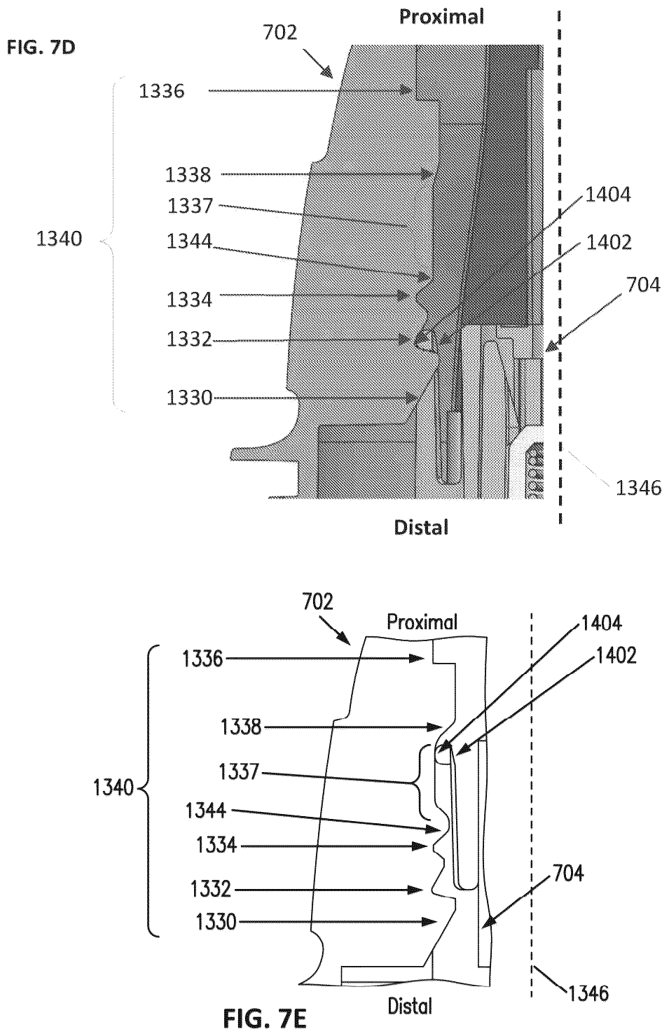

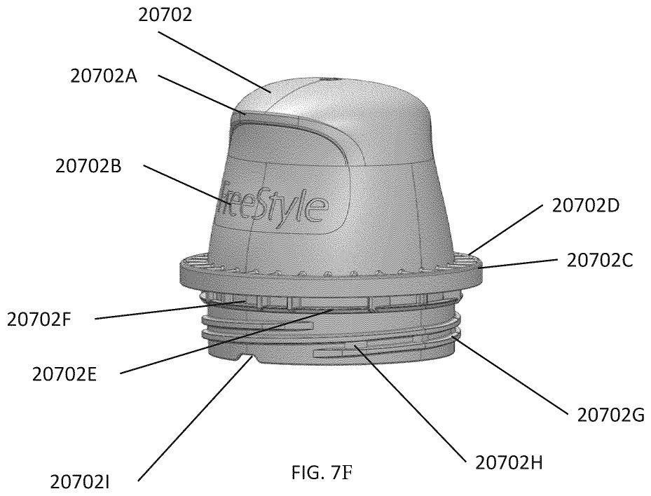

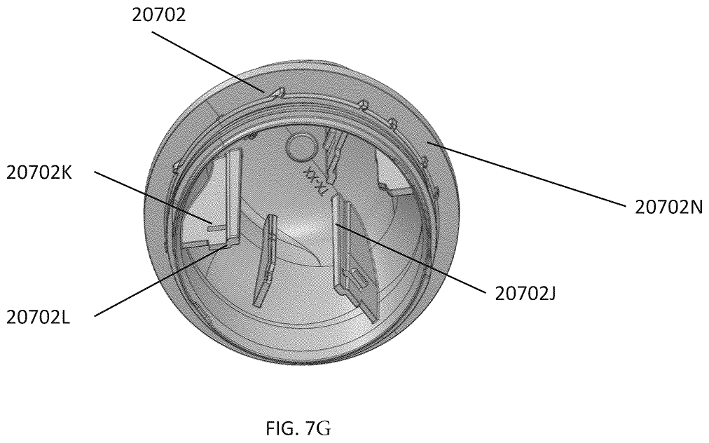

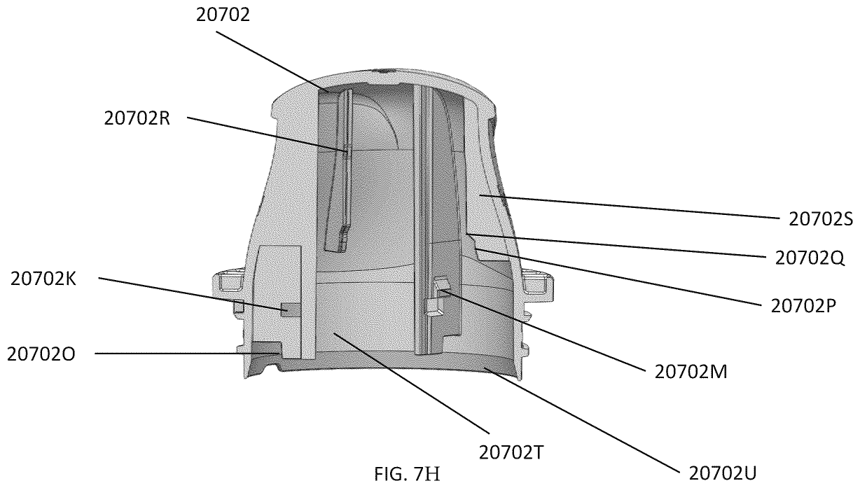

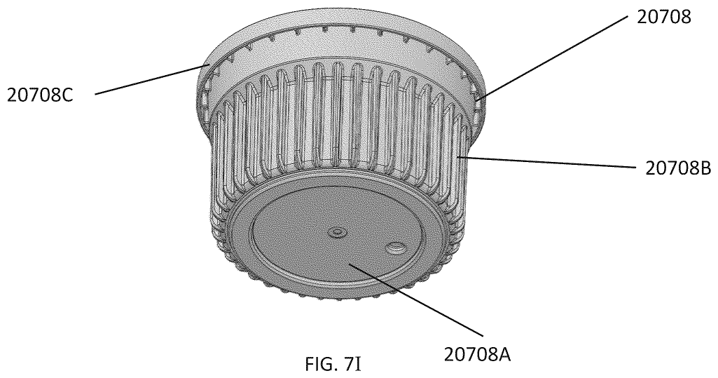

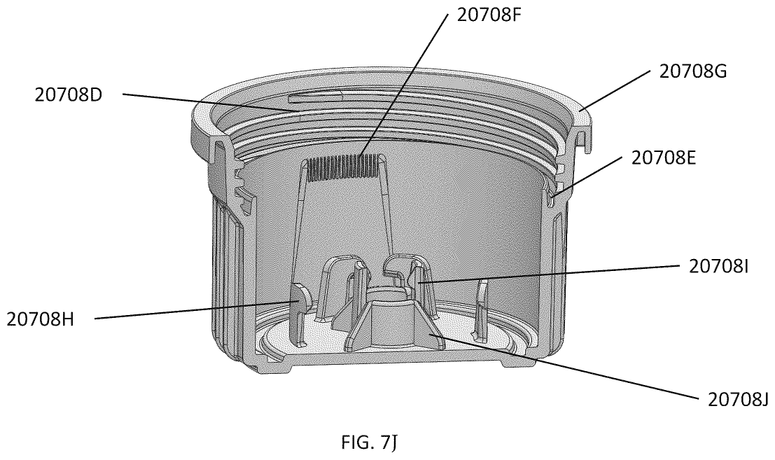

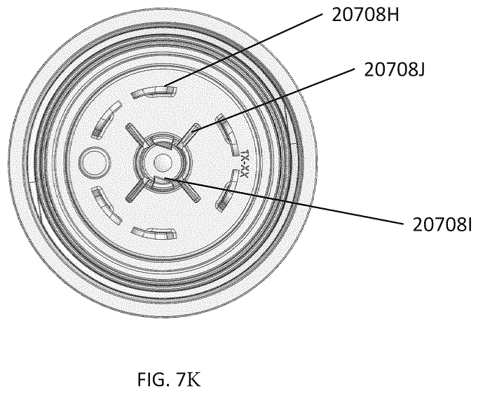

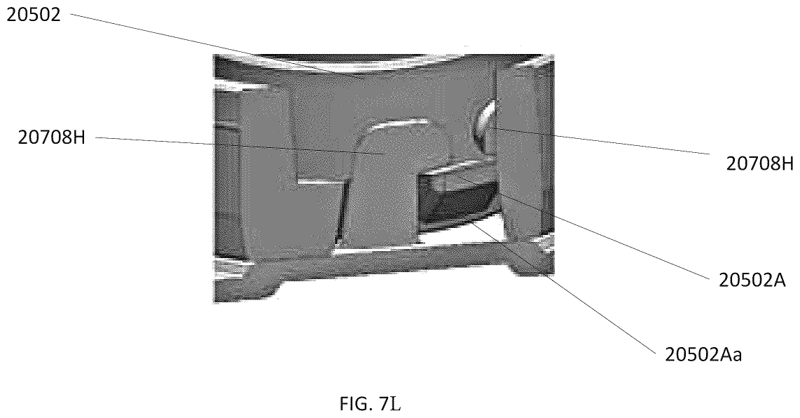

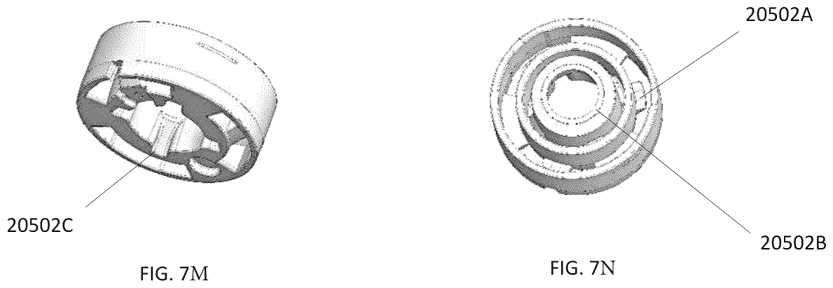

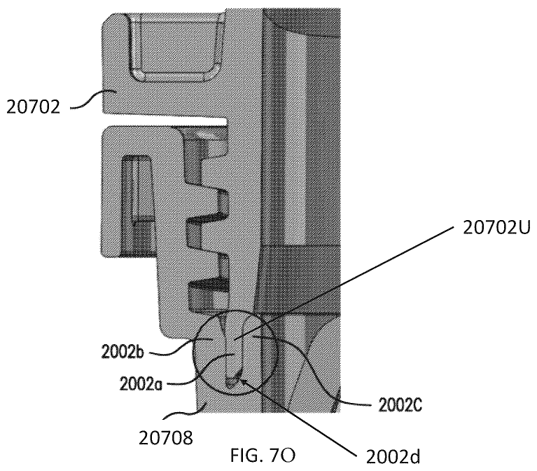

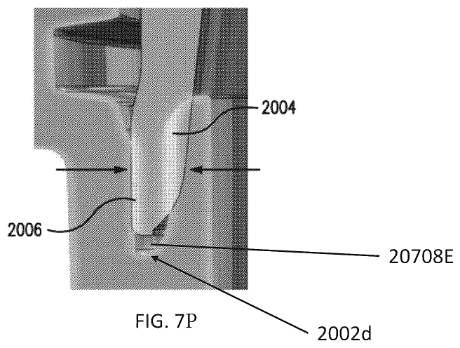

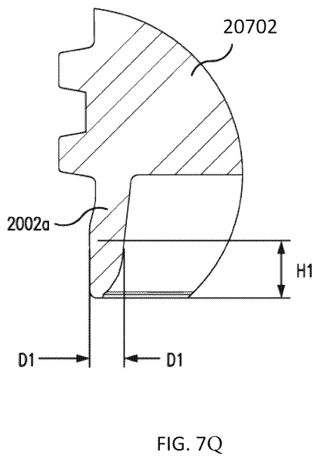

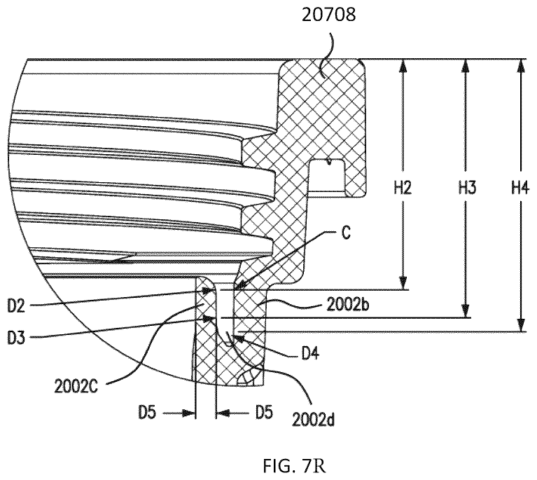

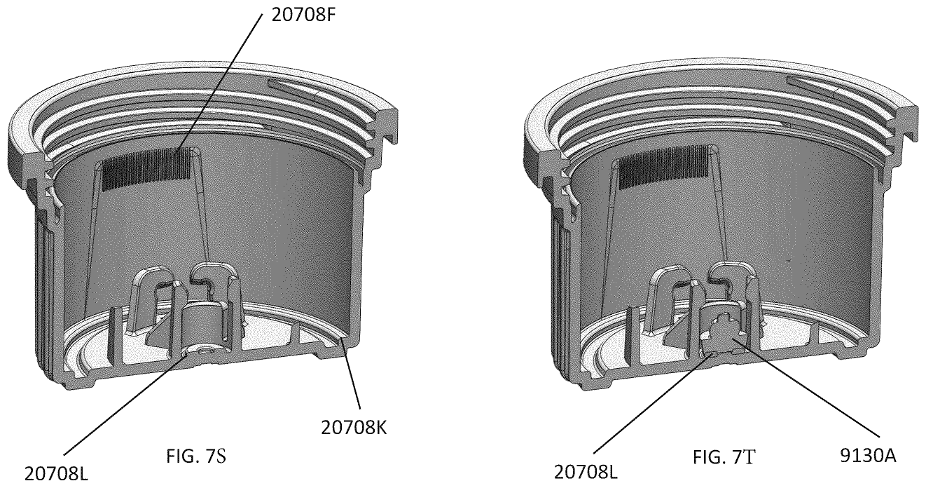

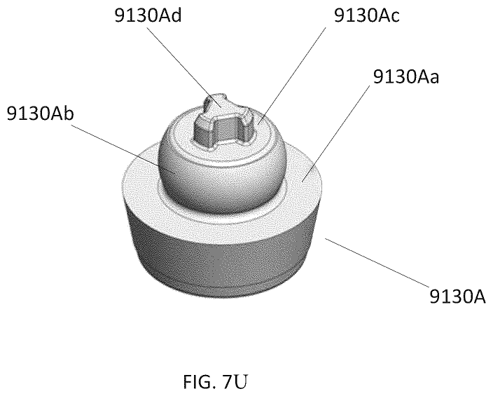

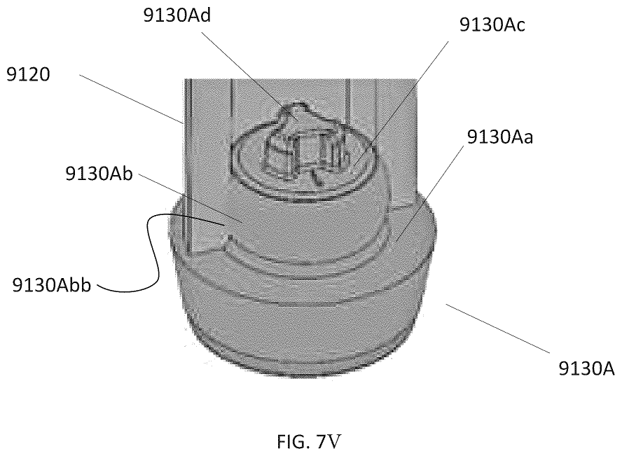

FIG. 7A is a side view depicting an example embodiment of the applicator housing 702 that can include an internal cavity with support structures for applicator function. A user can push housing 702 in a distal direction to activate the applicator assembly process and then also to cause delivery of sensor control device 102, after which the cavity of housing 702 can act as a receptacle for a sharp. In the example embodiment, various features are shown including housing orienting feature 1302 for orienting the device during assembly and use. Tamper ring groove 1304 can be a recess located around an outer circumference of housing 702, distal to a tamper ring protector 1314 and proximal to a tamper ring retainer 1306. Tamper ring groove 1304 can retain a tamper ring so users can identify whether the device has been tampered with or otherwise used. Housing threads 1310 can secure housing 702 to complimentary threads on cap 708 (FIGS. 4A and 4B) by aligning with complimentary cap threads and rotating in a clockwise or counterclockwise direction. A side grip zone 1316 of housing 702 can provide an exterior surface location where a user can grip housing 702 in order to use it. Grip overhang 1318 is a slightly raised ridge with respect to side grip zone 1316 which can aid in case of removal of housing 702 from cap 708. A shark tooth 1320 can be a raised section with a flat side located on a clockwise edge to shear off a tamper ring (not shown), and hold tamper ring in place after a user has unscrewed cap 708 and housing 702. In the example embodiment four shark teeth 1320 are used, although more or less can be used as desired.

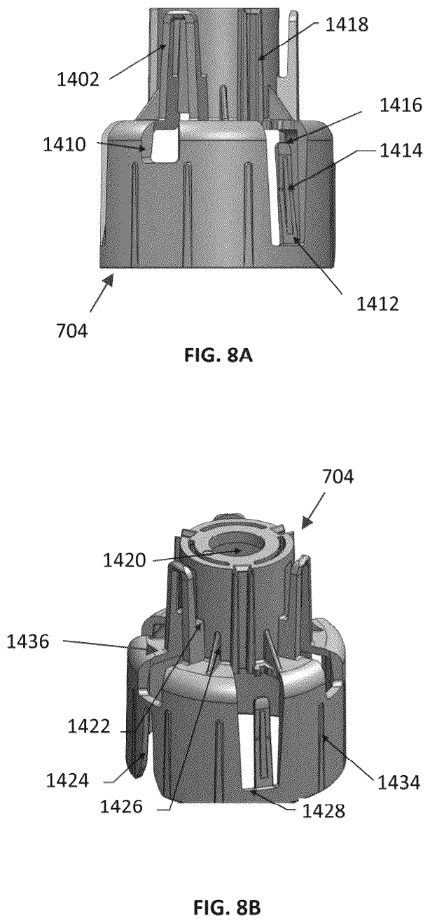

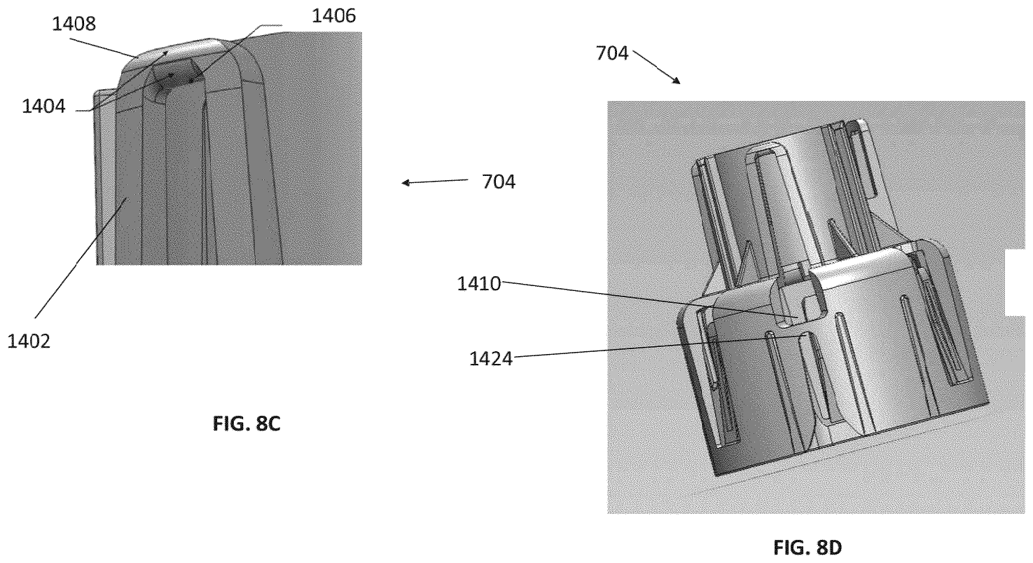

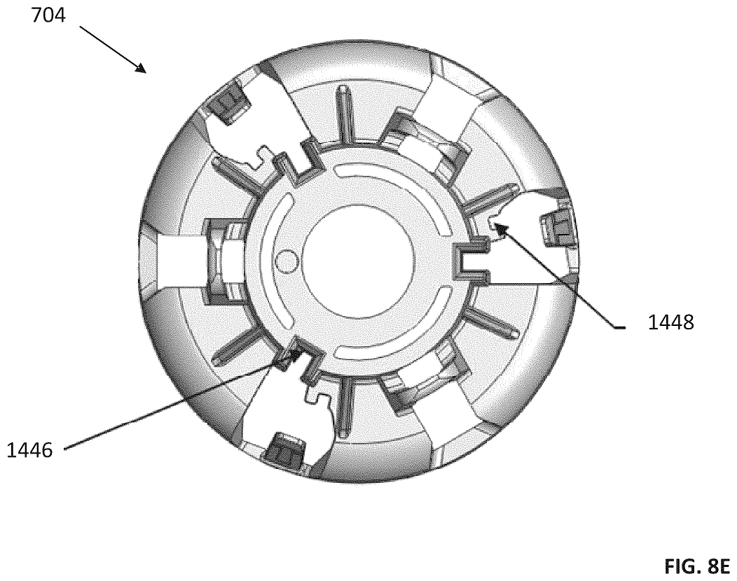

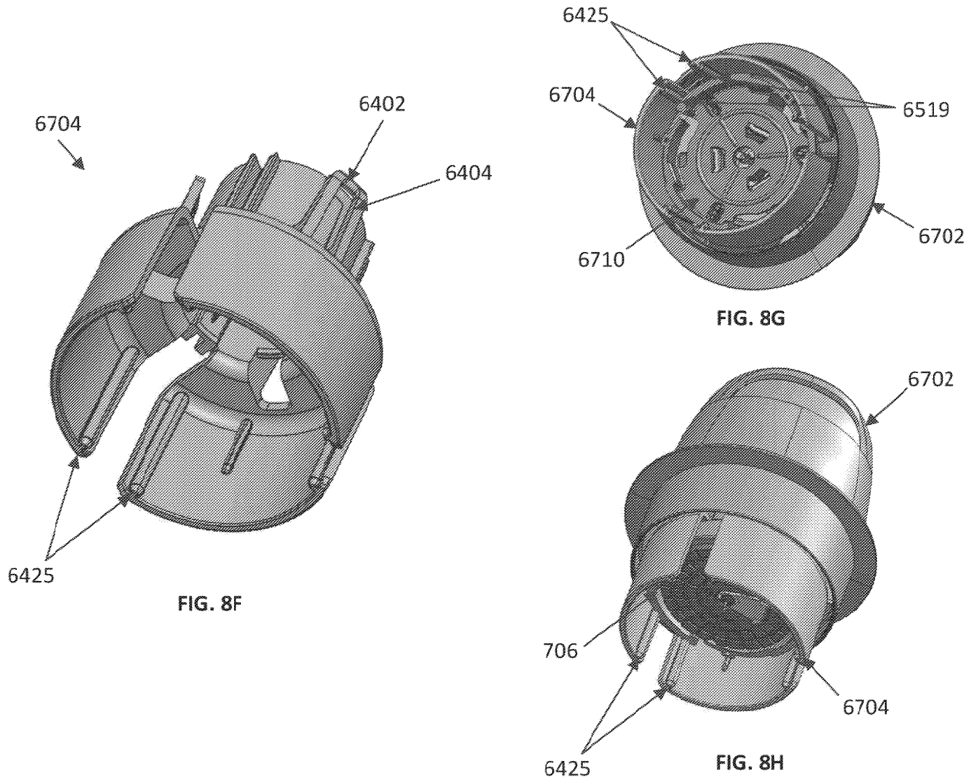

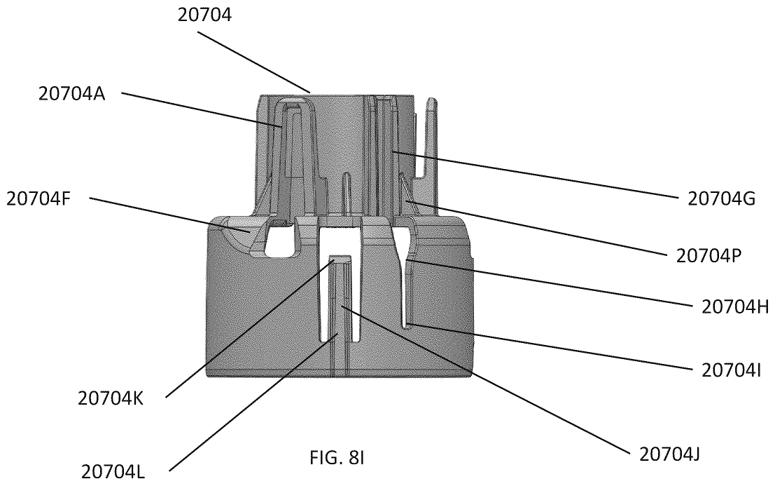

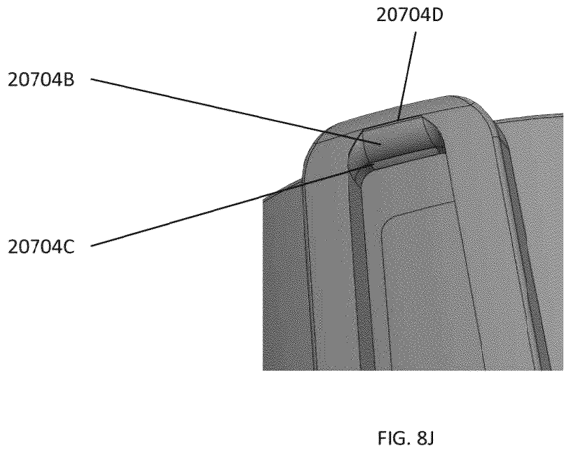

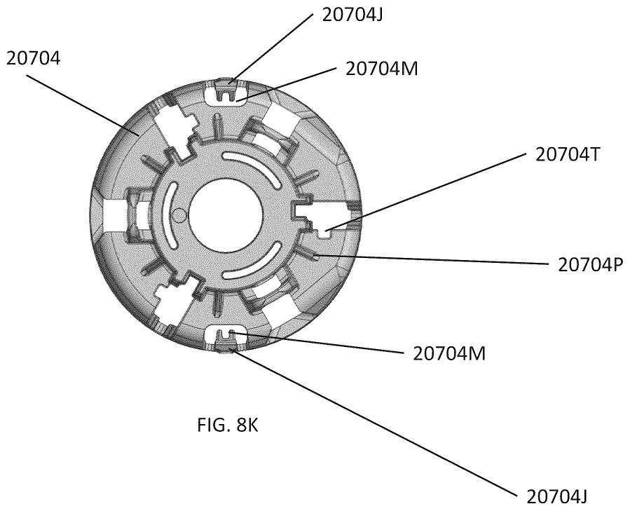

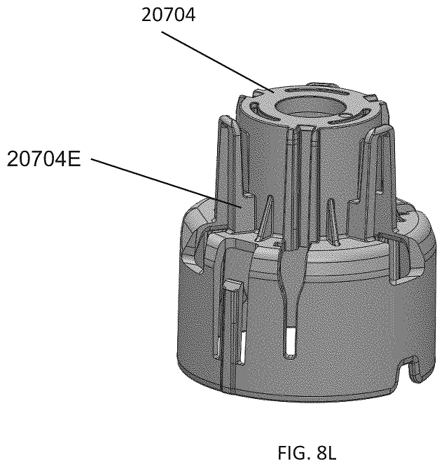

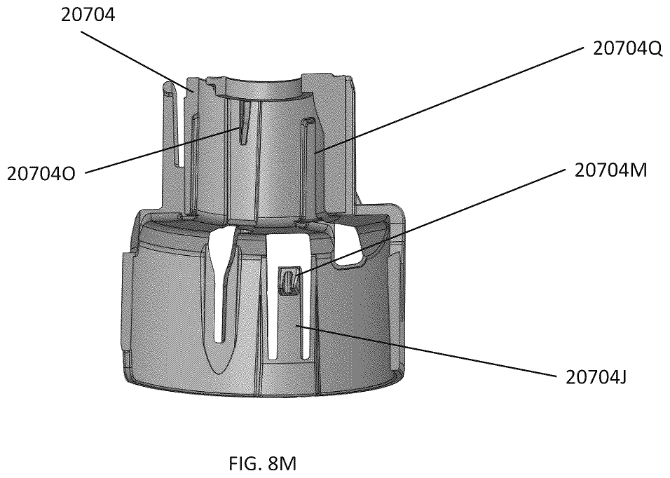

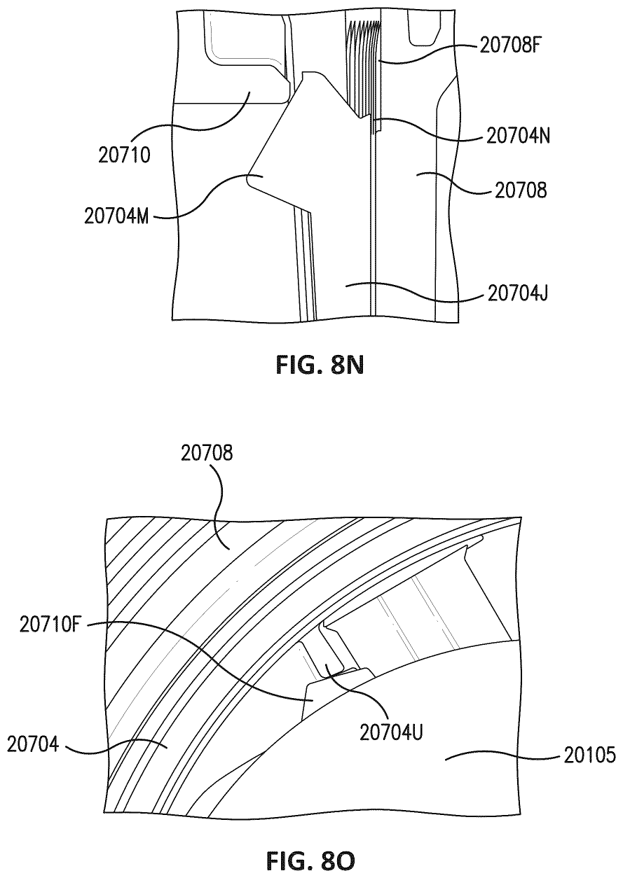

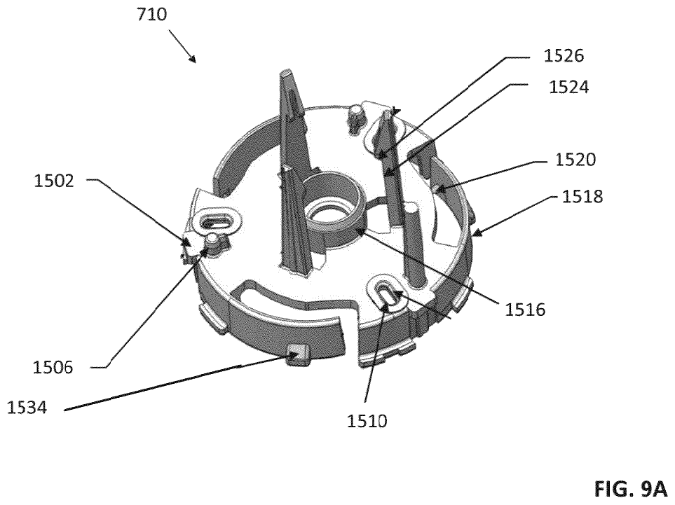

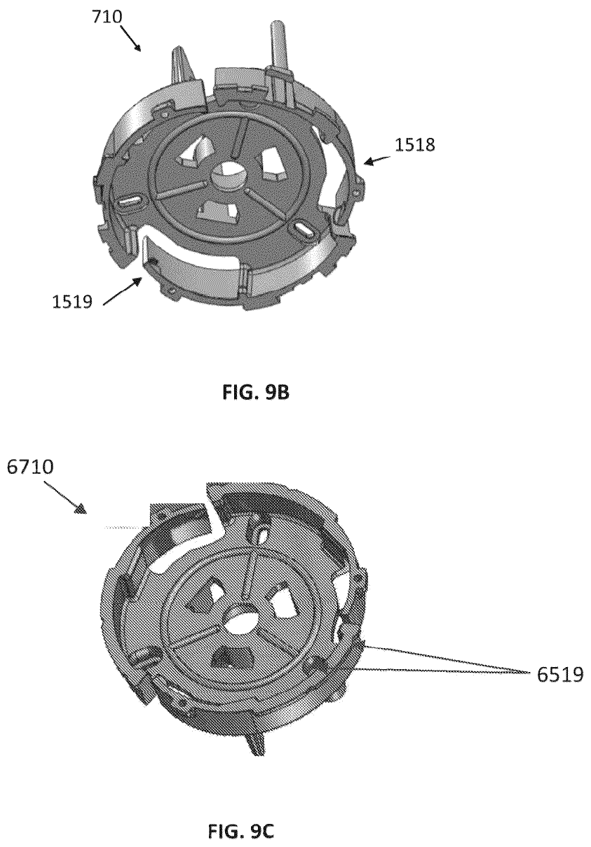

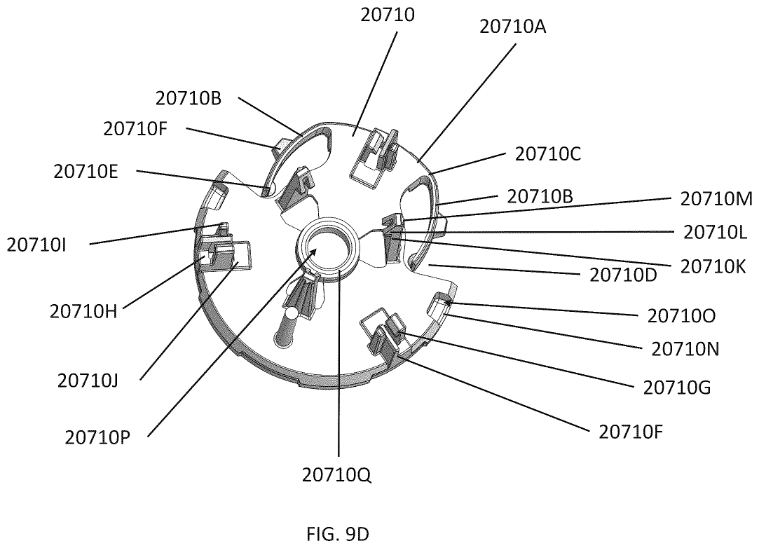

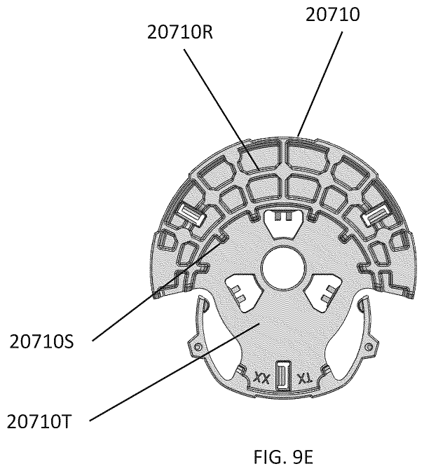

FIG. 7B is a perspective view depicting a distal end of housing 702. Here, three housing guide structures (or “guide ribs”) 1321 are located at 120-degree angles with respect to each other and at 60-degree angles with respect to locking structures (or “locking ribs”) 1340, of which there are also three at 120 degree angles with respect to each other. Other angular orientations, either symmetric or asymmetric, can be used, as well as any number of one or more structures 1321 and 1340. Here, each structure 1321 and 1340 is configured as a planar rib, although other shapes can be used. Each guide rib 1321 includes a guide edge (also called a “sheath guide rail”) 1326 that can pass along a surface of sheath 704 (e.g., guide rail 1418 described with respect to FIG. 8A). An insertion hard stop 1322 can be a flat, distally facing surface of housing guide rib 1321 located near a proximal end of housing guide rib 1321. Insertion hard stop 1322 provides a surface for a sensor carrier travel limiter face 1420 of a sheath 704 (FIG. 8B) to abut during use, preventing sensor carrier travel limiter face 1420 from moving any further in a proximal direction. A carrier interface post 1327 passes through an aperture 1510 (FIG. 9A) of sensor carrier 710 during an assembly. A sensor carrier interface 1328 can be a rounded, distally facing surface of housing guide ribs 1321 which interfaces with sensor carrier 710.

FIG. 7C is a side cross-section depicting an example embodiment of a housing. In the example embodiment, side cross-sectional profiles of housing guide rib 1321 and locking rib 1340 are shown. Locking rib 1340 includes sheath snap lead-in feature 1330 near a distal end of locking rib 1340 which flares outward from central axis 1346 of housing 702 distally. Each sheath snap lead-in feature 1330 causes detent snap round 1404 of detent snap 1402 of sheath 704 as shown in FIG. 8C to bend inward toward central axis 1346 as sheath 704 moves towards the proximal end of housing 702. Once past a distal point of sheath snap lead-in feature 1330, detent snap 1402 of sheath 704 is locked into place in locked groove 1332. As such, detent snap 1402 cannot be easily moved in a distal direction due to a surface with a near perpendicular plane to central axis 1346, shown as detent snap flat 1406 in FIG. 8C.

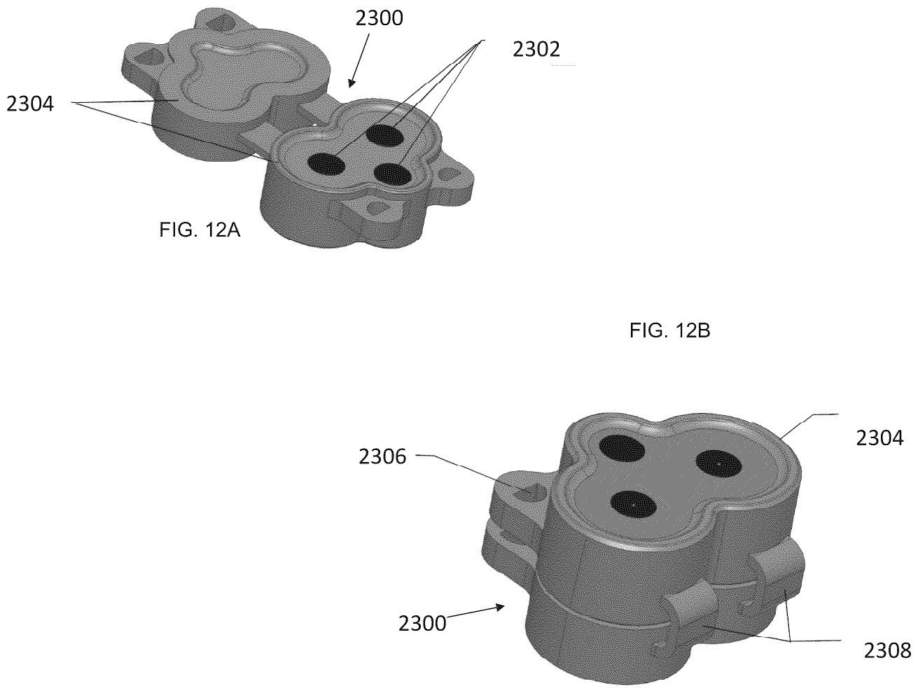

As housing 702 moves further in a distal direction toward the skin surface, and as sheath 704 advances toward the proximal end of housing 702, detent snaps 1402 shift into the unlocked grooves 1334, and applicator 150 is in an “armed” position, ready for use. When the user further applies force to the proximal end of housing 702, while sheath 704 is pressed against the skin, detent snap 1402 passes over firing detent 1344. This begins a firing sequence (as described, for example, with respect to FIGS. 12A-12D) due to release of stored energy in the deflected detent snaps 1402, which travel in a proximal direction relative to the skin surface, toward sheath stopping ramp 1338 which is slightly flared outward with respect to central axis 1346 and slows sheath 704 movement during the firing sequence. The next groove encountered by detent snap 1402 after unlocked groove 1334 is final lockout groove 1336 which detent snap 1402 enters at the end of the stroke or pushing sequence performed by the user. Final lockout recess 1336 can be a proximally-facing surface that is perpendicular to central axis 1346 which, after detent snap 1402 passes, engages a detent snap flat 1406 and prevents reuse of the device by securely holding sheath 704 in place with respect to housing 702. Insertion hard stop 1322 of housing guide rib 1321 prevents sheath 704 from advancing proximally with respect to housing 702 by engaging sensor carrier travel limiter face 1420.

FIGS. 7D and 7E are close-up side views of an example embodiment of locking rib 1340 of applicator housing 702, as detent snap 1402 of sheath 704 moves toward the proximal end of housing 702. FIG. 7D shows sheath 704 in a “locked” state, in which detent round 1404 of detent snap 1402 has already passed over sheath snap lead-in feature 1330 and is positioned in locked groove 1332 of locking rib 1340. As force is applied to the proximal end of housing 702, detent round 1404 is advanced proximally into unlocked groove 1334, placing applicator 150 into an “armed” position. When force is further applied to the proximal end of housing 702, applicator 150 is “fired,” as detent round 1404 is advanced proximally from the unlocked groove 1334 and passes over firing detent 1344. Thereafter, sheath 704 is further advanced proximally such that detent round 1404 is slidably advanced over firing surface 1337, as shown in FIG. 7E. In this embodiment, firing surface 1337 is substantially parallel to central axis 1346. As sheath 704 continues to advance proximally, detent round 1404 reaches sheath stopping ramp 1338 which slows the movement of sheath 704. Upon detent round 1404 reaching final lockout recess 1336, detent snap flat 1406 (not shown) is engaged and securely holds sheath 704 in place with respect to housing 702.

Referring to FIGS. 7F-7H, for purpose of illustration and not limitation, a housing 20702 in accordance with the disclosed subject matter is provided. Housing 20702 can be made of cyclic olefin copolymer, or other suitable materials, such as Polycarbonate or high density poly ethylene (HDPE). Housing 20702 can include one or more of the features described herein with regard to housings, wherein similar features can operate as described herein. For example, housing 20702 can include a grip overhang 20702A that can enable a user to securely grip housing 20702. Housing 20702 can have additional grip overhangs 20702A, for example, two grip overhands 20702A on opposite sides of the housing 20702. Housing 20702 can include a side grip zone 20702B disposed below the grip overhand 20702A. The side grip zone 20702B can be textured for improved gripping by a user. Housing 20702 can have additional side grip zones 20702B, for example, two side grip zones 20702B on opposite sides of housing 20702, each disposed below a grip overhang 20702A.

Housing 20702 can include a housing skirt 20702C, which can provide a surface for tamper evidence feature 20712. The housing skirt 20702C can be supported by a plurality of skirt stiffening ribs 20702D. The skirt stiffening ribs 20702D can provide support for the housing skirt 20702C and can help protect the applicator 20150 during a shock event, such as a drop. Additionally, the skirt stiffening ribs 20702D can be used to support the housing 20702 during manufacturing. The housing skirt 20702C and skirt stiffening ribs 20702D can provide stiffness against forces due to gasket compression and can help maintain gasket 20701 compression through shelf life. Housing 20702 can include a gasket retention ring 20702E and a plurality of gasket retention pockets 20702F, which can hold the gasket 20701 relative the housing 20702. For example, gasket retention ring 20702E can prevent lateral movement of the gasket 20701 and the gasket retention pockets 20702E can prevent rotation of the gasket 20701. The housing 20702 can include a plurality of gasket retention pockets, for example, 14 gasket retention pockets 20702E. Gasket sealing face 20702N that can seal against the gasket 20701. Housing 20702 can additionally or alternatively have an applicator cap sealing lip 20702U that can interface with the cap 20708, as described in greater detail below. Housing 20702 can have inner surface 20702T that can receive the sheath 20704.

Housing 20702 can include threads 20702G configure to engage with threads 20708D disposed on cap 20708. The threads can include radial limiting features 20702H, which can limit radial deformation of the cap 20702G during a shock event, such as a drop. Housing 20702 can include a plurality of radial limiting features 20702H, for example, 6 radial limiting features 20702H. The radial limiting features 20702H can be protrusions from the housing and can close a gap with the threads 20708D disposed on cap 20708. This can limit oval deformation of the cap 20702H during a shock event, such as a drop. Preventing oval deformation of cap 20702H can, in turn, ensure that lock arms 20704J of sheath 20704 stay locked between the cap 20702 and the sensor carrier 20710 to limit movement of the sheath 20704 prior to removing cap 20702H (as described in greater detail below). Housing 20702 can further include a clearance notch 20702I for clearance of the sheath arms during firing.

The interior of housing 20702 can include a plurality of sensor carrier attachment features for receiving, aligning, and limiting movement of the sensor carrier 20710. For example, housing 20703 can include sheath guide rails 20702J, which can help to align and guide sheath 20704 as the sheath 20704 moves relative the housing 20702. Housing 20702 can include sensor carrier attach slots 20702K, which can engage and hold the sensor carrier 20710, and sensor carrier hard stops 20702L, that can limit axial movement of the sensor carrier 20710 relative the housing 20702. Housing 20702 can include sensor carrier biasing feature 20702M that can remove slop between the sensor carrier 20710 and the housing 20702 after assembly and sensor carrier radial limiting feature 20702O that can keep the sensor carrier radially aligned relative the housing 20702. Flat horizontal faces between sensor carrier attach slots 20702K and sensor carrier radial limiting feature 20702O can be used to stop the sheath 20704 at the end of a stroke. Corresponding features on the sheath 20704 can interact with these faces. The sensor carrier biasing feature 20702M can further limit rotation of the sensor carrier 20710 relative the housing 20702. Housing 20702 can include one or more of each of the sheath guide rails 20702J, sensor carrier attach slots 20702K, sensor carrier hard stops 20702L, sensor carrier radial limiting feature 20702O, and sensor carrier biasing feature 20702M, for example, three of each.

The interior of housing 20702 can further include a plurality sheath ribs 20702S for engaging the sheath 20704 for insertion, as described herein. Housing 20702 can include one or more sheath ribs 20702S, for example, three. Each sheath rib 20702S can include a sheath snap lead in feature 20702P configured to initially lead in the detent snap 20704A of sheath 20704 into the correct location. Housing 20702 can include a firing detent 20702Q. After the detent snap 20704A of sheath 20704 passes the firing detent 20702Q, the firing sequence can be initiated, and the sheath 20704 can travel toward the sheath stopping ramp 20702R. Sheath stopping ramp 20702 can slow the sheath 20704 at the end of firing.

Referring to FIGS. 7I-7M and 7O-7T for purpose of illustration, an example embodiment of a cap 20708 is provided. Cap 20708 can include one or more of the features described herein with regard to caps, wherein similar features can operate as described herein. Cap 20708 can be made of high-density polyethylene (HDPE) or any other suitable materials, such as Polypropylene or low-density polyethylene (LDPE). Cap 20708 can include a label surface 20708A configured to receive label 20709. Cap 20708 can include ribs 20708B which can provide strength and provide an improved gripping surface for a user. The cap 20708 can include tamper label ring 20708C, which can receive the tamper evidence feature 20712. The cap 20708 can also include a gasket sealing surface 20708G, configured to engage gasket 20701.

Internally, cap 20708 can include threads 20708D, which can engage threads 20702G disposed on the housing 20702. Cap 20708 can also include seal interface 20708E which can be configured to receive the applicator cap sealing lip 20702U to create a seal between the housing 20702 and the cap 20708.

FIGS. 7O-7R show an enlarged cross-sectional side view of the interface between housing 20702 and cap 20708. As illustrated, applicator cap sealing lip 20702U of housing 20702 includes a first axial extension 2002a and seal interface 20708E of cap 20708 provides a cavity 2002d mateable with the first axial extension 2002a. In the illustrated embodiment, the diameter of cavity 2002d formed from second axial extension 2002b and third axial extension 2002c of the cap 20708 is sized to receive the diameter of first axial extension 2002a of housing 20702 within cavity 2002d. For example, as shown in FIG. 7U, axial extension 2002a can have thickness D1 at height H1, as measured from distal edge of axial extension 2002a. Similarly, second axial extension 2002c can have a thickness D5 at height H3, as measured from proximal edge of cap 20708; cavity 2002d can have a thickness D2, D3, and D4 at heights H2, H3, and H4, respectively, as measured from proximal edge of cap 20708. In certain embodiments, D1 can measure 1 mm with a tolerance of +/−0.03 mm, D2, D3, D4 can have any suitable dimensions, H1 can measure 1.66 mm with a tolerance of +/−0.1 mm, H2 can measure 8.25 mm with a tolerance of +/−0.1 mm, H3 can measure 9.25 mm with a tolerance of +/−0.1 mm, H4 can measure 9.75 mm with a tolerance of +/−0.1 mm. In other embodiments, however, the reverse can be employed, where the diameter of first axial extension 2002a can be sized to receive the diameter of the second axial extension 2002b, without departing from the scope of the disclosure.

In each embodiment, two radial seals 2004, 2006 can be defined or otherwise provided at the interface between first and second axial extensions 2002a, b and radial seals 2004 and 2006 can help prevent migration of fluids or contaminants across the interface in either axial direction. Moreover, the dual radial seals described herein can accommodate tolerance and thermal variations combined with stress relaxation via a redundant sealing strategy. In the illustrated embodiment, dual radial seals 2004, 2006 utilize a “wedge” effect for effective sealing between first axial extension 2002a and second axial extension 2002b.

Cap 20708 can include one or more sets of crush ribs 20708F (see FIG. 7J), for example, two sets of crush ribs 20708F. The crush ribs 20708F can be configured to engage the sharp edge 20704N of lock arm 20704J during a shock event, for example a drop, as described in greater detail below (see e.g., FIG. 8N).

In accordance with the disclosed subject matter, cap 20708 can include one or more desiccant retention clips 20708H to retain the desiccant 20502 in the cap 20708 and limit rotation of the desiccant 20502 (see e.g., FIG. 7L). Cap 20708 can include any number of desiccant retention clips 20708H. For example, cap 20708 can include one, two, three, four, five, six, or more desiccant retention clips 20708H. As can be seen in FIG. 7K, cap 20708 can include three pairs of two desiccant retention clips 20708H each. Each pair of desiccant retention clips 20708H may correspond to a desiccant retention snap 20502A (see e.g., FIG. 7N) on desiccant 20502 (see e.g., FIG. 7L). As can be seen in FIG. 7L, desiccant 20502 can include a plurality of desiccant retention snaps 20502A and each desiccant retention snap 20502A may snap down into a pair of desiccant retention clip 20708H. For example, desiccant retention snap 20502A can include angled surface 20502Aa to radially expand clips 20708H when the desiccant is pressed down onto the cap until desiccant retention snap 20502A locks in place within desiccant retention clips 20708H. Desiccant retention clips 20708H can hold desiccant 20502 within cap 20708 to limit distal and rotational movement of desiccant 20502. As can be seen in FIG. 7M, desiccant 20502 can include catch pockets (not shown) to angularly position desiccant 20502 relative to desiccant retention clips 20708H. In some embodiments, as can be seen in FIG. 7M desiccant 20502 can be cylindrical in shape.

In accordance with disclosed subject matter, cap 20708 can include a ratchet 20708I to engage the sensor cap and remove the sensor cap when the cap 20708 is removed from the housing 20702, as described in greater detail below. According to some embodiments, ratchet 20708I can be provided in a cap 20708 without the use or presence of the desiccant retention clips 20708H described earlier. In other embodiments, ratchet 20708I and desiccant retention clips 20708H can be provided together with cap 20708. Cap 20708 can include a plurality of ribs 20708J to provide strength. Correspondingly, as can be seen in FIGS. 4G and 7M-7N, internal bore 20502B of desiccant 20502 can be sized and dimensioned to allow sufficient clearance for sensor cap to pass through and engage with ratchet 20708I of cap 20708; desiccant 20502 can include a plurality of rib pockets 20502C to provide clearance for plurality of ribs 20708J of cap 20708.

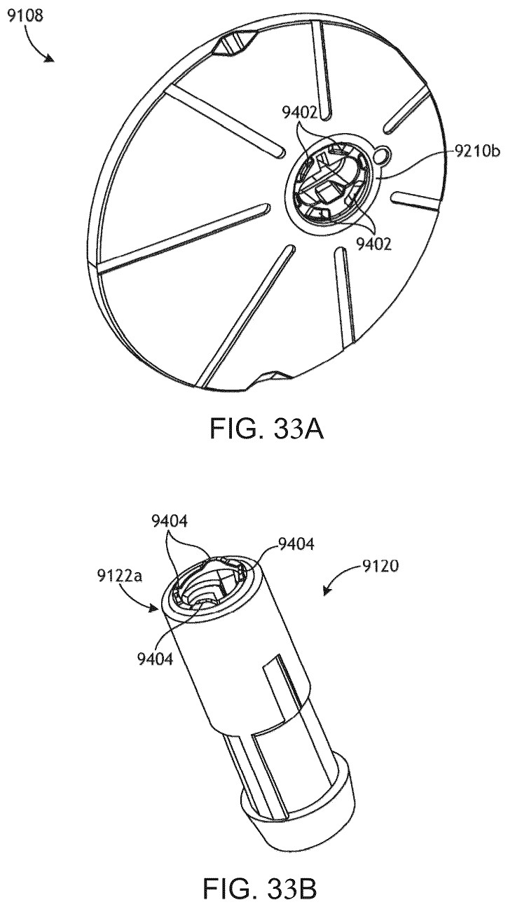

Referring to FIGS. 7S and 7T for purpose of illustration and not limitation, in accordance with the disclosed subject matter, cap 20708 can include one or more surfaces to engage other elements in the applicator 20150 to provide support or limit movement in the case of a shock event, for example, a drop. For example, the cap can include a sheath support surface 20708K, configured to support the sheath 20704 during a shock event. The sheath support surface 20708K can limit distal movement of the sheath 20704 during a shock event. This can lead to less stress on the sensor carrier 20710 and the sensor control device 20102 and can reduce the risk of the sensor control device 20102 dislodging form the sensor carrier 20710. Additionally, or alternatively, cap 20708 can include a raised ridge 20708L. The raised ridge 20708L can interface with a plug, such as an elastomeric plug 9130A (which can, in some embodiments, be coupled to a desiccant cap 9130 and in other embodiments, as can be seen in FIG. 7V, coupled to sensor cap 9120). In some embodiments, the elastomeric plug can include liquid silicone rubber. The raised ridge 20708L can thereby also support the sharp carrier 201102, sensor carrier 20710, sensor control device 20102, and accordingly, can prevent dislodging of the sensor control device 20102 from the sensor carrier 20710 during a shock event. Furthermore, additional support on the elastomeric plug 9130A and other features can increase the stress on various seals in the applicator 20150, and thereby improve the seals prior to removing the cap 20708 from the housing. For example, as can be seen in FIG. 7U, elastomeric plug 9130A may include stop surface 9130Aa, seal bead 9130Ab, intermediate surface 9130Ac, and top surface 9130Ad. As can be seen in FIG. 7U, top surface 9130Ad can include a triangular surface with squares or rectangular corners instead of pointed vertices. As can be seen in FIG. 7V, seal bead 9130Ab may be inserted in sensor cap 9120 until stop surface 9130Aa (as shown in cross-sectional view of FIG. 33) mates to the bottom of surface of sensor cap 9120. Seal bead 9130Ab may generally be barrel shaped (i.e., cylindrical shape with a thicker middle portion that tapers towards the top and bottom of seal bead 9130Ab) and configured to create an interference fit 9130Abb between the seal bead 9130Ab and the inner diameter of sensor cap 9120 which creates a sterile barrier and to seal inner chamber 9124.

### Example Embodiments of Applicator Sheaths

FIGS. 8A and 8B are a side view and perspective view, respectively, depicting an example embodiment of sheath 704. In this example embodiment, sheath 704 can stage sensor control device 102 above a user's skin surface prior to application. Sheath 704 can also contain features that help retain a sharp in a position for proper application of a sensor, determine the force required for sensor application, and guide sheath 704 relative to housing 702 during application. Detent snaps 1402 are near a proximal end of sheath 704, described further with respect to FIG. 8C below. Sheath 704 can have a generally cylindrical cross section with a first radius in a proximal section (closer to top of figure) that is shorter than a second radius in a distal section (closer to bottom of figure). Also shown are a plurality of detent clearances 1410, three in the example embodiment. Sheath 704 can include one or more detent clearances 1410, each of which can be a cutout with room for sheath snap lead-in feature 1330 to pass distally into until a distal surface of locking rib 1340 contacts a proximal surface of detent clearance 1410.

Guide rails 1418 are disposed between sensor carrier traveler limiter face 1420 at a proximal end of sheath 704 and a cutout around lock arms 1412. Each guide rail 1418 can be a channel between two ridges where the guide edge 1326 of housing guide rib 1321 can slide distally with respect to sheath 704.

Lock arms 1412 are disposed near a distal end of sheath 704 and can include an attached distal end and a free proximal end, which can include lock arm interface 1416. Lock arms 1412 can lock sensor carrier 710 to sheath 704 when lock arm interface 1416 of lock arms 1412 engage lock interface 1502 of sensor carrier 710. Lock arm strengthening ribs 1414 can be disposed near a central location of each lock arm 1412 and can act as a strengthening point for an otherwise weak point of each lock arm 1412 to prevent lock arm 1412 from bending excessively or breaking.

Detent snap stiffening features 1422 can be located along the distal section of detent snaps 1402 and can provide reinforcement to detent snaps 1402. Alignment notch 1424 can be a cutout near the distal end of sheath 704, which provides an opening for user alignment with sheath orientation feature of platform 808. Stiffening ribs 1426 can include buttresses, that are triangularly shaped here, which provide support for detent base 1436. Housing guide rail clearance 1428 can be a cutout for a distal surface of housing guide rib 1321 to slide during use.

FIG. 8C is a close-up perspective view depicting an example embodiment of detent snap 1402 of sheath 704. Detent snap 1402 can include a detent snap bridge 1408 located near or at its proximal end. Detent snap 1402 can also include a detent snap flat 1406 on a distal side of detent snap bridge 1408. An outer surface of detent snap bridge 1408 can include detent snap rounds 1404 which are rounded surfaces that allow for easier movement of detent snap bridge 1408 across interior surfaces of housing 702 such as, for example, locking rib 1340.

FIG. 8D is a side view depicting an example embodiment of sheath 704. Here, alignment notch 1424 can be relatively close to detent clearance 1410. Detent clearance 1410 is in a relatively proximal location on distal portion of sheath 704.

FIG. 8E is an end view depicting an example embodiment of a proximal end of sheath 704. Here, a back wall for guide rails 1446 can provide a channel to slidably couple with housing guide rib 1321 of housing 702. Sheath rotation limiter 1448 can be notches which reduce or prevent rotation of the sheath 704.

FIGS. 8F-8H are perspective views of an alternative example embodiment of sheath 6704 in various stages of assembly with other components of the applicator. As shown in FIG. 8F, sheath 6704 can have many of the same features as sheath 704, previously described with respect to FIGS. 8A-8C. Sheath 6704, for example, can include one or more detent snaps 6404 having one or more detent rounds 6402 attached thereto. Sheath 6704, however, can be shorter in overall length as compared to sheath 702. In addition, sheath 6704 can include one or more inner sheath ribs 6425 disposed on the inner surface of sheath 6704, and which protrude in an inward direction towards the central axis of sheath 6704.

Turning to FIG. 8G, sheath 6704 is shown in perspective view in a stage of assembly with applicator housing 6702 and sensor carrier 6710. One or more inner sheath ribs 6425 of sheath 6704 can interface with one or more corresponding rib notches 6519 in sensor carrier 6710. The fitted interface between corresponding ribs 6425 and notches 6519 can help maintain axial alignment of the sheath 6704 and sensor carrier 6710 during the sensor insertion process. Furthermore, the interface between ribs 6425 and notches 6519 can reduce lateral and rotational movement between the applicator components, which can, in turn, reduce the chance of improper sensor insertion.

Turning to FIG. 8H, sheath 6704 is shown in perspective view in a stage of assembly with applicator housing 6702 and sensor electronics housing 706, which has been inserted into sensor carrier 6710. Inner sheath ribs 6425 are also shown.

It should be noted that although six inner sheath ribs 6425 and six corresponding rib notches 6519 are depicted, any number of ribs and notches are fully within the scope of the present disclosure. Moreover, while ribs 6425 are depicted with a rounded surface edge, in other embodiments, ribs 6425 can have a rectangular or triangular shape, and rib notches 6519 can have a corresponding receiving shape for interfacing with ribs 6425. In addition, although ribs 6425 are depicted as being disposed on an inner circumferential surface of sheath 6704, ribs 6425 can also be disposed on any other surface of sheath 6704, or portion thereof, that comes into contact with sensor carrier 6710.

Referring to FIGS. 81-80, for purpose of illustration and not limitation, a sheath 20704 is provided, in accordance with the disclosed subject matter. Sheath 20704 can be made of Delrin or other suitable materials, for example, other low friction polymers. Sheath 20704 can include one or more of the features described herein with regard to sheaths, wherein similar features can operate as described herein. For example, sheath 20704 can include detent snaps 20704A having a free proximal end, configured to engage the sheath ribs 20702S during firing. FIG. 8J shows a close up of the free proximal end of detent snap 20704A. The detent snaps 20704A can include a round portion 20704B, for engagement with the sheath ribs 20702S and a flat portion 20704C for final lockout on housing 20704 after use. The round portion 20704B can include a parting line mismatch 20704D that can prevent a force spike during firing. The detent snaps 20704A can be coupled to the sheath 20704 at an enlarged distal portion 20704E, which can provide support to the detent snap 20704. Sheath 20704 can include a plurality of detent snaps 20704A, for example, three. The sheath 20704 can include one or more, for example, three, housing clearances 20704F, which can allow the sheath 20704 to clear the housing 20702 at the end of firing. In accordance with the disclosed subject matter, sheath 20704 can further include a plurality of stiffening ribs 20704P (e.g., six) which can stiffen the sheath 20704.

Sheath 20704 can include a plurality of guides 20704G for engaging the sheath guide rails 20702J of the housing 20702. Sheath 20704 can further include a slot 20704H including a stop 20704I at a distal end of the slot 20704H configured to engage the sheath guide rails 20702J of the sheath 20702 to limit further proximal movement of the sheath 20704 relative the housing 20702 at the end of firing. Sheath 20704 can also include a clearance 20704T for clearing the sensor carrier biasing feature 20702I disposed on the sheath guide rails 20702J of the housing 20702.

In accordance with the disclosed subject matter, sheath 20704 can include lock arms 20704J. Lock arms 20704J can be configured to engage the sensor carrier 20710 and limit movement of the sensor carrier 20710 or sheath 20704 prior to firing. The lock arms 20704J can include a free proximal end 20704K and an attached distal end 20704L. The free proximal end 20704K can include a lock arm interface 20704M disposed on an inner surface of the lock arm 20704J. The lock arm interface 20704M can engage a lock ledge 20710N on the sensor carrier 20710. For example, when cap 20708 is coupled to housing 20702, the cap 20708 can urge the lock arm 20704J inwardly and can cause the lock arm interface 20704M to engage the sensor carrier 20710. That is, the lock arms 20704J can wedge between the cap 20708 and the sensor carrier 20710. Accordingly, lock arm 20704J can limit proximal movement of the sheath 20704 when the cap 20708 is coupled to the housing 20702. Such engagement can limit movement of the sheath 20704 during a shock event, such as a drop. The lock arm interface 20704M can have a triangle shape when viewed in side view (e.g., FIG. 8N) and a “U” shape when viewed in top view (e.g., FIG. 8K). The shape of the lock arm interface 20704M can provide benefits during manufacturing. For example, the shape of the lock arm interface 20704M can allow the sheath 20704 to be force ejected from a mold during manufacturing of the sheath 20704. Force ejecting the sheath 20704 can allow for a more simplified manufacture process, for example, using a one-piece mold, and can eliminate parting lines created from two-piece molds. Parting lines can result in an unsmooth surface which can catch on the sensor carrier 20710 during firing and can result in potential spikes in firing force. Accordingly, using force ejection and a one-piece mold can create a smoother lock arm interface 20704M and prevent potential spikes in firing force due to parting lines.

The proximal free end of the lock arm 20704J can further include a sharp edge 20704N on an outer surface. The sharp edge 20704N can be configured to engage crush ribs 20708F disposed on the cap 20708 during a shock event. The sharp edge 20704N can dig into the crush ribs 20708F and permanently deform the crush ribs 20708F, which can absorb energy during a shock event, and prevent sheath 20704 collapse. The shape lock arm interface 20704M can also be beneficial for drop protection. The ramp can force the lock arm 20704J to move radially as the sheath 20704 collapsed during a drop. This can force the sharp edge 20704N to dig into the crush ribs 20708F and can help to stop the sheath 20704 from collapsing. Sheath 20704 can include a plurality of lock arms 20704J, for example, two lock arms 20704J.

Additionally or alternatively, sheath 20704 can include rib 20704U configured to engage a lock interface 20710F on a sensor retention arm 20710B on the sensor carrier 20710. The rib 20704U can prevent the sensor retention arm 20710B from flexing outwardly, for example, during a shock event, and therefore can prevent movement of the sensor control device 20102 during a shock event. Rib 20704U can have a height (i.e., in the longitudinal direction) selected such that even if the sheath 20704 moves proximally or distally during a shock event, the rib 20704U will continue to engage lock interface 20710F on a sensor retention arm 20710B on the sensor carrier 20710 and prevent the sensor control device 20102 from dislodging from the sensor carrier 20710.

Sheath 20704 can include a noise damper 20704O. The noise damper 20704O can be configured to engage the sharp carrier 201102 as the sharp carrier 201102 is retracted to slow movement of the sharp carrier 201102 and can thereby reduce noise produced by the sharp carrier 201102 engaging the sheath 20704. In example embodiments, the noise damper 20704O includes an angled ramp extending from the inner surface of sheath 20704, but other suitable configurations can be used.

In accordance with the disclosed subject matter, sheath 20704 can include a slot 20704Q configured to receive sharp carrier retention feature 20710L disposed on the sensor carrier 20710 and to thereby permit partial retraction of the sharp carrier 201102 during deployment (as described in greater detail below). The sheath 20704 can also include cap lead-in 20704R, alignment notch 20704S and skin interface 20704T.

### Example Embodiments of Sensor Carriers

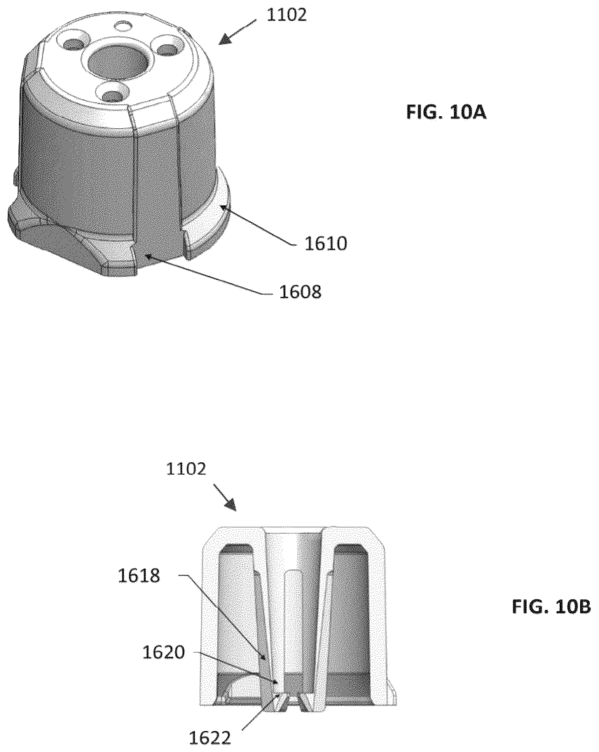

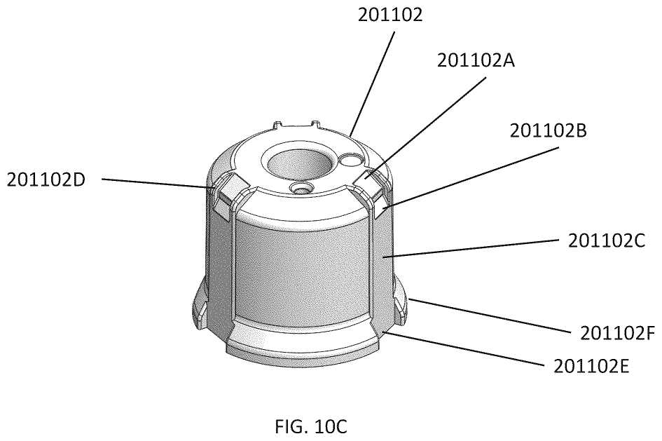

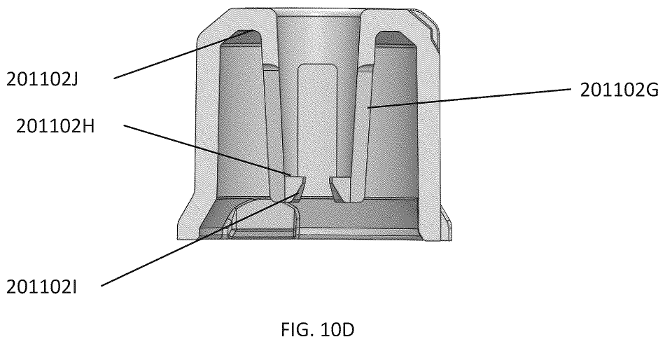

FIG. 9A is a proximal perspective view depicting an example embodiment of sensor carrier 710 that can retain sensor electronics within applicator 150. It can also retain sharp carrier 1102 with sharp module 2500. In this example embodiment, sensor carrier 710 generally has a hollow round flat cylindrical shape and can include one or more deflectable sharp carrier lock arms 1524 (e.g., three) extending proximally from a proximal surface surrounding a centrally located spring alignment ridge 1516 for maintaining alignment of spring 1104. Each lock arm 1524 has a detent or retention feature 1526 located at or near its proximal end. Shock lock 1534 can be a tab located on an outer circumference of sensor carrier 710 extending outward and can lock sensor carrier 710 for added safety prior to firing. Rotation limiter 1506 can be a proximally extending relatively short protrusion on a proximal surface of sensor carrier 710 which limits rotation of carrier 710. Sharp carrier lock arms 1524 can interface with sharp carrier 1102 as described with reference to FIGS. 10A-10E below.

FIG. 9B is a distal perspective view of sensor carrier 710. Here, one or more sensor electronics retention spring arms 1518 (e.g., three) are normally biased towards the position shown and include a detent 1519 that can pass over the distal surface of electronics housing 706 of device 102 when housed within recess or cavity 1521. In certain embodiments, after sensor control device 102 has been adhered to the skin with applicator 150, the user pulls applicator 150 in a proximal direction, i.e., away from the skin. The adhesive force retains sensor control device 102 on the skin and overcomes the lateral force applied by spring arms 1518. As a result, spring arms 1518 deflect radially outwardly and disengage detents 1519 from sensor control device 102 thereby releasing sensor control device 102 from applicator 150.

FIG. 9C is a perspective view of an alternative example embodiment of sensor carrier 6710. As shown in FIG. 9C, sensor carrier 6710 can have many of the same features as sensor carrier 710, previously described with respect to FIGS. 9A-9B. In addition, sensor carrier 6710 also includes one or more notch ribs 6519 disposed along an outer circumferential surface. As best seen in FIGS. 8F-8H, notch ribs 6519 are configured to interface with inner sheath ribs 6425 in order to maintain axial alignment of the sheath and sensor carrier and reduce lateral and rotational movement between applicator components during the sensor insertion process.

Referring to FIGS. 9D and 9E, for purpose of illustration and not limitation, an example embodiment of sensor carrier 20710 is provided. Sensor carrier 20710 can include one or more of the features described herein with regard to sensor carriers, wherein similar features can operate as described herein. For example, sensor carrier 20710 can include a base 20710A and first and second retention arms 20710B. Each retention arm 20710B can include a first end portion 20710C coupled to the base 20710A and a free end portion 20710D. For example, each retention arm 20710B can be coupled to base 20710A at a first half of the base 20710A and the free end portion 20710D can extend toward a second half of the base 20710A. Each retention arm 20710B can include a sensor retention feature 20710E disposed on an inner surface of the sensor retention arm 20710B. The sensor retention feature 20710E can be disposed on the free end portion 20710D. The sensor retention feature 20710E can be configured to retain the sensor control device 20102 within housing 20702. The retention feature 20710E can include a conical surface and angular parting line, which can allow for release of the sensor control device 20102 upon delivery. Each retention arm 20710 can include a lock interface 20710F disposed on an outer surface of the retention arm 20710B. The lock interface 20710F can engage rib 20704U on the sheath 20704. As described hereinabove, the rib 20704U can prevent the sensor retention arm 20710B from flexing outwardly, for example, during a shock event, and therefore can keep retention feature 20710E engaged with the sensor control device 20102, and thereby prevent movement of the sensor control device 20102 during a shock event.

Sensor carrier 20710 can include a plurality of housing attachment features 20710F, for example three housing attachment features 20710F. The housing attachment features 20710F can be equally spaced on the sensor carrier 20710 and can extend upwardly from a top surface of the sensor carrier 20710. Each sensor control device housing attachment feature 20710F can include a housing snap 20710G, housing locator feature 20710H, biasing feature 20710I, and housing stop 20710J. The housing locator feature 20710H can locate the sensor carrier 20710 relative to the housing 20702 when the two are to be coupled together. The housing snap 20710G can engage the sensor carrier attach slots 20702K on the housing 20702 to couple the sensor carrier 20710 to the housing 20702. The biasing feature 207101 can engage sensor carrier biasing feature 20702M on housing 20702 configured to remove slop between the sensor carrier 20710 and the housing 20702. Housing stop 20710J can engage sensor carrier hard stop 20702L on sheath guide rails 20702J on housing 20702 to locate the sensor carrier 20710 axially relative to the housing 20702.

Sensor carrier 20710 can further include a plurality of sharp carrier lock arms 20710K, for example three sharp carrier lock arms 20710K. The sharp carrier lock arms 20710K can be equally spaced on the sensor carrier 20710 and can extend upwardly form a top surface of the sensor carrier 20710. Each sharp carrier lock arm 20710K can include a sharp carrier retention feature 20710L and a rib 20710M. Rib 20710M can engage an inner surface of the sheath 20704, which can urge the sharp carrier lock arm 20710K inwardly and cause sharp carrier retention feature 20710L to retain sharp carrier 201102, as described in greater detail below. The carrier retention feature 20710L can have a triangle shape when viewed in side view and a “U” shape when viewed in top view.

In accordance with the disclosed subject matter, the sensor carrier 20710 can include a plurality of lock ledges 20710N configured to engage lock arm interface 20704M of the sheath 20704 as described herein above. For example, the sensor carrier 20710 can include two lock ledges 20710N. Sensor carrier 20710 can include recesses 20710O disposed proximate each lock ledge 20710N and configured to receive the lock arm interface 20704M during firing, to prevent the lock arm 20704J from engaging with housing 20702 during firing. Sensor carrier 20710 can include a hole 20710P extending through the middle of base 20710A. The hole 20710P can guide and limit movement of sharp hub 205014 during insertion. Additionally, or alternatively, sensor carrier 20710 can include spring locator 20710Q.

A bottom surface of the sensor carrier 20710 can include stiffening ribs 20710R and sensor locator ribs 20710S, which can limit planar motion of the sensor control device 20102 relative the sensor carrier 20710. The bottom surface of the sensor carrier 20710 can include a sensor support surface 20710T configured to support the sensor control device 20102.

### Example Embodiments of Sharp Carriers

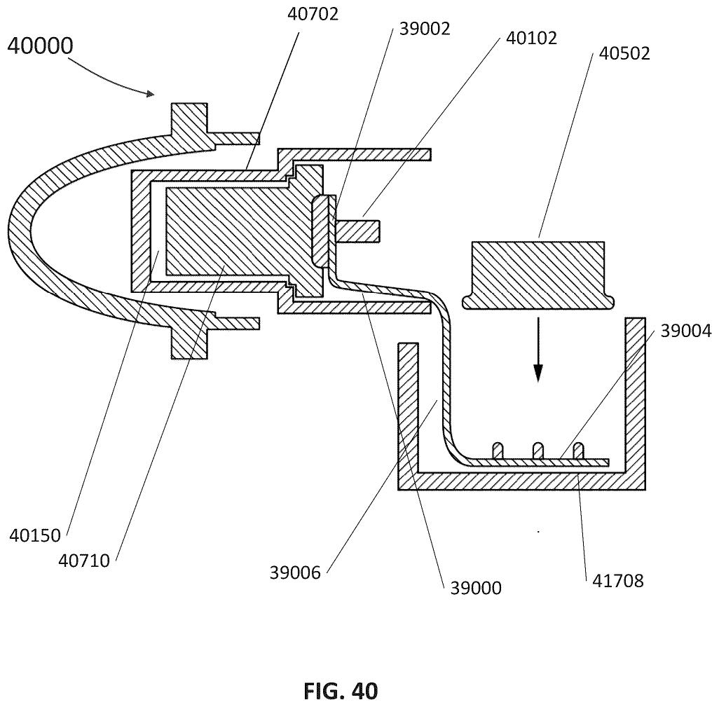

FIGS. 10A and 10B are a proximal perspective view and a side cross-sectional view, respectively, depicting an example embodiment of sharp carrier 1102. Sharp carrier 1102 can grasp and retain sharp module 2500 within applicator 150. It can also automatically retract as a result of one or more springs changing from a preloaded, compressed state to an expanded state during an insertion process, as described with respect to FIGS. 40A-40F. Near a distal end of sharp carrier 1102 can be anti-rotation slots 1608 which prevent sharp carrier 1102 from rotating when located within a central area of sharp carrier lock arms 1524 (as shown in FIG. 9A). Anti-rotation slots 1608 can be located between sections of sharp carrier base chamfer 1610, which can ensure full retraction of sharp carrier 1102 through sheath 704 upon retraction of sharp carrier 1102 at the end of the deployment procedure.

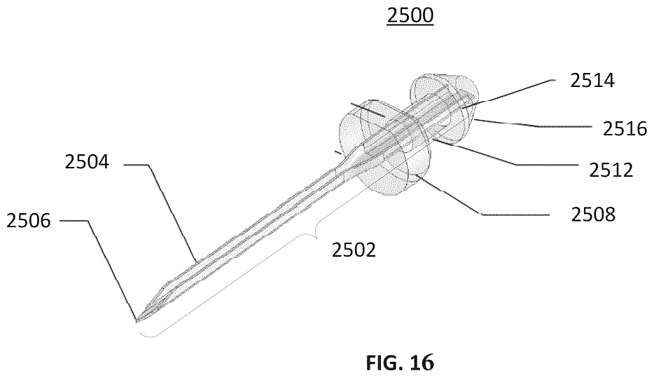

As shown in FIG. 10B, sharp retention arms 1618 can be located in an interior of sharp carrier 1102 about a central axis and can include a sharp retention clip 1620 at a distal end of each arm 1618. Sharp retention clip 1620 can have a proximal surface which can be nearly perpendicular to the central axis and can abut a distally facing surface of sharp hub 2516 (FIG. 16).

Referring to FIGS. 10C and 10D, for purpose of illustration and not limitation, an example embodiment of a sharp carrier 201102 is provided. Sharp carrier 201102 can include one or more features described herein with regard to sharp carriers, wherein similar features can operate as described herein. For example, sharp carrier 201102 can include a series of features for engaging with the three sharp carrier lock arms 20710K of the sensor carrier 20710. The features can include a pre-partial-retraction retention face 201102A and a post-partial-retraction retention face 201102B. The pre-partial retraction retention face 201102A can engage the sharp carrier retention feature 20710L prior to partial retraction, for example, during shipping and storage. Post-partial-retraction retention face 201102B can engage the sharp carrier retention feature 20710L after partial retraction. For example, as the sheath 20704 initially move proximally relative to the sensor carrier 20710, the rib 20710M of the retention arm 20710L can engage slot 20704Q of sheath 20704, which can allow the retention arm 20710L to move radially outward and allow sharp carrier retention feature 20710L to clear the pre-partial retraction retention face 201102A and engage the post-partial retraction retention face 201102B. A height between the end of the pre-partial-retraction face 201102A and the start of the post-partial-retraction face 201102B can be the distance of the partial retraction. A running face 201102C can be disposed below the post-partial-retraction retention face 201102B and can slide against the retention arm 20710L as the sharp carrier 201102 is retracted. Alignment walls 201102D can help to keep the sharp carrier 201102 aligned with the sensor carrier 20704 during partial retraction. Sharp carrier 201102 can include a chamfer 201102F, which can include anti-rotation slots 201102E to engage the retention arms 20710L on the sensor carrier 20710.

Internally, sharp carrier 201102 can include sharp retention arms 201102G including lead-in face 2011021 and sharp hub contact face 201102H. The retention arms 201102G can receive and hold sharp hub 205014. Spring stop 201102J can engage retraction spring 205612.

Spring 205612 can include any type of spring known in the art, such as a helical spring. For example, according to certain embodiments, spring 205612 may include a helical spring constructed of stainless steel. Spring 205612 may include a spring constant of any suitable range, and a wire diameter, an inner diameter, an outer diameter, and a maximum solid strength of any suitable dimension. For example, the spring constant can be about 0.12, the wire diameter can be about 0.65 millimeters, the inner diameter can be about 9.6 millimeter, the outer diameter can be about 11.1 millimeters, and the maximum solid strength can be 11 millimeters. It is noted that each of the parameter values listed above may be provided independently without requiring a particular embodiment of the spring to fulfill all of the listed parameter values. For example, a spring 205612 may be provided having a spring constant of about 0.12 while having different dimensions to those listed above, or a spring 205612 may have a different wire diameter while having the other highlighted dimensions.

### Example Embodiments of Sensor Modules

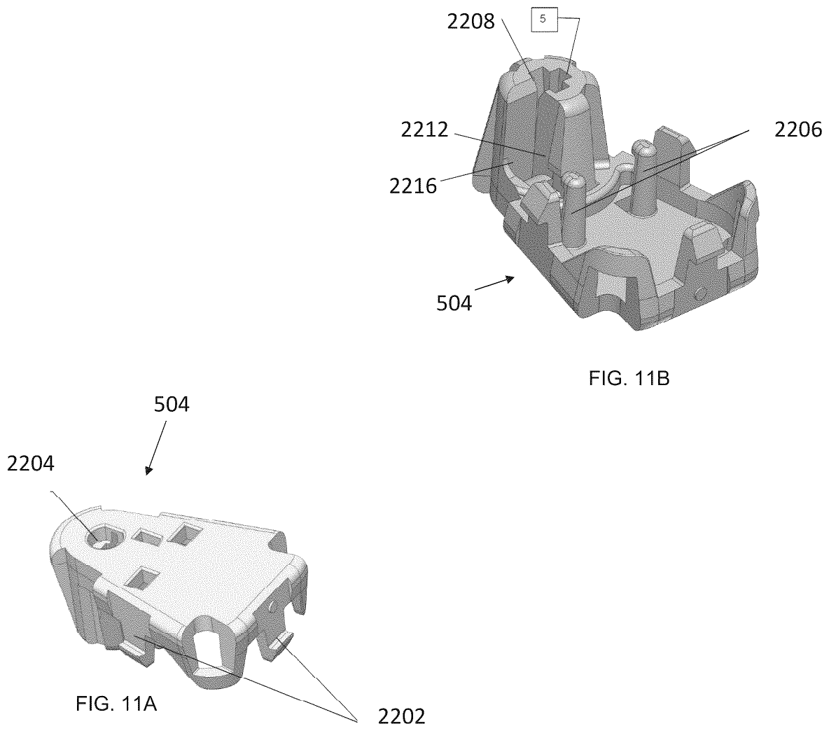

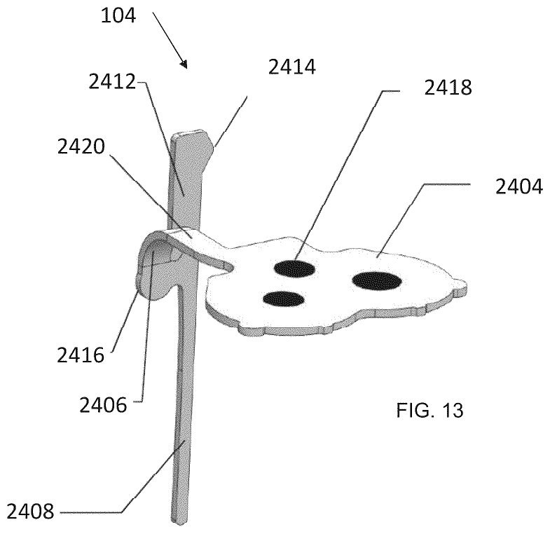

FIGS. 11A and 11B are a top perspective view and a bottom perspective view, respectively, depicting an example embodiment of sensor module 504. Module 504 can hold a connector 2300 (FIGS. 12A and 12B) and a sensor 104 (FIG. 13). Module 504 is capable of being securely coupled with electronics housing 706. One or more deflectable arms or module snaps 2202 can snap into the corresponding features 2010 of housing 706. A sharp slot 2208 can provide a location for sharp tip 2502 to pass through and sharp shaft 2504 to temporarily reside. A sensor ledge 2212 can define a sensor position in a horizontal plane, prevent a sensor from lifting connector 2300 off of posts and maintain sensor 104 parallel to a plane of connector seals. It can also define sensor bend geometry and minimum bend radius. It can limit sensor travel in a vertical direction and prevent a tower from protruding above an electronics housing surface and define a sensor tail length below a patch surface. A sensor wall 2216 can constrain a sensor and define a sensor bend geometry and minimum bend radius.

FIGS. 12A and 12B are perspective views depicting an example embodiment of connector 2300 in an open state and a closed state, respectively. Connector 2300 can be made of silicone rubber that encapsulates compliant carbon impregnated polymer modules that serve as electrical conductive contacts 2302 between sensor 104 and electrical circuitry contacts for the electronics within housing 706. The connector can also serve as a moisture barrier for sensor 104 when assembled in a compressed state after transfer from a container to an applicator and after application to a user's skin. A plurality of seal surfaces 2304 can provide a watertight seal for electrical contacts and sensor contacts. One or more hinges 2208 can connect two distal and proximal portions of connector 2300.

FIG. 13 is a perspective view depicting an example embodiment of sensor 104. A neck 2406 can be a zone which allows folding of the sensor, for example ninety degrees. A membrane on tail 2408 can cover an active analyte sensing element of sensor 104. Tail 2408 can be the portion of sensor 104 that resides under a user's skin after insertion. A flag 2404 can contain contacts and a sealing surface. A biasing tower 2412 can be a tab that biases the tail 2408 into sharp slot 2208. A bias fulcrum 2414 can be an offshoot of biasing tower 2412 that contacts an inner surface of a needle to bias a tail into a slot. A bias adjuster 2416 can reduce a localized bending of a tail connection and prevent sensor trace damage. Contacts 2418 can electrically couple the active portion of the sensor to connector 2300. A service loop 2420 can translate an electrical path from a vertical direction ninety degrees and engage with sensor ledge 2212 (FIG. 11B).

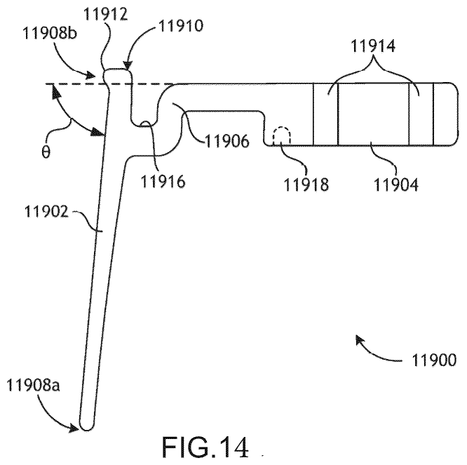

FIG. 14 is a side view of an example sensor 11900, according to one or more embodiments of the disclosure. The sensor 11900 may be similar in some respects to any of the sensors described herein and, therefore, may be used in an analyte monitoring system to detect specific analyte concentrations. As illustrated, the sensor 11900 includes a tail 11902, a flag 11904, and a neck 11906 that interconnects the tail 11902 and the flag 11904. The tail 11902 includes an enzyme or other chemistry or biologic and, in some embodiments, a membrane may cover the chemistry. In use, tail 11902 is transcutaneously received beneath a user's skin, and the chemistry included thereon helps facilitate analyte monitoring in the presence of bodily fluids.

The tail 11902 may be received within a hollow or recessed portion of a sharp (not shown) to at least partially circumscribe the tail 11902 of the sensor 11900. As illustrated, the tail 11902 may extend at an angle Q offset from horizontal. In some embodiments, the angle Q may be about 85°. Accordingly, in contrast to other sensor tails, tail 11902 may not extend perpendicularly from the flag 11904, but instead at an angle offset from perpendicular. This may prove advantageous in helping maintain the tail 11902 within the recessed portion of the sharp.

The tail 11902 includes a first or bottom end 11908a and a second or top end 11908b opposite the bottom end 11908a. A tower 11910 may be provided at or near the top end 11908b and may extend vertically upward from the location where the neck 11906 interconnects the tail 11902 to the flag 11904. During operation, if the sharp moves laterally, the tower 11910 will help pivot the tail 11902 toward the sharp and otherwise stay within the recessed portion of the sharp. Moreover, in some embodiments, tower 11910 may provide or otherwise define a protrusion 11912 that extends laterally therefrom. When the sensor 11900 is mated with the sharp and the tail 11902 extends within the recessed portion of the sharp, the protrusion 11912 may engage the inner surface of the recessed portion. In operation, the protrusion 11912 may help keep the tail 11902 within the recessed portion.

The flag 11904 may comprise a generally planar surface having one or more sensor contacts 11914 arranged thereon. The sensor contact(s) 11914 may be configured to align with a corresponding number of compliant carbon impregnated polymer modules encapsulated within a connector.

In some embodiments, as illustrated, the neck 11906 may provide or otherwise define a dip or bend 11916 extending between the flag 11904 and the tail 11902. The bend 11916 may prove advantageous in adding flexibility to the sensor 11900 and helping prevent bending of the neck 11906.

In some embodiments, a notch 11918 (shown in dashed lines) may optionally be defined in the flag near the neck 11906. The notch 11918 may add flexibility and tolerance to the sensor 11900 as the sensor 11900 is mounted to the mount. More specifically, the notch 11918 may help take up interference forces that may occur as the sensor 11900 is mounted within the mount.

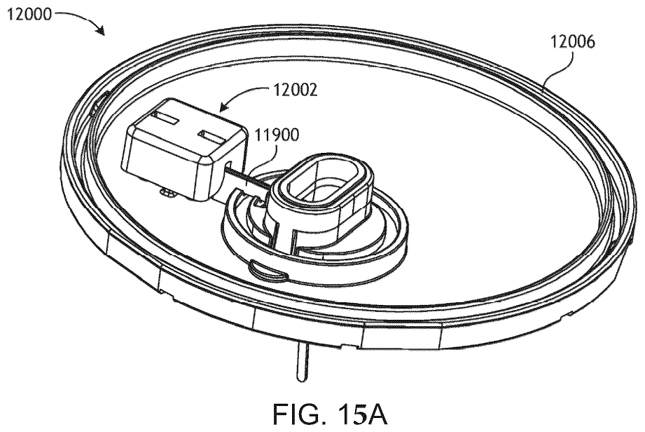

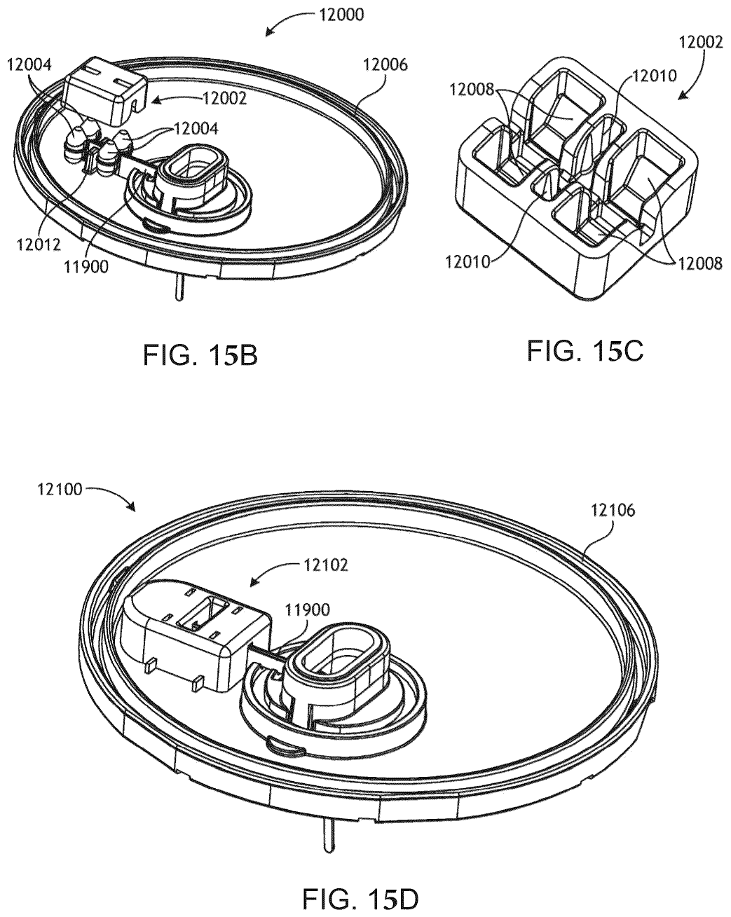

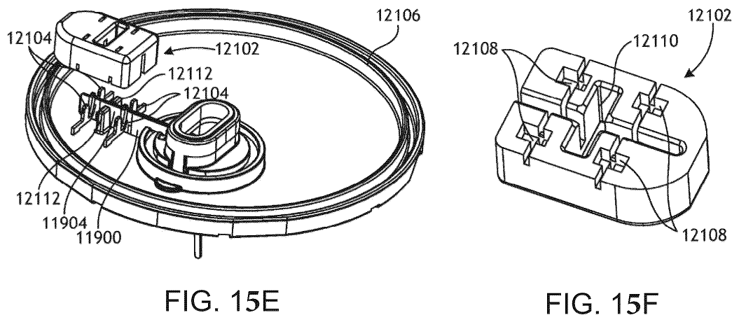

FIGS. 15A and 15B are isometric and partially exploded isometric views of an example connector assembly 12000, according to one or more embodiments. As illustrated, the connector assembly 12000 may include a connector 12002. The connector 12002 may comprise an injection molded part used to help secure one or more compliant carbon impregnated polymer modules 12004 (four shown in FIG. 15B) to a mount 12006. More specifically, the connector 12002 may help secure the modules 12004 in place adjacent the sensor 11900 and in contact with the sensor contacts 11914 (FIG. 14) provided on the flag 11904 (FIG. 14). The modules 12004 may be made of a conductive material to provide conductive communication between the sensor 11900 and corresponding circuitry contacts (not shown) provided within the mount 12006.

As best seen in FIG. 15C, the connector 12002 may define pockets 12008 sized to receive the modules 12004. Moreover, in some embodiments, the connector 12002 may further define one or more depressions 12010 configured to mate with one or more corresponding flanges 12012 (FIG. 15B) on the mount 12006. Mating the depressions 12010 with the flanges 12012 may secure the connector 12002 to the mount 12006 via an interference fit or the like. In other embodiments, the connector 12002 may be secured to the mount 12006 using an adhesive or via sonic welding.

FIGS. 15D and 15E are isometric and partially exploded isometric views of another example connector assembly 12100, according to one or more embodiments. As illustrated, the connector assembly 12100 may include a connector 12102, and FIG. 15F is an isometric bottom view of the connector 12102. The connector 12102 may comprise an injection molded part used to help keep one or more compliant metal contacts 12104 (four shown in FIG. 15E) secured against the sensor 11900 on a mount 12106. More specifically, the connector 12102 may help secure the contacts 12104 in place adjacent to the sensor 11900 and in contact with the sensor contacts 11914 (FIG. 14) provided on the flag 11904. Contacts 12104 may be made of a stamped conductive material that provides conductive communication between the sensor 11900 and corresponding circuitry contacts (not shown) provided within the mount 12106. In some embodiments, for example, the contacts 12104 may be soldered to a PCB (not shown) arranged within the mount 12106.

As best seen in FIG. 15F, the connector 12102 may define pockets 12108 sized to receive the contacts 12104. Moreover, in some embodiments, the connector 12102 may further define one or more depressions 12110 configured to mate with one or more corresponding flanges 12112 (FIG. 15E) on mount 12006. Mating the depressions 12110 with the flanges 12112 may help secure the connector 12102 to the mount 12106 via an interference fit or the like. In other embodiments, the connector 12102 may be secured to the mount 12106 using an adhesive or via sonic welding.

### Example Embodiments of Sharp Modules

FIG. 16 is a perspective view depicting an example embodiment of sharp module 2500 prior to assembly within sensor module 504 (FIG. 6B). Sharp 2502 can include a distal tip 2506 which can penetrate the skin while carrying sensor tail in a hollow or recess of sharp shaft 2504 to put the active surface of the sensor tail into contact with bodily fluid. A hub push cylinder 2508 can provide a surface for a sharp carrier to push during insertion. A hub small cylinder 2512 can provide a space for the extension of sharp hub contact faces 1622 (FIG. 10B). A hub snap pawl locating cylinder 2514 can provide a distal-facing surface of hub snap pawl 2516 for sharp hub contact faces 1622 to abut. A hub snap pawl 2516 can include a conical surface that opens clip 1620 during installation of sharp module 2500.

### Example Embodiments of Applicators and Sensor Control Devices for One Piece Architectures

Referring briefly again to FIGS. 1 and 3A-3G, for the two-piece architecture system, the sensor tray 810 and the sensor applicator 150 are provided to the user as separate packages, thus requiring the user to open each package and finally assemble the system. In some applications, the discrete, sealed packages allow the sensor tray 810 and the sensor applicator 150 to be sterilized in separate sterilization processes unique to the contents of each package and otherwise incompatible with the contents of the other. More specifically, the sensor tray 810, which includes the plug assembly 207, including the sensor 110 and the sharp 220, may be sterilized using radiation sterilization, such as electron beam (or “e-beam”) irradiation. Radiation sterilization, however, can damage the electrical components arranged within the electronics housing of the sensor control device 102. Consequently, if the sensor applicator 150, which contains the electronics housing of the sensor control device 102, needs to be sterilized, it may be sterilized via another method, such as gaseous chemical sterilization using, for example, ethylene oxide. Gaseous chemical sterilization, however, can damage the enzymes or other chemistry and biologies included on sensor 110. Because of this sterilization incompatibility, the sensor tray 810 and the sensor applicator 150 are commonly sterilized in separate sterilization processes and subsequently packaged separately, which requires the user to finally assemble the components for use.

According to embodiments of the present disclosure, the sensor control device 102 may be modified to provide a one-piece architecture that may be subjected to sterilization techniques specifically designed for a one-piece architecture sensor control device. A one-piece architecture allows the sensor applicator 150 and the sensor control device 102 to be shipped to the user in a single, sealed package that does not require any final user assembly steps. Rather, the user needs only open one package and subsequently deliver the sensor control device 102 to the target monitoring location. The one-piece system architecture described herein may prove advantageous in eliminating component parts, various fabrication process steps, and user assembly steps. As a result, packaging and waste are reduced, and the potential for user error or contamination to the system is mitigated.

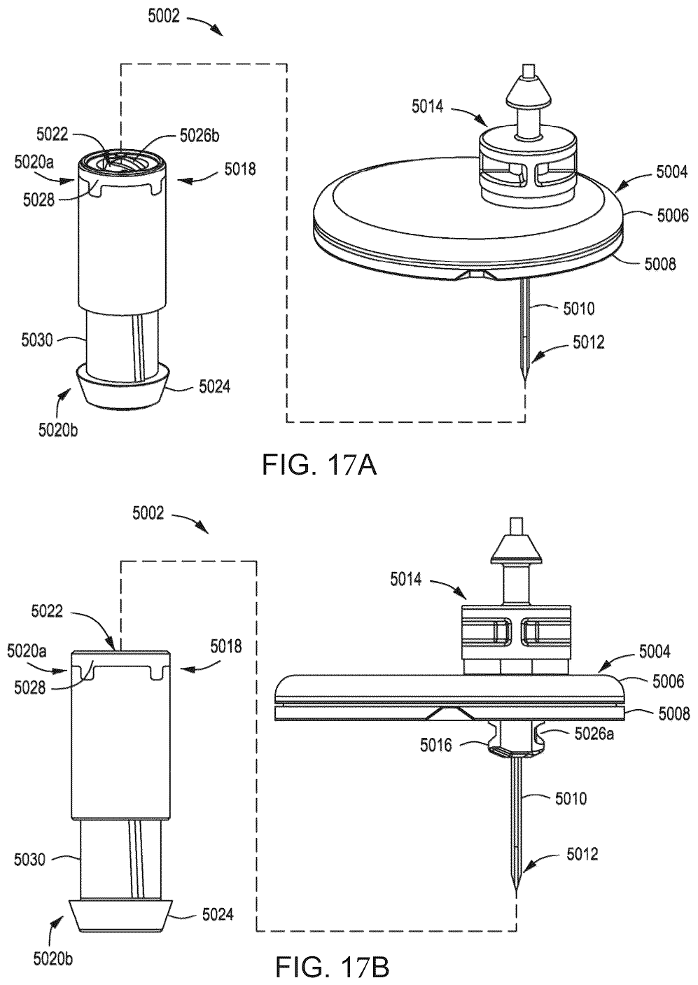

FIGS. 17A and 17B are isometric and side views, respectively, of another example sensor control device 5002, according to one or more embodiments of the present disclosure. The sensor control device 5002 may be similar in some respects to the sensor control device 102 of FIG. 1 and therefore may be best understood with reference thereto. Moreover, the sensor control device 5002 may replace the sensor control device 102 of FIG. 1 and, therefore, may be used in conjunction with the sensor applicator 150 of FIG. 1, which may deliver the sensor control device 5002 to a target monitoring location on a user's skin.

Unlike the sensor control device 102 of FIG. 1, however, the sensor control device 5002 may comprise a one-piece system architecture not requiring a user to open multiple packages and finally assemble the sensor control device 5002 prior to application. Rather, upon receipt by the user, the sensor control device 5002 may already be fully assembled and properly positioned within sensor applicator 150 (FIG. 1). To use the sensor control device 5002, the user need only open one barrier (e.g., the applicator cap 708 of FIG. 3B) before promptly delivering the sensor control device 5002 to the target monitoring location for use.

As illustrated, the sensor control device 5002 includes an electronics housing 5004 that is generally disc-shaped and may have a circular cross-section. In other embodiments, however, the electronics housing 5004 may exhibit other cross-sectional shapes, such as ovoid or polygonal, without departing from the scope of the disclosure. The electronics housing 5004 may be configured to house or otherwise contain various electrical components used to operate the sensor control device 5002. In at least one embodiment, an adhesive patch (not shown) may be arranged at the bottom of the electronics housing 5004. The adhesive patch may be similar to the adhesive patch 105 of FIG. 1 and may thus help adhere the sensor control device 5002 to the user's skin for use.

As illustrated, the sensor control device 5002 includes an electronics housing 5004 that includes a shell 5006 and a mount 5008 that is mateable with the shell 5006. The shell 5006 may be secured to the mount 5008 via a variety of ways, such as a snap fit engagement, an interference fit, sonic welding, one or more mechanical fasteners (e.g., screws), a gasket, an adhesive, or any combination thereof. In some cases, shell 5006 may be secured to the mount 5008 such that a sealed interface is generated therebetween.

The sensor control device 5002 may further include a sensor 5010 (partially visible) and a sharp 5012 (partially visible), used to help deliver the sensor 5010 transcutaneously under a user's skin during application of the sensor control device 5002. As illustrated, corresponding portions of the sensor 5010 and the sharp 5012 extend distally from the bottom of the electronics housing 5004 (e.g., the mount 5008). The sharp 5012 may include a sharp hub 5014 configured to secure and carry the sharp 5012. As best seen in FIG. 17B, sharp hub 5014 may include or otherwise define a mating member 5016. To couple the sharp 5012 to the sensor control device 5002, the sharp 5012 may be advanced axially through the electronics housing 5004 until the sharp hub 5014 engages an upper surface of the shell 5006 and the mating member 5016 extends distally from the bottom of the mount 5008. As the sharp 5012 penetrates the electronics housing 5004, the exposed portion of the sensor 5010 may be received within a hollow or recessed (arcuate) portion of the sharp 5012. The remaining portion of the sensor 5010 is arranged within the interior of the electronics housing 5004.

The sensor control device 5002 may further include a sensor cap 5018, shown exploded or detached from the electronics housing 5004 in FIGS. 17A-17B. The sensor cap 5016 may be removably coupled to the sensor control device 5002 (e.g., the electronics housing 5004) at or near the bottom of mount 5008. The sensor cap 5018 may help provide a sealed barrier that surrounds and protects the exposed portions of the sensor 5010 and the sharp 5012 from gaseous chemical sterilization. As illustrated, the sensor cap 5018 may comprise a generally cylindrical body having a first end 5020a and a second end 5020b opposite the first end 5020a. The first end 5020a may be open to provide access into an inner chamber 5022 defined within the body. In contrast, the second end 5020b may be closed and may provide or otherwise define an engagement feature 5024. As described herein, the engagement feature 5024 may help mate the sensor cap 5018 to the cap (e.g., the applicator cap 708 of FIG. 3B) of a sensor applicator (e.g., the sensor applicator 150 of FIGS. 1 and 3A-3G), and may help remove the sensor cap 5018 from the sensor control device 5002 upon removing the cap from the sensor applicator.

The sensor cap 5018 may be removably coupled to the electronics housing 5004 at or near the bottom of mount 5008. More specifically, the sensor cap 5018 may be removably coupled to the mating member 5016, which extends distally from the bottom of mount 5008. In at least one embodiment, for example, the mating member 5016 may define a set of external threads 5026a (FIG. 17B) mateable with a set of internal threads 5026b (FIG. 17A) defined by the sensor cap 5018. In some embodiments, the external and internal threads 5026a, b may comprise a flat thread design (e.g., lack of helical curvature), which may prove advantageous in molding the parts. Alternatively, the external and internal threads 5026a, b may comprise a helical threaded engagement. Accordingly, the sensor cap 5018 may be threadably coupled to the sensor control device 5002 at the mating member 5016 of the sharp hub 5014. In other embodiments, the sensor cap 5018 may be removably coupled to the mating member 5016 via other types of engagements including, but not limited to, an interference or friction fit, or a frangible member or substance that may be broken with minimal separation force (e.g., axial or rotational force).

In some embodiments, the sensor cap 5018 may comprise a monolithic (singular) structure extending between the first and second ends 5020a, b. In other embodiments, however, the sensor cap 5018 may comprise two or more component parts. In the illustrated embodiment, for example, the sensor cap 5018 may include a seal ring 5028 positioned at the first end 5020a and a desiccant cap 5030 arranged at the second end 5020b. The seal ring 5028 may be configured to help seal the inner chamber 5022, as described in more detail below. In at least one embodiment, the seal ring 5028 may comprise an elastomeric O-ring. The desiccant cap 5030 may house or comprise a desiccant to help maintain preferred humidity levels within the inner chamber 5022. The desiccant cap 5030 may also define or otherwise provide the engagement feature 5024 of the sensor cap 5018.

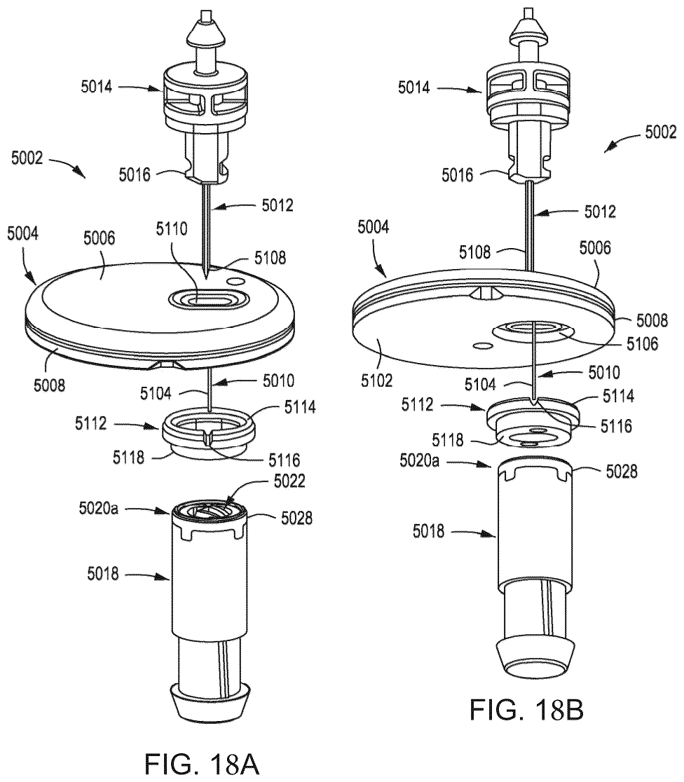

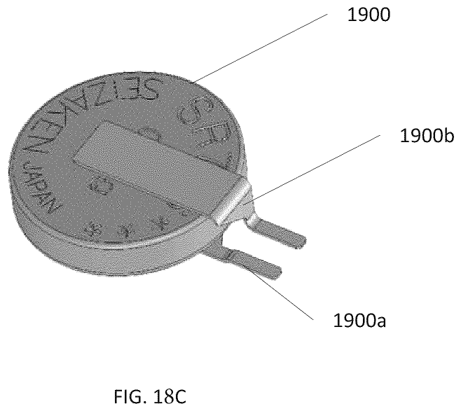

FIGS. 18A and 18B are exploded isometric top and bottom views, respectively, of the sensor control device 5002, according to one or more embodiments. The shell 5006 and the mount 5008 operate as opposing clamshell halves that enclose or otherwise substantially encapsulate various electronic components of the sensor control device 5002. More specifically, electronic components may include, but are not limited to, a printed circuit board (PCB), one or more resistors, transistors, capacitors, inductors, diodes, and switches. A data processing unit and a battery may be mounted to or otherwise interact with the PCB. The data processing unit may comprise, for example, an application specific integrated circuit (ASIC) configured to implement one or more functions or routines associated with operation of the sensor control device 5002. More specifically, the data processing unit may be configured to perform data processing functions, where such functions may include, but are not limited to, filtering and encoding of data signals, each of which corresponds to a sampled analyte level of the user. The data processing unit may also include or otherwise communicate with an antenna for communicating with the reader device 120 (FIG. 1). The battery may provide power to the sensor control device 5002 and, more particularly, to the electronic components of the PCB. For example, battery can be any battery known to a person of ordinary skill in the art, such as a coin cell battery or a button battery, as shown in FIG. 18C. In certain embodiments, battery 1900 may include silver oxide batteries. Battery 1900 may be laser welded to the PCB and located to ensure that outer diameter of battery remains within outer perimeter of PCB. In some embodiments, battery 1900 may be connected to PCB using negative battery tab 1900a and positive battery tab 1900b. For example, negative battery tab 1900a of battery 1900 may be substantially planar and in the same plane as negative terminal of battery 1900; while positive battery tab 1900b may include one or more bends so that one end of positive terminal 1900b is in contact with and in the same plane as positive battery terminal while a second end configured to couple to the PCB is in the same plane as the negative battery tab 1900a. Furthermore, battery 1900 may be positioned in a battery aperture on the PCB to eliminate electrical interference between negative battery tab 1900a and PCB. While not shown, the sensor control device 5002 may also include an adhesive patch that may be applied to the bottom 5102 (FIG. 18B) of the mount 5008 and may help adhere the sensor control device 5002 to the user's skin for use.

It will be understood by those of skill in the art that the battery assembly embodiments described herein, including the PCB and data processing unit, may be implemented in other medical devices, including sensor control devices having other types of housing and configurations relating thereto. In other words, the example embodiments of sensor control device 5002, comprising opposing clamshell halves, are intended to be non-limiting and merely illustrate one type of device that can be used with the battery assembly embodiments described herein.

The sensor control device 5002 may provide or otherwise include a sealed

subassembly that includes, among other component parts, the shell 5006, the sensor 5010, the sharp 5012, and the sensor cap 5018. The sealed subassembly of the sensor control device 5002 may help isolate the sensor 5010 and the sharp 5012 within the inner chamber 5022 (FIG. 18A) of the sensor cap 5018 during a gaseous chemical sterilization process, which might otherwise adversely affect the chemistry provided on the sensor 5010.

The sensor 5010 may include a tail 5104 that extends out an aperture 5106 (FIG. 18B) defined in the mount 5008 to be transcutaneously received beneath a user's skin. The tail 5104 may have an enzyme or other chemistry included thereon to help facilitate analyte monitoring. The sharp 5012 may include a sharp tip 5108 extendable through an aperture 5110 (FIG. 18A) defined by the shell 5006, and the aperture 5110 may be coaxially aligned with the aperture 5106 of the mount 5008. As the sharp tip 5108 penetrates the electronics housing 5004, the tail 5104 of the sensor 5010 may be received within a hollow or recessed portion of the sharp tip 5108. The sharp tip 5108 may be configured to penetrate the skin while carrying the tail 5104 to put the active chemistry of the tail 5104 into contact with bodily fluids.

The sharp tip 5108 may be advanced through the electronics housing 5004 until the sharp hub 5014 engages an upper surface of the shell 5006 and the mating member 5016 extends out the aperture 5106 in the bottom 5102 of mount 5008. In some embodiments, a seal member (not shown), such as an O-ring or seal ring, may interpose the sharp hub 5014 and the upper surface of the shell 5006 to help seal the interface between the two components. In some embodiments, the seal member may comprise a separate component part, but may alternatively form an integral part of the shell 5006, such as being a co-molded or overmolded component part.

The sealed subassembly may further include a collar 5112 that is positioned within the electronics housing 5004 and extends at least partially into the aperture 5106. The collar 5112 may be a generally annular structure that defines or otherwise provides an annular ridge 5114 on its top surface. In some embodiments, as illustrated, a groove 5116 may be defined in the annular ridge 5114 and may be configured to accommodate or otherwise receive a portion of the sensor 5010 extending laterally within the electronics housing 5004.

In assembling the sealed subassembly, bottom 5118 of the collar 5112 may be exposed at the aperture 5106 and may sealingly engage the first end 5020a of the sensor cap 5018 and, more particularly, the seal ring 5028. In contrast, the annular ridge 5114 at the top of the collar 5112 may sealingly engage an inner surface (not shown) of the shell 5006. In at least one embodiment, a seal member (not shown) may interpose the annular ridge 5114 and the inner surface of the shell 5006 to form a sealed interface. In such embodiments, the seal member may also extend (flow) into the groove 5116 defined in the annular ridge 5114 and thereby seal about the sensor 5010 extending laterally within the electronics housing 5004. The seal member may comprise, for example, an adhesive, a gasket, or an ultrasonic weld, and may help isolate the enzymes and other chemistry included on the tail 5104.

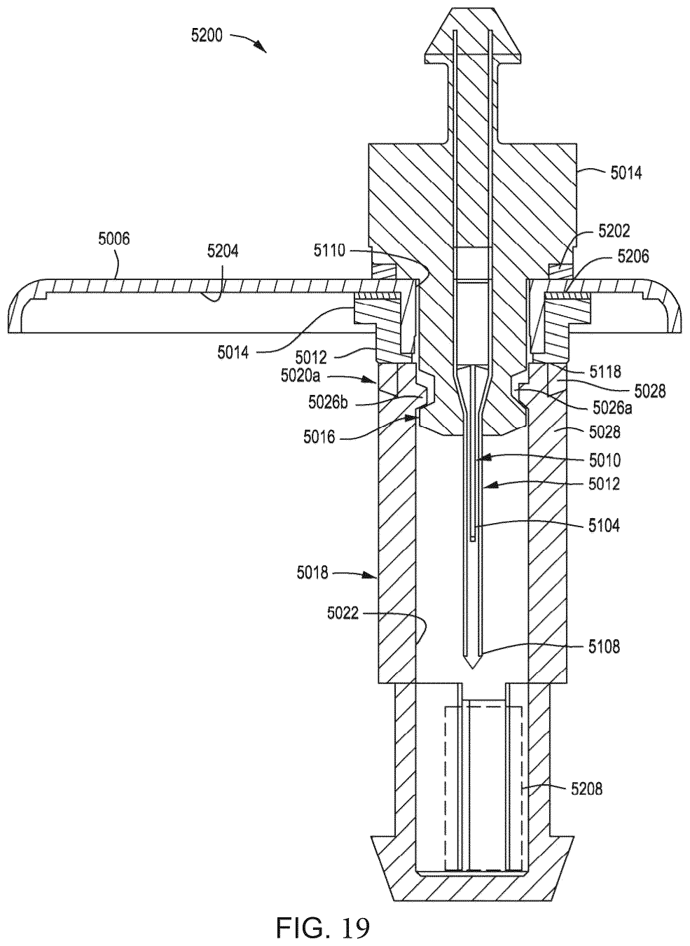

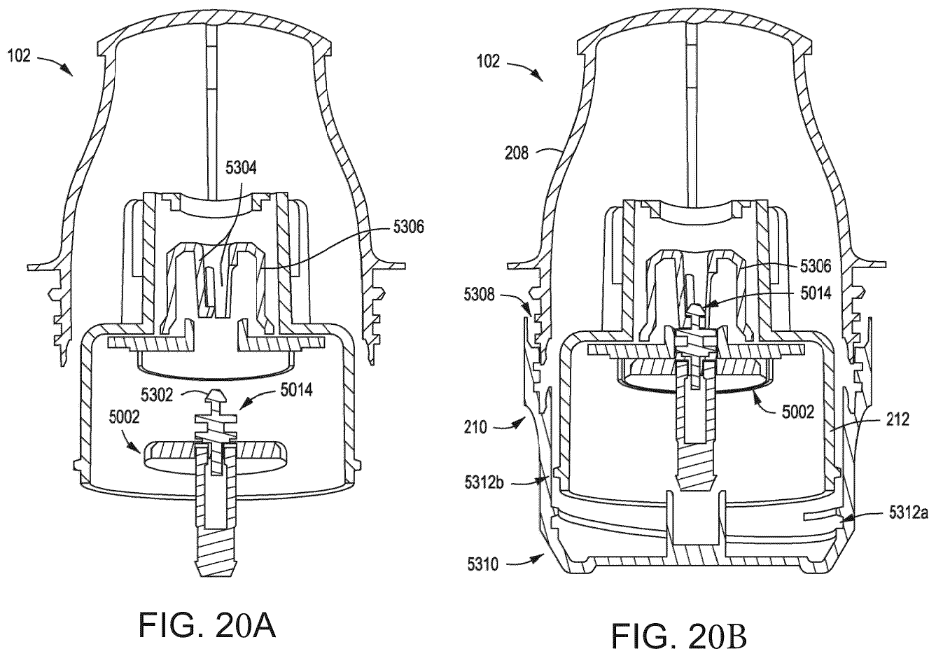

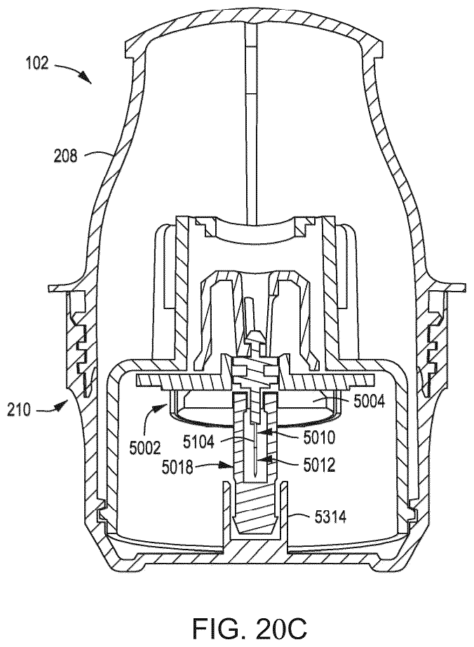

FIG. 19 is a cross-sectional side view of an assembled sealed subassembly 5200, according to one or more embodiments. The sealed subassembly 5200 may form part of the sensor control device 5002 of FIGS. 18A-18B and 20A-20B and may include portions of the shell 5006, the sensor 5010, the sharp 5012, the sensor cap 5018, and the collar 5112. The sealed subassembly 5200 may be assembled in a variety of ways. In one assembly process, the sharp 5012 may be coupled to the sensor control device 5002 by extending the sharp tip 5108 through the aperture 5110 defined in the top of the shell 5006 and advancing the sharp 5012 through the shell 5006 until the sharp hub 5014 engages the top of the shell 5006 and the mating member 5016 extends distally from the shell 5006. In some embodiments, as mentioned above, a seal member 5202 (e.g., an O-ring or seal ring) may interpose the sharp hub 5014 and the upper surface of the shell 5006 to help seal the interface between the two components.

The collar 5112 may then be received over (about) the mating member 5016 and advanced toward an inner surface 5204 of the shell 5006 to enable the annular ridge 5114 to engage the inner surface 5204. A seal member 5206 may interpose the annular ridge 5114 and the inner surface 5204 and thereby form a sealed interface. The seal member 5206 may also extend (flow) into the groove 5116 (FIGS. 20A-20B) defined in the annular ridge 5114 and thereby seal about the sensor 5010 extending laterally within the electronics housing 5004 (FIGS. 20A-20B). In other embodiments, however, the collar 5112 may first be sealed to the inner surface 5204 of the shell 5006, following which the sharp 5012 and the sharp hub 5014 may be extended through the aperture 5110, as described above.

The sensor cap 5018 may be removably coupled to the sensor control device 5002 by threadably mating the internal threads 5026b of the sensor cap 5018 with the external threads 5026a of the mating member 5016. Tightening (rotating) the mated engagement between the sensor cap 5018 and the mating member 5016 may urge the first end 5020a of the sensor cap 5018 into sealed engagement with the bottom 5118 of the collar 5112. Moreover, tightening the mated engagement between the sensor cap 5018 and the mating member 5016 may also enhance the sealed interface between the sharp hub 5014 and the top of the shell 5006, and between the annular ridge 5114 and the inner surface 5204 of the shell 5006.

The inner chamber 5022 may be sized and otherwise configured to receive the tail 5104 and the sharp tip 5108. Moreover, the inner chamber 5022 may be sealed to isolate the tail 5104 and the sharp tip 5108 from substances that might adversely interact with the chemistry of the tail 5104. In some embodiments, a desiccant 5208 (shown in dashed lines) may be present within the inner chamber 5022 to maintain proper humidity levels.

Once properly assembled, the sealed subassembly 5200 may be subjected to any of the radiation sterilization processes mentioned herein to properly sterilize the sensor 5010 and the sharp 5012. This sterilization step may be undertaken apart from the remaining portions of the sensor control device (FIGS. 17A-17B and 18A-18B) to prevent damage to sensitive electrical components. The sealed subassembly 5200 may be subjected to radiation sterilization prior to or after coupling the sensor cap 5018 to the sharp hub 5014. When sterilized after coupling the sensor cap 5018 to the sharp hub 5014, the sensor cap 5018 may be made of a material that permits the propagation of radiation therethrough. In some embodiments, the sensor cap 5018 may be transparent or translucent, but can otherwise be opaque, without departing from the scope of the disclosure.

FIGS. 20A-20C are progressive cross-sectional side views showing assembly of the sensor applicator 150 with the sensor control device 5002, according to one or more embodiments. Once the sensor control device 5002 is fully assembled, it may then be loaded into sensor applicator 150. With reference to FIG. 20A, the sharp hub 5014 may include or otherwise define a hub snap pawl 5302 configured to help couple the sensor control device 5002 to the sensor applicator 150. More specifically, the sensor control device 5002 may be advanced into the interior of the sensor applicator 150 and the hub snap pawl 5302 may be received by corresponding arms 5304 of a sharp carrier 5306 positioned within the sensor applicator 150.

In FIG. 20B, the sensor control device 5002 is shown received by the sharp carrier 5306 and, therefore, secured within the sensor applicator 150. Once the sensor control device 5002 is loaded into the sensor applicator 150, the applicator cap 210 may be coupled to the sensor applicator 150. In some embodiments, the applicator cap 210 and the housing 208 may have opposing, mateable sets of threads 5308 that enable the applicator cap 210 to be screwed onto the housing 208 in a clockwise (or counterclockwise) direction and thereby secure the applicator cap 210 to the sensor applicator 150.

As illustrated, the sheath 212 is also positioned within sensor applicator 150, and the sensor applicator 150 may include a sheath locking mechanism 5310 configured to ensure that the sheath 212 does not prematurely collapse during a shock event. In the illustrated embodiment, the sheath locking mechanism 5310 may comprise a threaded engagement between the applicator cap 210 and the sheath 212. More specifically, one or more internal threads 5312a may be defined or otherwise provided on the inner surface of the applicator cap 210, and one or more external threads 5312b may be defined or otherwise provided on the sheath 212. The internal and external threads 5312a, b may be configured to threadably mate as the applicator cap 210 is threaded to the sensor applicator 150 at the threads 5308. The internal and external threads 5312a, b may have the same thread pitch as the threads 5308 that enable the applicator cap 210 to be screwed onto the housing 208.

In FIG. 20C, applicator cap 210 is shown fully threaded (coupled) to the housing 208. As illustrated, the applicator cap 210 may further provide and otherwise define a cap post 5314 centrally located within the interior of the applicator cap 210 and extending proximally from the bottom thereof. The cap post 5314 may be configured to receive at least a portion of the sensor cap 5018 as the applicator cap 210 is screwed onto the housing 208.

With the sensor control device 5002 loaded within the sensor applicator 150 and the applicator cap 210 properly secured, the sensor control device 5002 may then be subjected to a gaseous chemical sterilization configured to sterilize the electronics housing 5004 and any other exposed portions of the sensor control device 5002. Since the distal portions of the sensor 5010 and the sharp 5012 are sealed within the sensor cap 5018, the chemicals used during the gaseous chemical sterilization process are unable to interact with the enzymes, chemistry, and biologics provided on the tail 5104, and other sensor components, such as membrane coatings that regulate analyte influx.

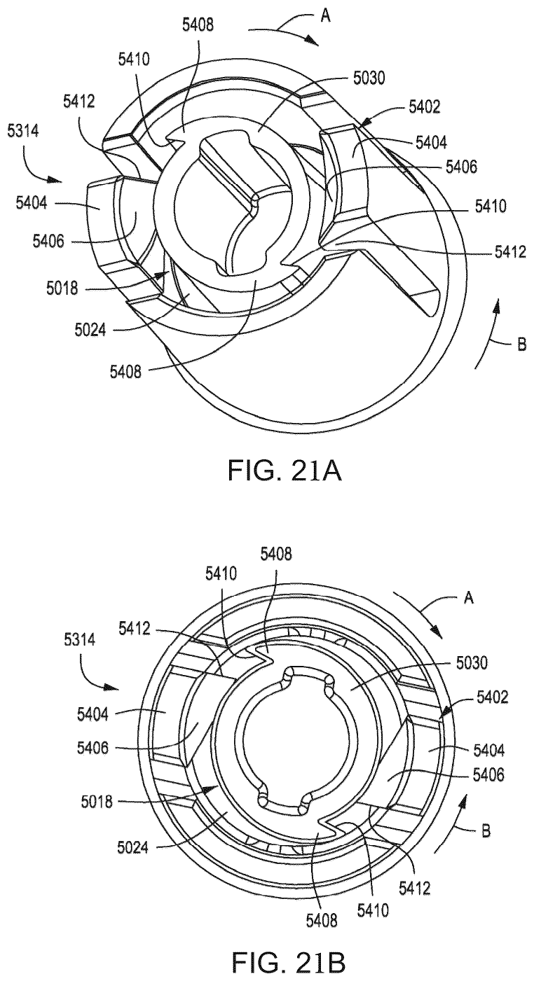

FIGS. 21A and 21B are perspective and top views, respectively, of the cap post 5314, according to one or more additional embodiments. In the illustrated depiction, a portion of the sensor cap 5018 is received within the cap post 5314 and, more specifically, the desiccant cap 5030 of the sensor cap 5018 is arranged within cap post 5314.

As illustrated, the cap post 5314 may define a receiver feature 5402 configured to receive the engagement feature 5024 of the sensor cap 5018 upon coupling (e.g., threading) the applicator cap 210 (FIG. 20C) to the sensor applicator 150 (FIGS. 20A-20C). Upon removing the applicator cap 210 from the sensor applicator 150, however, the receiver feature 5402 may prevent the engagement feature 5024 from reversing direction and thus prevent the sensor cap 5018 from separating from the cap post 5314. Instead, removing the applicator cap 210 from the sensor applicator 150 will simultaneously detach the sensor cap 5018 from the sensor control device 5002 (FIGS. 17A-17B and 20A-20C), and thereby expose the distal portions of the sensor 5010 (FIGS. 20A-20C) and the sharp 5012 (FIGS. 20A-20C).

Many design variations of the receiver feature 5402 may be employed, without departing from the scope of the disclosure. In the illustrated embodiment, the receiver feature 5402 includes one or more compliant members 5404 (two shown) that are expandable or flexible to receive the engagement feature 5024 (FIGS. 17A-17B). The engagement feature 5024 may comprise, for example, an enlarged head and the compliant member(s) 5404 may comprise a collet-type device that includes a plurality of compliant fingers configured to flex radially outward to receive the enlarged head.

The compliant member(s) 5404 may further provide or otherwise define corresponding ramped surfaces 5406 configured to interact with one or more opposing camming surfaces 5408 provided on the outer wall of the engagement feature 5024. The configuration and alignment of the ramped surface(s) 5406 and the opposing camming surface(s) 5408 is such that the applicator cap 210 is able to rotate relative to the sensor cap 5018 in a first direction A (e.g., clockwise), but the cap post 5314 binds against the sensor cap 5018 when the applicator cap 210 is rotated in a second direction B (e.g., counter clockwise). More particularly, as the applicator cap 210 (and thus the cap post 5314) rotates in the first direction A, the camming surfaces 5408 engage the ramped surfaces 5406, which urge the compliant members 5404 to flex or otherwise deflect radially outward and results in a ratcheting effect. Rotating the applicator cap 210 (and thus the cap post 5314) in the second direction B, however, will drive angled surfaces 5410 of the camming surfaces 5408 into opposing angled surfaces 5412 of the ramped surfaces 5406, which results in the sensor cap 5018 binding against the compliant member(s) 5404.

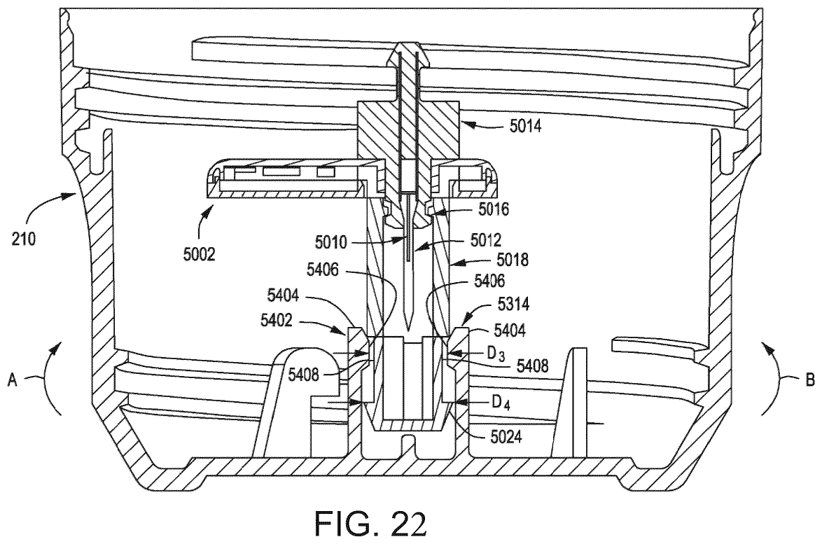

FIG. 22 is a cross-sectional side view of the sensor control device 5002 positioned within the applicator cap 210, according to one or more embodiments. As illustrated, the opening to the receiver feature 5402 exhibits a first diameter D3, while the engagement feature 5024 of the sensor cap 5018 exhibits a second diameter D4 that is larger than the first diameter D3 and greater than the outer diameter of the remaining portions of the sensor cap 5018. As the sensor cap 5018 is extended into the cap post 5314, the compliant member(s) 5404 of the receiver feature 5402 may flex (expand) radially outward to receive the engagement feature 5024. In some embodiments, as illustrated, the engagement feature 5024 may provide or otherwise define an angled or frustoconical outer surface that helps bias the compliant member(s) 5404 radially outward. Once the engagement feature 5024 bypasses the receiver feature 5402, the compliant member(s) 5404 are able to flex back to (or towards) their natural state and thus lock the sensor cap 5018 within the cap post 5314.

As the applicator cap 210 is threaded to (screwed onto) the housing 208 (FIGS. 21A-21C) in the first direction A, the cap post 5314 correspondingly rotates in the same direction and the sensor cap 5018 is progressively introduced into the cap post 5314. As the cap post 5314 rotates, the ramped surfaces 5406 of the compliant members 5404 ratchet against the opposing camming surfaces 5408 of the sensor cap 5018. This continues until the applicator cap 210 is fully threaded onto (screwed onto) the housing 208. In some embodiments, the ratcheting action may occur over two full revolutions of the applicator cap 210 before the applicator cap 210 reaches its final position.

To remove the applicator cap 210, the applicator cap 210 is rotated in the second direction B, which correspondingly rotates the cap post 5314 in the same direction and causes the camming surfaces 5408 (i.e., the angled surfaces 5410 of FIGS. 21A-21B) to bind against the ramped surfaces 5406 (i.e., the angled surfaces 5412 of FIGS. 21A-21B). Consequently, continued rotation of the applicator cap 210 in the second direction B causes the sensor cap 5018 to correspondingly rotate in the same direction and thereby unthread from the mating member 5016 to allow the sensor cap 5018 to detach from the sensor control device 5002. Detaching the sensor cap 5018 from the sensor control device 5002 exposes the distal portions of the sensor 5010 and the sharp 5012, and thus places the sensor control device 5002 in position for firing (use).

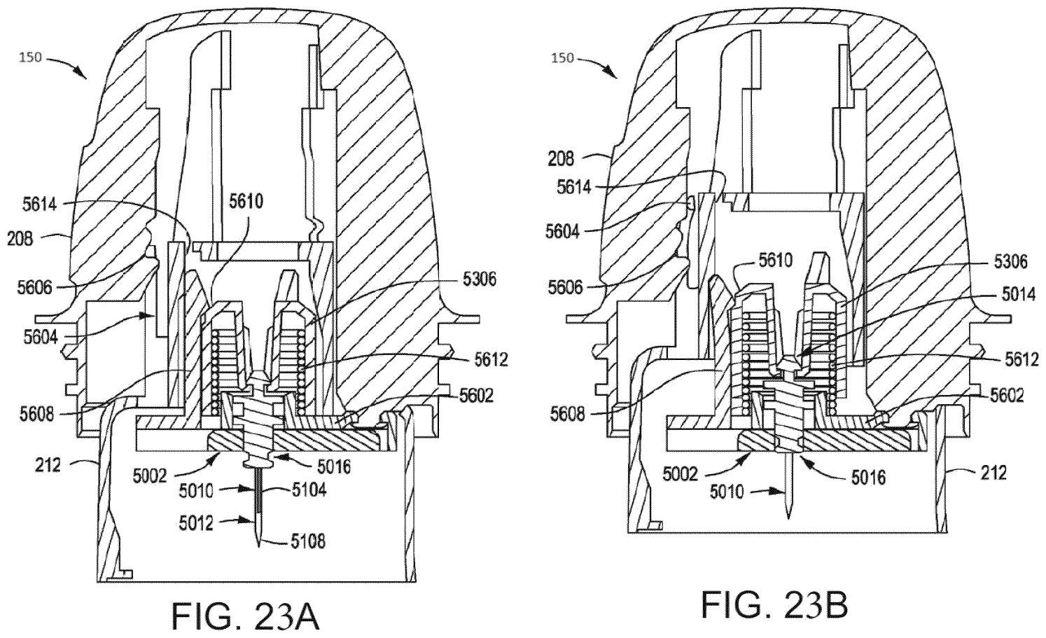

FIGS. 23A and 23B are cross-sectional side views of the applicator 150 ready to deploy the sensor control device 5002 to a target monitoring location, according to one or more embodiments. More specifically, FIG. 23A depicts the applicator 23102 ready to deploy (fire) the sensor control device 5002, and FIG. 23B depicts the sensor applicator 150 in the process of deploying (firing) the sensor control device 5002. As illustrated, the applicator cap 210 (FIGS. 20A-20C and 22) has been removed, which correspondingly detaches (removes) the sensor cap 5018 (FIGS. 20A-20C and 22) and thereby exposes the tail 5104 of the sensor 5010 and the sharp tip 5108 of the sharp 5012, as described above. In conjunction with the sheath 212 and the sharp carrier 5306, the applicator 150 also includes a sensor carrier 5602 (alternately referred to as a “puck” carrier) that helps position and secure the sensor control device 5002 within the applicator 150.

Referring first to FIG. 23A, as illustrated, the sheath 212 includes one or more sheath arms 5604 (one shown) configured to interact with a corresponding one or more detents 5606 (one shown) defined within the interior of housing 208. The detent(s) 5606 are alternately referred to as “firing” detent(s). When the sensor control device 5002 is initially installed in sensor applicator 150, the sheath arms 5604 may be received within the detents 5606, which places the sensor applicator 150 in firing position. In the firing position, the mating member 5016 extends distally beyond the bottom of the sensor control device 5002. As discussed below, the process of firing the sensor applicator 150 causes the mating member 5016 to retract so that it does not contact the user's skin.

The sensor carrier 5602 may also include one or more carrier arms 5608 (one shown) configured to interact with a corresponding one or more grooves 5610 (one shown) defined on the sharp carrier 5306. A spring 5612 may be arranged within a cavity defined by the sharp carrier 5306 and may passively bias the sharp carrier 5306 upward within housing 208. When the carrier arm(s) 5608 are properly received within the groove(s) 5610, however, the sharp carrier 5306 is maintained in position and prevented from moving upward. The carrier arm(s) 5608 interpose the sheath 212 and the sharp carrier 5306, and a radial shoulder 5614 defined on the sheath 212 may be sized to maintain the carrier arm(s) 5608 engaged within the groove(s) 5610 and thereby maintain the sharp carrier 5306 in position.

In FIG. 23B, the sensor applicator 150 is in the process of firing. As discussed herein with reference to FIGS. 3F-3G, this may be accomplished by advancing the sensor applicator 150 toward a target monitoring location until the sheath 212 engages the skin of the user. Continued pressure on the sensor applicator 150 against the skin may cause the sheath arm(s) 5604 to disengage from the corresponding detent(s) 5606, which allows the sheath 212 to collapse into housing 208. As the sheath 212 starts to collapse, the radial shoulder 5614 eventually moves out of radial engagement with the carrier arm(s) 5608, which allows the carrier arm(s) 5608 to disengage from the groove(s) 5610. The passive spring force of the spring 5612 is then free to push upward on the sharp carrier 5306 and thereby force the carrier arm(s) 5608 out of engagement with the groove(s) 5610, which allows the sharp carrier 5306 to move slightly upward within the housing 208. In some embodiments, fewer coils may be incorporated into the design of the spring 5612 to increase the spring force necessary to overcome the engagement between carrier arm(s) 5608 and the groove(s) 5610. In at least one embodiment, one or both of the carrier arm(s) 5608 and the groove(s) 5610 may be angled to help case disengagement.

As the sharp carrier 5306 moves upward within the housing 208, the sharp hub 5014 may correspondingly move in the same direction, which may cause partial retraction of the mating member 5016 such that it becomes flush, substantially flush, or sub-flush with the bottom of the sensor control device 5002. As will be appreciated, this ensures that the mating member 5016 does not come into contact with the user's skin, which might otherwise adversely impact sensor insertion, cause excessive pain, or prevent the adhesive patch (not shown) positioned on the bottom of the sensor control device 5002 from properly adhering to the skin.

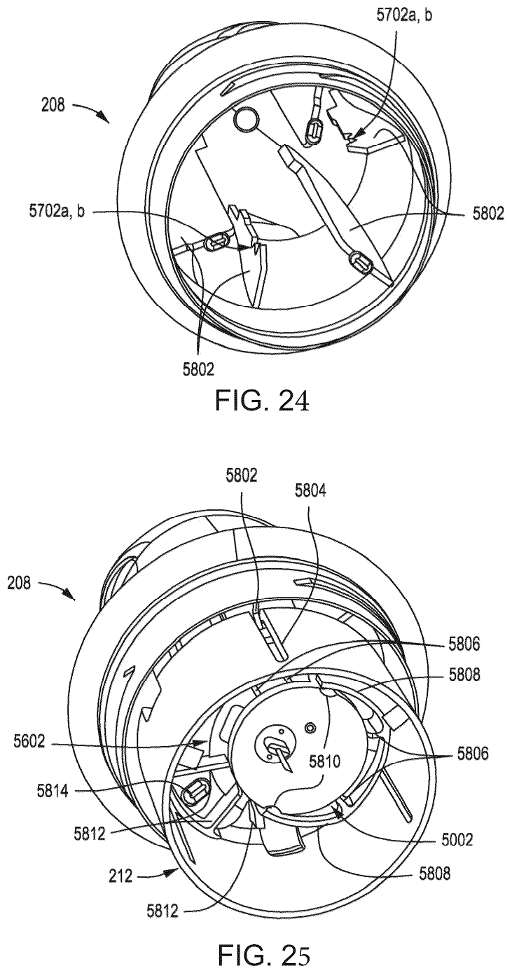

FIG. 24 is an isometric bottom view of housing 208, according to one or more embodiments. As illustrated, one or more longitudinal ribs 5802 (four shown) may be defined within the interior of housing 208. The ribs 5802 may be equidistantly or non-equidistantly spaced from each other and extend substantially parallel to centerline of the housing 208. The first and second detents 5702a, b may be defined on one or more of the longitudinal ribs 5802.

FIG. 25 is an isometric bottom view of housing 208 with the sheath 212 and other components at least partially positioned within housing 208. As illustrated, the sheath 212 may provide or otherwise define one or more longitudinal slots 5804 configured to mate with the longitudinal ribs 5802 of the housing 208. As the sheath 212 collapses into housing 208, as generally described above, the ribs 5802 may be received within the slots 5804 to help maintain the sheath 212 aligned with the housing during its movement. As will be appreciated, this may result in tighter circumferential and radial alignment within the same dimensional and tolerance restrictions of housing 208.

In the illustrated embodiment, the sensor carrier 5602 may be configured to hold the sensor control device 5002 in place both axially (e.g., once the sensor cap 5018 is removed) and circumferentially. To accomplish this, the sensor carrier 5602 may include or otherwise define one or more support ribs 5806 and one or more flexible arms 5808. The support ribs 5806 extend radially inward to provide radial support to the sensor control device 5002. The flexible arms 5808 extend partially about the circumference of the sensor control device 5002 and the ends of the flexible arms 5808 may be received within corresponding grooves 5810 defined in the side of the sensor control device 5002. Accordingly, the flexible arms 5808 may be able to provide both axial and radial support to the sensor control device 5002. In at least one embodiment, the ends of the flexible arms 5808 may be biased into the grooves 5810 of the sensor control device 5002 and otherwise locked in place with corresponding sheath locking ribs 5812 provided by the sheath 212.

In some embodiments, the sensor carrier 5602 may be ultrasonically welded to housing 208 at one or more points 5814. In other embodiments, however, the sensor carrier 5602 may alternatively be coupled to the housing 208 via a snap-fit engagement, without departing from the scope of the disclosure. This may help hold the sensor control device 5002 in place during transport and firing.

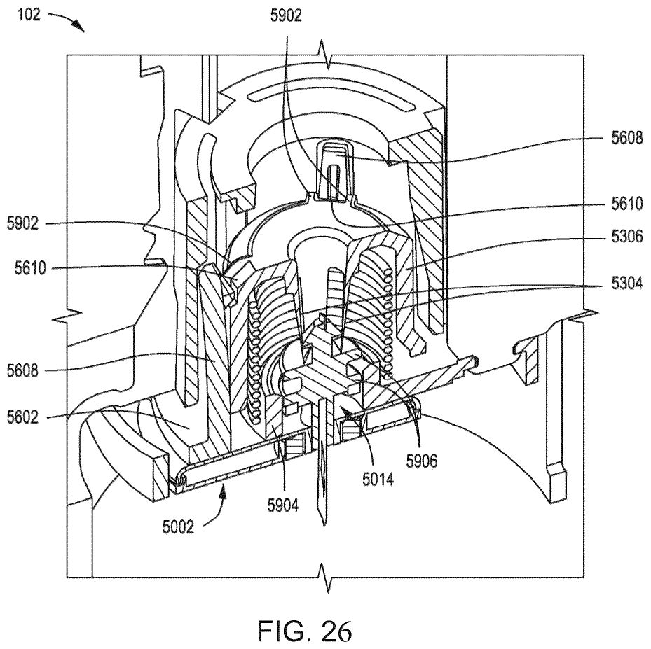

FIG. 26 is an enlarged cross-sectional side view of the sensor applicator 150 with the sensor control device 5002 installed therein, according to one or more embodiments. As discussed above, the sensor carrier 5602 may include one or more carrier arms 5608 (two shown) engageable with the sharp carrier 5306 at corresponding grooves 5610. In at least one embodiment, the grooves 5610 may be defined by pairs of protrusions 5902 defined on the sharp carrier 5306. Receiving carrier arms 5608 within the grooves 5610 may help stabilize the sharp carrier 5306 from unwanted tilting during all stages of retraction (firing).

In the illustrated embodiment, the arms 5304 of the sharp carrier 5306 may be stiff enough to control, with greater refinement, radial and bi-axial motion of the sharp hub 5014. In some embodiments, for example, clearances between the sharp hub 5014 and the arms 5304 may be more restrictive in both axial directions as the relative control of the height of the sharp hub 5014 may be more critical to the design.

In the illustrated embodiment, the sensor carrier 5602 defines or otherwise provides a central boss 5904 sized to receive the sharp hub 5014. In some embodiments, as illustrated, the sharp hub 5014 may provide one or more radial ribs 5906 (two shown). In at least one embodiment, the inner diameter of the central boss 5904 helps provide radial and tilt support to the sharp hub 5014 during the life of sensor applicator 150 and through all phases of operation and assembly. Moreover, having multiple radial ribs 5906 increases the length-to-width ratio of the sharp hub 5014, which also improves support against tilting.

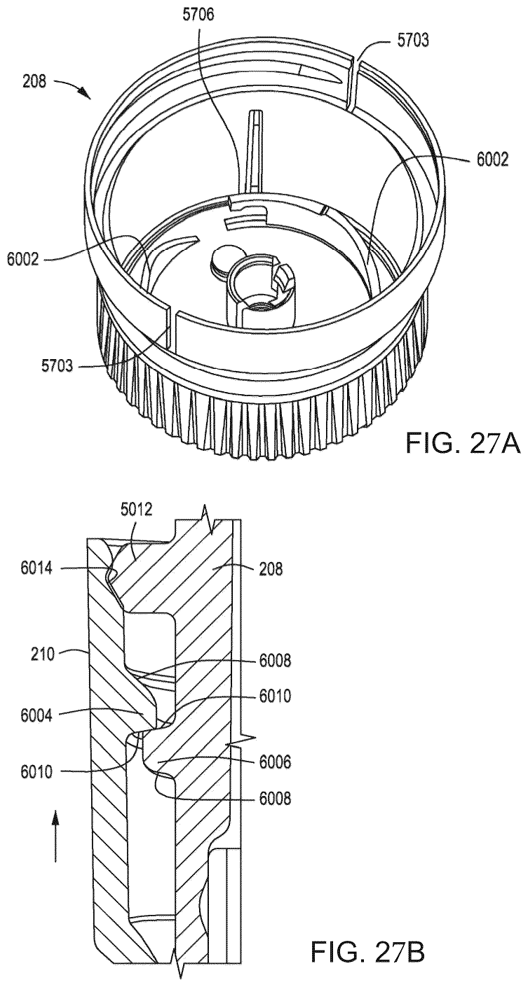

FIG. 27A is an isometric top view of applicator cap 210, according to one or more embodiments. In the illustrated embodiment, two axial slots 5703 are depicted that separate upper portions of the applicator cap 210 near its threading. As mentioned above, slots 5703 may help the applicator cap 210 flex outward to be snapped into engagement with the housing 208. In contrast, the applicator cap 210 may be twisted (unthreaded) off the housing 208 by an end user.

FIG. 27A also depicts the ribs 5706 (one visible) defined by the applicator cap 210. By interlocking with the ribs 5704 defined on sheath 212, the ribs 5706 may help lock the sheath 212 in all directions to prevent premature collapse during a shock or drop event. Sheath 212 may be unlocked when the user unscrews the applicator cap 210 from the housing, as generally described above. As mentioned herein, the top of each rib 5706 may provide a corresponding ramped surface 6002, and as the applicator cap 210 is rotated to unthread from the housing 208, the ribs 5704 defined on the sheath 212 may slidingly engage the ramped surfaces 6002, which results in the upward displacement of the sheath 212 into the housing 208.

In some embodiments, additional features may be provided within the interior of the applicator cap 210 to hold a desiccant component that maintains proper moisture levels through shelf life. Such additional features may be snaps, posts for press-fitting, heat-staking, ultrasonic welding, etc.

FIG. 27B is an enlarged cross-sectional view of the engagement between the applicator cap 210 and the housing 208, according to one or more embodiments. As illustrated, applicator cap 210 may define a set of inner threads 6004 and housing 208 may define a set of outer threads 6006 engageable with the inner threads 6004. As mentioned herein, the applicator cap 210 may be snapped onto the housing 208, which may be accomplished by advancing the inner threads 6004 axially past the outer threads 6006 in the direction indicated by the arrow, which causes the applicator cap 210 to flex outward. To help case this transition, as illustrated, corresponding surfaces 6008 of the inner and outer threads 6004, 6006 may be curved, angled, or chamfered. Corresponding flat surfaces 6010 may be provided on each thread 6004, 6006 and configured to matingly engage once the applicator cap 210 is properly snapped into place on the housing 208. The flat surfaces 6010 may slidingly engage one another as the user unthreads the applicator cap 210 from housing 208.

The threaded engagement between the applicator cap 210 and the housing 208 results in a sealed engagement that protects the inner components against moisture, dust, etc. In some embodiments, housing 208 may define or otherwise provide a stabilizing feature 6012 configured to be received within a corresponding groove 1914 defined on the applicator cap 210. The stabilizing feature 6012 may help stabilize and stiffen the applicator cap 210 once the applicator cap 210 is snapped onto housing 208. This may prove advantageous in providing additional drop robustness to the sensor applicator 150. This may also help increase the removal torque of the applicator cap 210.

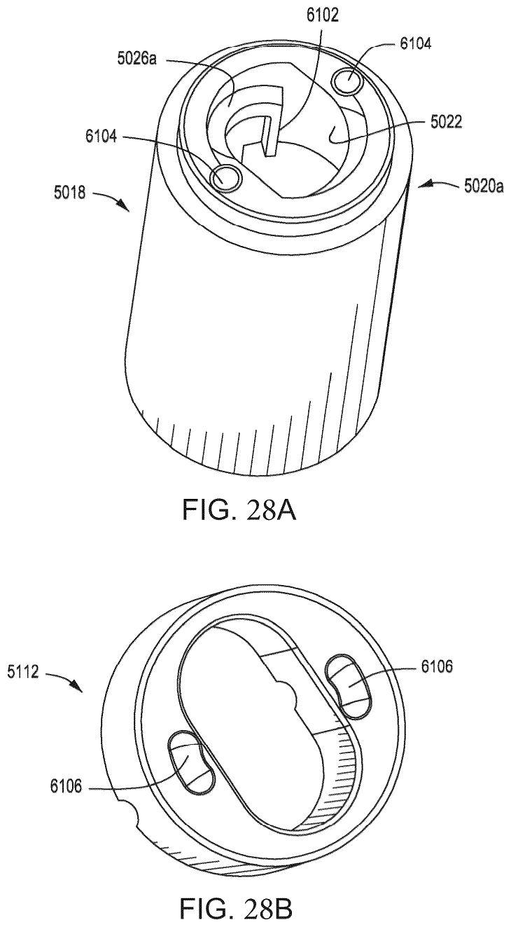

FIGS. 28A and 28B are isometric views of the sensor cap 5018 and the collar 5112, respectively, according to one or more embodiments. Referring to FIG. 28A, in some embodiments, the sensor cap 5018 may comprise an injection molded part. This may prove advantageous in molding the internal threads 5026a defined within the inner chamber 5022, as opposed to installing a threaded core or threading the inner chamber 5022. In some embodiments, one or more stop ribs 6102 (on visible) may be defined within the inner chamber 5022 to prevent over travel relative to mating member 5016 of the sharp hub 5014 (FIGS. 16A-16B).

Referring to both FIGS. 28A and 28B, in some embodiments, one or more protrusions 6104 (two shown) may be defined on the first end 5020a of the sensor cap 5018 and configured to mate with one or more corresponding indentations 6106 (two shown) defined on the collar 5112. In other embodiments, however, the protrusions 6104 may instead be defined on the collar 5112 and the indentations 6106 may be defined on the sensor cap 5018, without departing from the scope of the disclosure.

The mateable protrusions 6104 and indentations 6106 may prove advantageous in rotationally locking the sensor cap 5018 to prevent unintended unscrewing of the sensor cap 5018 from the collar 5112 (and thus the sensor control device 5002) during the life of the sensor applicator 150 and through all phases of operation/assembly. In some embodiments, as illustrated, the indentations 6106 may be formed or otherwise defined in the general shape of a kidney bean. This may prove advantageous in allowing for some over-rotation of the sensor cap 5018 relative to the collar 5112. Alternatively, the same benefit may be achieved via a flat end threaded engagement between the two parts.

### Example Embodiments of Seal Arrangement for Analyte Monitoring Systems

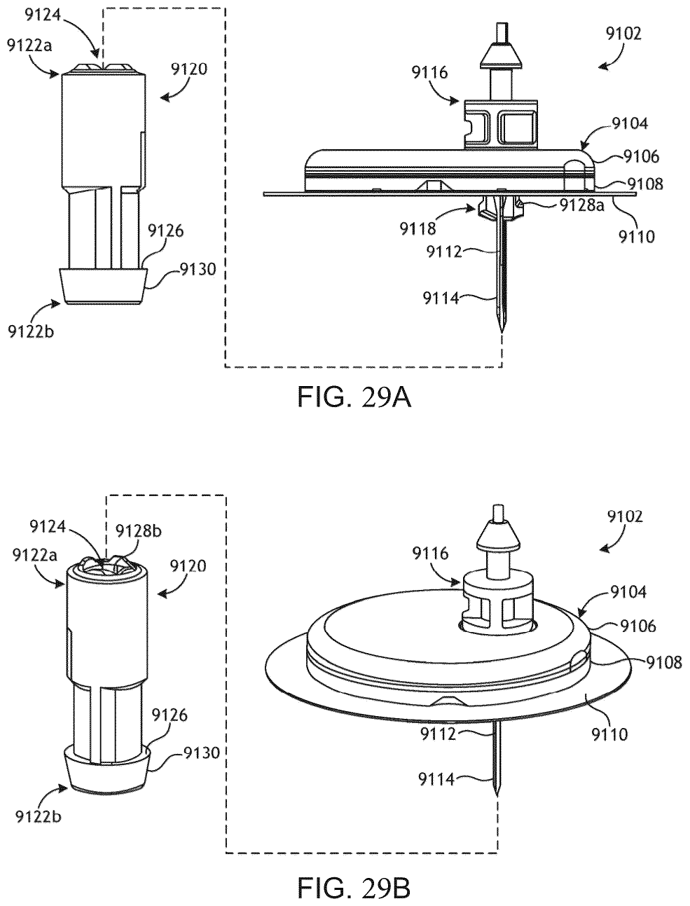

FIGS. 29A and 29B are side and isometric views, respectively, of an example sensor control device 9102, according to one or more embodiments of the present disclosure. The sensor control device 9102 may be similar in some respects to the sensor control device 102 of FIG. 1 and therefore may be best understood with reference thereto. Moreover, the sensor control device 9102 may replace the sensor control device 102 of FIG. 1 and, therefore, may be used in conjunction with the sensor applicator 150 of FIG. 1, which may deliver the sensor control device 9102 to a target monitoring location on a user's skin.

As illustrated, the sensor control device 9102 includes an electronics housing 9104, which may be generally disc-shaped and have a circular cross-section. In other embodiments, however, the electronics housing 9104 may exhibit other cross-sectional shapes, such as ovoid, oval, or polygonal, without departing from the scope of the disclosure. The electronics housing 9104 includes a shell 9106 and a mount 9108 that is mateable with the shell 9106. The shell 9106 may be secured to the mount 9108 via a variety of ways, such as a snap fit engagement, an interference fit, sonic welding, laser welding, one or more mechanical fasteners (e.g., screws), a gasket, an adhesive, or any combination thereof. In some cases, shell 9106 may be secured to the mount 9108 such that a sealed interface is generated therebetween. An adhesive patch 9110 may be positioned on and otherwise attached to the underside of the mount 9108. Similar to the adhesive patch 108 of FIG. 1, the 9110 may be configured to secure and maintain the sensor control device 9102 in position on the user's skin during operation.

The sensor control device 9102 may further include a sensor 9112 and a sharp 9114 used to help deliver the sensor 9112 transcutaneously under a user's skin during application of the sensor control device 9102. Corresponding portions of the sensor 9112 and the sharp 9114 extend distally from the bottom of the electronics housing 9104 (e.g., the mount 9108). A sharp hub 9116 may be overmolded onto the sharp 9114 and configured to secure and carry the sharp 9114. As best seen in FIG. 29A, the sharp hub 9116 may include or otherwise define a mating member 9118. In assembling the sharp 9114 to the sensor control device 9102, the sharp 9114 may be advanced axially through the electronics housing 9104 until the sharp hub 9116 engages an upper surface of the electronics housing 9104 or an internal component thereof and the mating member 9118 extends distally from the bottom of the mount 9108. As described herein below, in at least one embodiment, the sharp hub 9116 may sealingly engage an upper portion of a seal overmolded onto the mount 9108. As the sharp 9114 penetrates the electronics housing 9104, the exposed portion of the sensor 9112 may be received within a hollow or recessed (arcuate) portion of the sharp 9114. The remaining portion of the sensor 9112 is arranged within the interior of the electronics housing 9104.

The sensor control device 9102 may further include a sensor cap 9120, shown detached from the electronics housing 9104 in FIGS. 29A-29B. The sensor cap 9120 may help provide a sealed barrier that surrounds and protects exposed portions of the sensor 9112 and the sharp 9114. As illustrated, the sensor cap 9120 may comprise a generally cylindrical body having a first end 9122a and a second end 9122b opposite the first end 9122a. The first end 9122a may be open to provide access into an inner chamber 9124 defined within the body. In contrast, the second end 9122b may be closed and may provide or otherwise define an engagement feature 9126. As described in more detail below, the engagement feature 9126 may help mate the sensor cap 9120 to an applicator cap of a sensor applicator (e.g., the sensor applicator 150 of FIG. 1), and may help remove the sensor cap 9120 from the sensor control device 9102 upon removing the sensor cap from the sensor applicator.

The sensor cap 9120 may be removably coupled to the electronics housing 9104 at or near the bottom of mount 9108. More specifically, the sensor cap 9120 may be removably coupled to the mating member 9118, which extends distally from the bottom of mount 9108. In at least one embodiment, for example, the mating member 9118 may define a set of external threads 9128a (FIG. 29A) mateable with a set of internal threads 9128b (FIG. 29B) defined within the inner chamber 9124 of the sensor cap 9120. In some embodiments, the external and internal threads 9128a, b may comprise a flat thread design (e.g., lack of helical curvature), but may alternatively comprise a helical threaded engagement. Accordingly, in at least one embodiment, the sensor cap 9120 may be threadably coupled to the sensor control device 9102 at the mating member 9118 of the sharp hub 9116. In other embodiments, the sensor cap 9120 may be removably coupled to the mating member 9118 via other types of engagements including, but not limited to, an interference or friction fit, or a frangible member or substance (e.g., wax, an adhesive, etc.) that may be broken with minimal separation force (e.g., axial or rotational force).

In some embodiments, the sensor cap 9120 may comprise a monolithic (singular) structure extending between the first and second ends 9122a, b. In other embodiments, however, the sensor cap 9120 may comprise two or more component parts. In the illustrated embodiment, for example, the body of the sensor cap 9120 may include a desiccant cap 9130 arranged at the second end 9122b. The desiccant cap 9130 may house or comprise a desiccant to help maintain preferred humidity levels within the inner chamber 9124. Moreover, the desiccant cap 9130 may also define or otherwise provide the engagement feature 9126 of the sensor cap 9120. In at least one embodiment, the desiccant cap 9130 may comprise an elastomeric plug inserted into the bottom end of the sensor cap 9120.

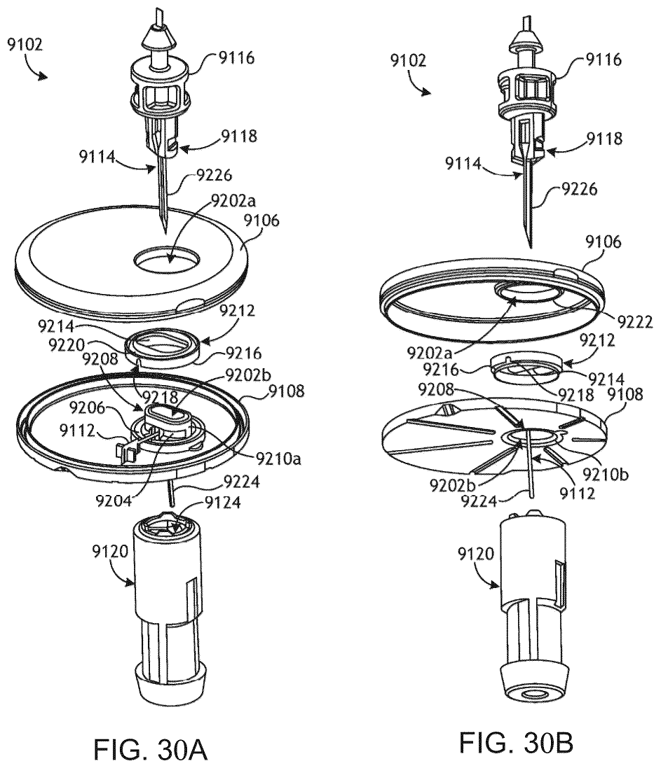

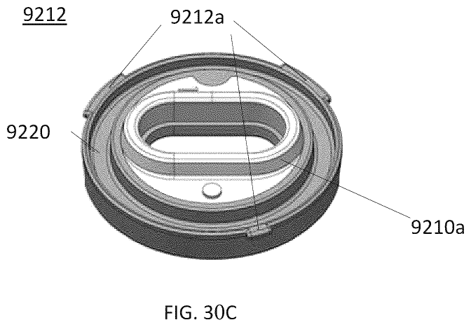

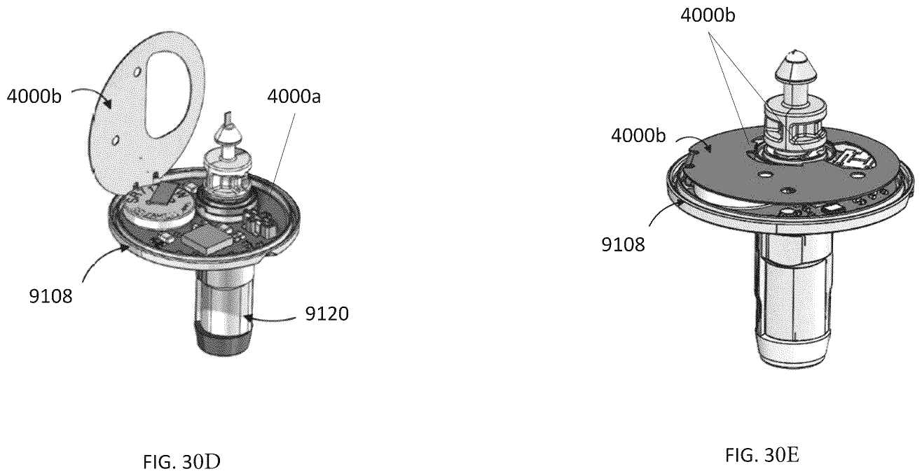

FIGS. 30A and 30B are exploded, isometric top and bottom views, respectively, of the sensor control device 9102, according to one or more embodiments. The shell 9106 and the mount 9108 operate as opposing clamshell halves that enclose or otherwise substantially encapsulate various electronic components (not shown) of the sensor control device 9102. Example electronic components that may be arranged between the shell 9106 and the mount 9108 include, but are not limited to, a battery, resistors, transistors, capacitors, inductors, diodes, and switches.

The shell 9106 may define a first aperture 9202a and the mount 9108 may define a second aperture 9202b, and the apertures 9202a, b may align when the shell 9106 is properly mounted to the mount 9108. As best seen in FIG. 30A, mount 9108 may provide or otherwise define a pedestal 9204 that protrudes from the inner surface of the mount 9108 at the second aperture 9202b. The pedestal 9204 may define at least a portion of the second aperture 9202b. Moreover, channel 9206 may be defined on the inner surface of mount 9108 and may circumscribe pedestal 9202. In the illustrated embodiment, channel 9206 is circular in shape, but could alternatively be another shape, such as oval, ovoid, or polygonal.

Mount 9108 may comprise a molded part made of a rigid material, such as plastic or metal. In some embodiments, a seal 9208 may be overmolded onto mount 9108 and may be made of an elastomer, rubber, a-polymer, or another pliable material suitable for facilitating a sealed interface. In embodiments where the mount 9108 is made of a plastic, the mount 9108 may be molded in a first “shot” of injection molding, and the seal 9208 may be overmolded onto the mount 9108 in a second “shot” of injection molding. Accordingly, the mount 9108 may be referred to or otherwise characterized as a “two-shot mount.”

In the illustrated embodiment, the seal 9208 may be overmolded onto the mount 9108 at the pedestal 9204 and also on the bottom of the mount 9108. More specifically, the seal 9208 may define or otherwise provide a first seal element 9210a overmolded onto the pedestal 9204, and a second seal element 9210b (FIG. 30B) interconnected to (with) the first seal element 9210a and overmolded onto the mount 9108 at the bottom of the mount 9108. In some embodiments, one or both of the seal elements 9210a, b may help form corresponding portions (sections) of the second aperture 9202b. While the seal 9208 is described herein as being overmolded onto the mount 9108, it is also contemplated herein that one or both of the seal elements 9210a, b may comprise an elastomeric component part independent of the mount 9208, such as an O-ring or a gasket.

The sensor control device 9102 may further include a collar 9212 disposed between shell 9106 and mount 9208 and may be a generally annular structure that defines a central aperture 9214. The central aperture 9214 may be sized to receive the first seal element 9210a and may align with both the first and second apertures 9202a, b when the sensor control device 9102 is properly assembled. The shape of the central aperture 9214 may generally match the shape of the second aperture 9202b and the first seal element 9210a.

In some embodiments, the collar 9212 may define or otherwise provide an annular lip 9216 on its bottom surface. The annular lip 9216 may be sized and otherwise configured to mate with or be received into the channel 9206 defined on the inner surface of mount 9108. In some embodiments, a groove 9218 may be defined on the annular lip 9216 and may be configured to accommodate or otherwise receive a portion of the sensor 9112 extending laterally within mount 9108. In some embodiments, the collar 9212 may further define or otherwise provide a collar channel 9220 (FIG. 30A) on its upper surface sized to receive and otherwise mate with an annular ridge 9222 (FIG. 30B) defined on the inner surface of the shell 9106 when the sensor control device 9102 is properly assembled. In some embodiments, collar 9212 may further include a plurality of tabs 9212a (FIG. 30C) on its outer periphery. Tabs 9212a may be located on the same plane as the top surface of collar 9212 and create a shelf for a circuit board. In some embodiments, the circuit board can be foldable and/or a flexible PCB, as described in U.S. Provisional Application No. 63/081,223, filed Sep. 21, 2020, which is incorporated herein by reference in its entirety. For example, PCB 4000 can be mounted within the electronics housing on the plurality of tabs 9212a. As can be seen in FIG. 30D, after PCB 4000 has been loaded onto the sensor mount 9108, first portion 4000a of PCB may be located under tabs 9212a (not shown in FIG. 30D) and rest on the sensor mount 9108. Thereafter, second portion 4000b of PCB 4000 may be folded over first portion 4000a and mounted on the plurality of tabs 9212a. In some embodiments, tabs 9212a can be of the same size or of different sizes and may be equally spaced along the collar 9212's outer periphery. In some embodiments, tabs 9212a can extend from the top surface of the collar 9212, the bottom surface of the collar 9212, or an intermediate location of the collar 9212 therebetween. As shown in FIG. 30C, collar 9212 can include three tabs 9212a and tabs 9212a can be of different sizes, for example, with two tabs being the same size as each other and longer (i.e., as measured along the outer periphery of collar 9212) than a third tab. Additionally, tabs 9212a can be the same or different widths (i.e., measured as distance extending radially outwards). In some embodiments, as can be seen in FIG. 30E, PCB 4000 can include one or more tabs 4000c corresponding to tabs 9212a and mated with and fixed to tabs 9212a.

The sensor 9112 may include a tail 9224 that extends through the second aperture 9202b defined in the mount 9108 to be transcutaneously received beneath a user's skin. The tail 9224 may have an enzyme or other chemistry included thereon to help facilitate analyte monitoring. The sharp 9114 may include a sharp tip 9226 extendable through the first aperture 9202a defined by the shell 9106. As the sharp tip 9226 penetrates the electronics housing 9104, the tail 9224 of the sensor 9112 may be received within a hollow or recessed portion of the sharp tip 9226. The sharp tip 9226 may be configured to penetrate the skin while carrying the tail 9224 to put the active chemistry of the tail 9224 into contact with bodily fluids.

The sensor control device 9102 may provide a sealed subassembly that includes, among other component parts, portions of the shell 9106, the sensor 9112, the sharp 9114, the seal 9208, the collar 9212, and the sensor cap 9120. The sealed subassembly may help isolate the sensor 9112 and the sharp 9114 within the inner chamber 9124 (FIG. 30A) of the sensor cap 9120. In assembling the sealed subassembly, the sharp tip 9226 is advanced through the electronics housing 9104 until the sharp hub 9116 engages the seal 9208 and, more particularly, the first seal element 9210a. The mating member 9118 provided at the bottom of the sharp hub 9116 may extend out the second aperture 9202b in the bottom of the mount 9108, and the sensor cap 9120 may be coupled to the sharp hub 9116 at the mating member 9118. Coupling the sensor cap 9120 to the sharp hub 9116 at the mating member 9118 may urge the first end 9122a of the sensor cap 9120 into sealed engagement with the seal 9208 and, more particularly, into sealed engagement with the second seal element 9210b on the bottom of the mount 9108. In some embodiments, as the sensor cap 9120 is coupled to the sharp hub 9116, a portion of the first end 9122a of the sensor cap 9120 may bottom out (engage) against the bottom of the mount 9108, and the sealed engagement between the sensor hub 9116 and the first seal element 9210a may be able to assume any tolerance variation between features.

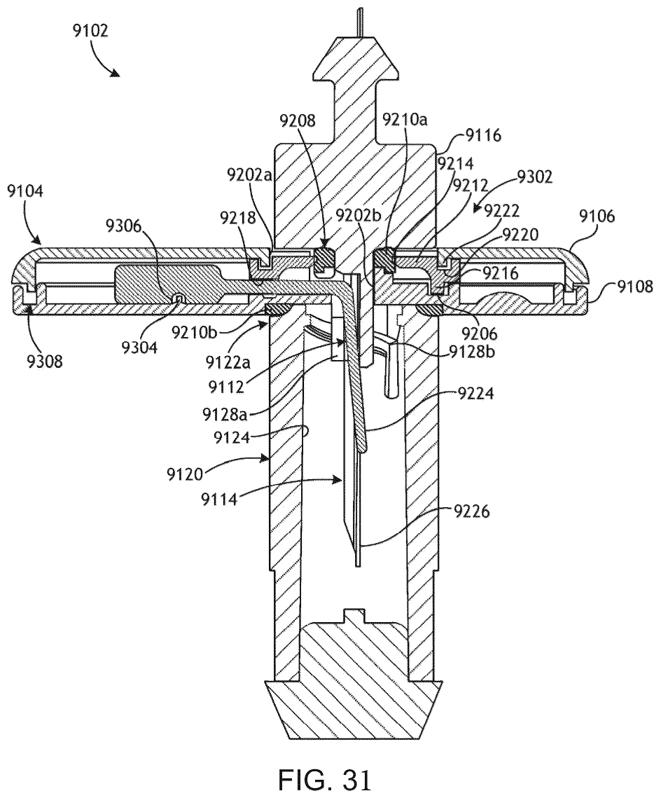

FIG. 31 is a cross-sectional side view of sensor control device 9102, according to one or more embodiments. As indicated above, the sensor control device 9102 may include or otherwise incorporate a sealed subassembly 9302, which may be useful in isolating the sensor 9112 and the sharp 9114 within the inner chamber 9124 of the sensor cap 9120. To assemble the sealed subassembly 9302, the sensor 9112 may be located within mount 9108 such that the tail 9224 extends through the second aperture 9202b at the bottom of the mount 9108. In at least one embodiment, a locating feature 9304 may be defined on the inner surface of the mount 9108, and the sensor 9112 may define a groove 9306 that is mateable with the locating feature 9304 to properly locate the sensor 9112 within the mount 9108.

Once the sensor 9112 is properly located, the collar 9212 may be installed on mount 9108. More specifically, the collar 9212 may be positioned such that the first seal element 9210a of the seal 9208 is received within the central aperture 9214 defined by the collar 9212 and the first seal element 9210a generates a radial seal against the collar 9212 at the central aperture 9214. Moreover, the annular lip 9216 defined on the collar 9212 may be received within the channel 9206 defined on the mount 9108, and the groove 9218 defined through the annular lip 9216 may be aligned to receive the portion of the sensor 9112 that traverses the channel 9206 laterally within the mount 9108. In some embodiments, an adhesive may be injected into channel 9206 to secure the collar 9212 to the mount 9108. The adhesive may also facilitate a sealed interface between the two components and generate a seal around the sensor 9112 at groove 9218, which may isolate the tail 9224 from the interior of the electronics housing 9104.

The shell 9106 may then be mated with or otherwise coupled to the mount 9108. In some embodiments, as illustrated, the shell 9106 may mate with the mount 9108 via a tongue-and-groove engagement 9308 at the outer periphery of the electronics housing 9104. An adhesive may be injected (applied) into the groove portion of the engagement 9308 to secure the shell 9106 to the mount 9108, and also to create a sealed engagement interface. Mating the shell 9106 to the mount 9108 may also cause the annular ridge 9222 defined on the inner surface of the shell 9106 to be received within the collar channel 9220 defined on the upper surface of the collar 9212. In some embodiments, an adhesive may be injected into the collar channel 9220 to secure the shell 9106 to the collar 9212, and also to facilitate a sealed interface between the two components at that location. When shell 9106 mates with mount 9108, the first seal element 9210a may extend at least partially through (into) the first aperture 9202a defined in the shell 9106.

The sharp 9114 may then be coupled to the sensor control device 9102 by extending the sharp tip 9226 through the aligned first and second apertures 9202a, b defined in the shell 9106 and the mount 9108, respectively. The sharp 9114 may be advanced until the sharp hub 9116 engages the seal 9208 and, more particularly, engages the first seal element 9210a. The mating member 9118 may extend (protrude) out the second aperture 9202b at the bottom of the mount 9108 when the sharp hub 9116 engages the first seal element 9210a.

The sensor cap 9120 may then be removably coupled to the sensor control device 9102 by threadably mating the internal threads 9128b of the sensor cap 9120 with the external threads 9128a of the mating member 9118. The inner chamber 9124 may be sized and otherwise configured to receive the tail 9224 and the sharp tip 9226 extending from the bottom of the mount 9108. Moreover, the inner chamber 9124 may be sealed to isolate the tail 9224 and the sharp tip 9226 from substances that might adversely interact with the chemistry of the tail 9224. In some embodiments, a desiccant (not shown) may be present within the inner chamber 9124 to maintain proper humidity levels.

Tightening (rotating) the mated engagement between the sensor cap 9120 and the mating member 9118 may urge the first end 9122a of the sensor cap 9120 into sealed engagement with the second seal element 9210b in an axial direction (e.g., along the centerline of the apertures 9202a, b), and may further enhance the sealed interface between the sharp hub 9116 and the first seal element 9210a in the axial direction. Moreover, tightening the mated engagement between the sensor cap 9120 and the mating member 9118 may compress the first seal element 9210a, which may result in an enhanced radial sealed engagement between the first seal element 9210a and the collar 9212 at the central aperture 9214. Accordingly, in at least one embodiment, the first seal element 9210a may help facilitate axial and radial sealed engagements.

As mentioned above, the first and second seal elements 9210a, b may be overmolded onto the mount 9108 and may be physically linked or otherwise interconnected. Consequently, a single injection molding shot may flow through the second aperture 9202b of the mount 9108 to create both ends of the seal 9208. This may prove advantageous in being able to generate multiple sealed interfaces with only a single injection molded shot. An additional advantage of a two-shot molded design, as opposed to using separate elastomeric components (e.g., O-rings, gaskets, etc.), is that the interface between the first and second shots is a reliable bond rather than a mechanical seal. Hence, the effective number of mechanical sealing barriers is effectively cut in half. Moreover, a two-shot component with a single elastomeric shot also has implications to minimizing the number of two-shot components needed to achieve all the necessary sterile barriers. Once properly assembled, the sealed subassembly 9302 may be subjected to a radiation sterilization process to sterilize the sensor 9112 and the sharp 9114. The sealed subassembly 9302 may be subjected to radiation sterilization prior to or after coupling the sensor cap 9120 to the sharp hub 9116. When sterilized after coupling the sensor cap 9120 to the sharp hub 9116, the sensor cap 9120 may be made of a material that permits the propagation of radiation therethrough. In some embodiments, the sensor cap 9120 may be transparent or translucent, but can otherwise be opaque, without departing from the scope of the disclosure.

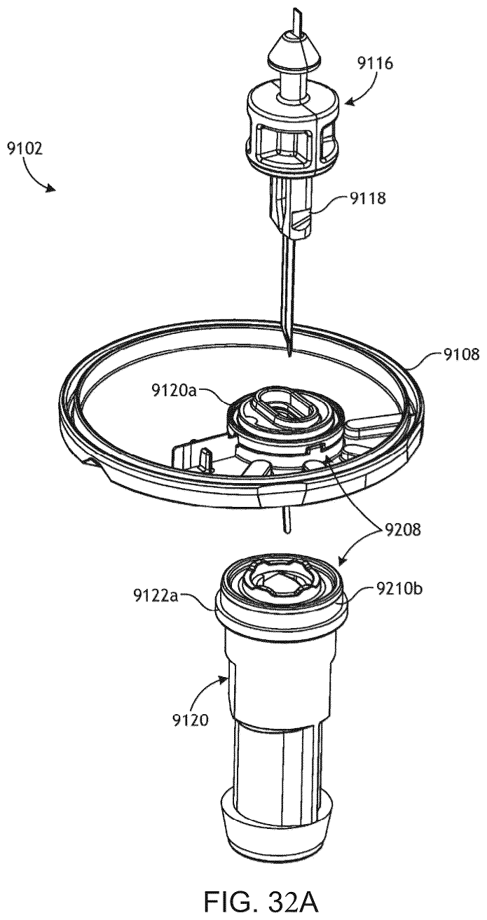

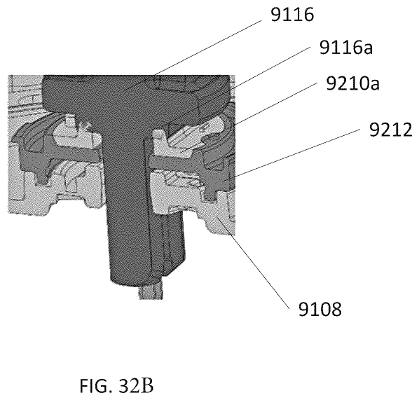

FIG. 32A is an exploded isometric view of a portion of another embodiment of the sensor control device 9102 of FIGS. 30A-30B and 31A-31B. Embodiments included above describe the mount 9108 and the seal 9208 being manufactured via a two-shot injection molding process. In other embodiments, however, as briefly mentioned above, one or both of the seal elements 9210a, b of the seal 9208 may comprise an elastomeric component part independent of mount 9208. In the illustrated embodiment, for example, the first seal element 9210a may be overmolded onto the collar 9212 and the second seal element 9210b may be overmolded onto the sensor cap 9120. Alternatively, the first and second seal elements 9210a, b may comprise a separate component part, such as a gasket or O-ring 9210a positioned on the upper surface of the collar 9212 (FIGS. 30C and 32A) and the sensor cap 9120, respectively. Tightening (rotating) the mated engagement between the sensor cap 9120 and the mating member 9118 may urge the second seal element 9210b into sealed engagement with the bottom of the mount 9108 in an axial direction and may enhance a sealed interface between the sharp hub 9116 and the first seal element 9210a in the axial direction. In some embodiments, as shown in FIG. 32B, sharp hub 9116 may include a raised mating surface 9116a (e.g., a ledge) configured to mate with seal element 9210a. As such, tightening (rotating) the mated engagement between the sensor cap 9120 and the mating member 9118 may enhance a sealed interface between the mating surface 9116a and the first seal element 9210a in the axial direction.

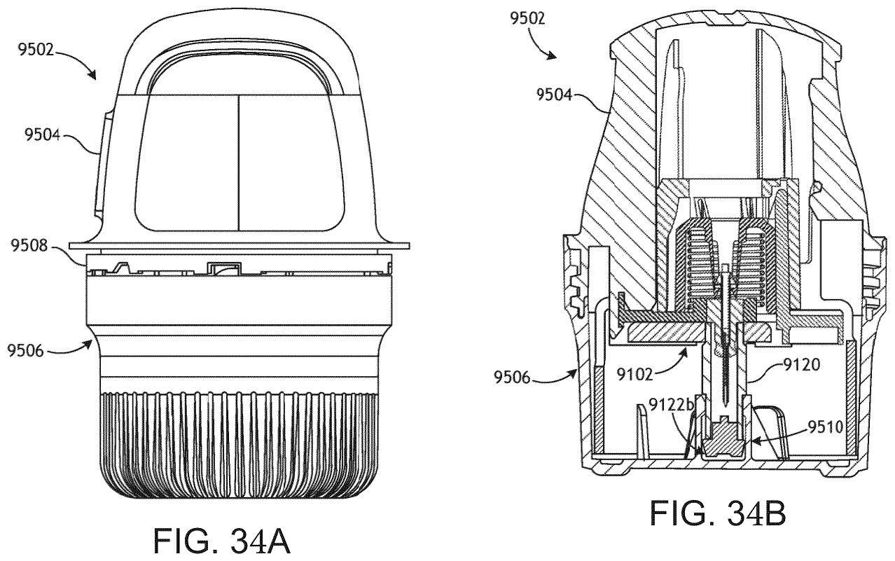

FIG. 33A is an isometric bottom view of mount 9108, and FIG. 34B is an isometric top view of the sensor cap 9120, according to one or more embodiments. As shown in FIG. 33A, the mount 9108 may provide or otherwise define one or more indentations or pockets 9402 at or near the opening to the second aperture 9202b. As shown in FIG. 33B, the sensor cap 9120 may provide or otherwise define one or more projections 9404 at or near the first end 9122a of the sensor cap 9120. The projections 9404 may be received within pockets 9402 when the sensor cap 9120 is coupled to the sharp hub 9116 (FIGS. 30A-30B and 31). More specifically, as described above, as the sensor cap 9120 is coupled to the mating member 9118 (FIGS. 31A-31B and 93) of the sensor hub 9116, the first end 9122a of the sensor cap 9120 is brought into sealed engagement with the second seal element 9210b. In this process, the projections 9404 may also be received within the pockets 9402, which may help prevent premature unthreading of the sensor cap 9120 from the sharp hub 9116.

FIGS. 34A and 34B are side and cross-sectional side views, respectively, of an example sensor applicator 9502, according to one or more embodiments. The sensor applicator 9502 may be similar in some respects to the sensor applicator 150 of FIG. 1 and, therefore, may be designed to deliver (fire) a sensor control device, such as the sensor control device 9102. FIG. 34A depicts how the sensor applicator 9502 might be shipped to and received by a user, and FIG. 34B depicts the sensor control device 9102 arranged within the interior of the sensor applicator 9502.

As shown in FIG. 34A, the sensor applicator 9502 includes a housing 9504 and an applicator cap 9506 removably coupled to the housing 9504. In some embodiments, the applicator cap 9506 may be threaded to the housing 9504 and include a tamper ring 9508. Upon rotating (e.g., unscrewing) the applicator cap 9506 relative to the housing 9504, the tamper ring 9508 may shear and thereby free the applicator cap 9506 from the sensor applicator 9502.

In FIG. 34B, the sensor control device 9102 is positioned within sensor applicator 9502. Once the sensor control device 9102 is fully assembled, it may then be loaded into the sensor applicator 9502 and the applicator cap 9506 may be coupled to the sensor applicator 9502. In some embodiments, the applicator cap 9506 and the housing 9504 may have opposing, mateable sets of threads that enable the applicator cap 9506 to be screwed onto the housing 9504 in a clockwise (or counterclockwise) direction and thereby secure the applicator cap 9506 to the sensor applicator 9502.

Securing the applicator cap 9506 to the housing 9504 may also cause the second end 9122b of the sensor cap 9120 to be received within a cap post 9510 located within the interior of the applicator cap 9506 and extending proximally from the bottom thereof. The cap post 9510 may be configured to receive at least a portion of the sensor cap 9120 as the applicator cap 9506 is coupled to the housing 9504.

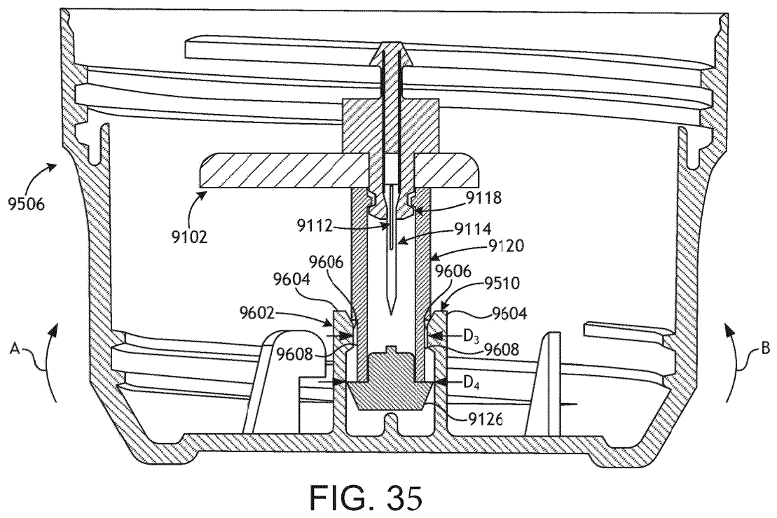

FIG. 35 is a cross-sectional side view of the sensor control device 9102 positioned within the applicator cap 9506, according to one or more embodiments. As illustrated, the opening to the receiver feature 9602 exhibits a first diameter D3, while the engagement feature 9126 of the sensor cap 9120 exhibits a second diameter D4 that is larger than the first diameter D3 and greater than the outer diameter of the remaining portions of the sensor cap 9120. As the sensor cap 9120 is extended into the cap post 9510, the compliant member(s) 9604 of the receiver feature 9602 may flex (expand) radially outward to receive the engagement feature 9126. In some embodiments, as illustrated, the engagement feature 9126 may provide or otherwise define an angled outer surface that helps bias the compliant member(s) 9604 radially outward. Once the engagement feature 9126 bypasses the receiver feature 9602, the compliant member(s) 9604 are able to flex back to (or towards) their natural state and thus lock the sensor cap 9120 within the cap post 9510.

As the applicator cap 9506 is threaded to (screwed onto) the housing 9504 (FIGS. 34A-34B) in the first direction A, the cap post 9510 correspondingly rotates in the same direction and the sensor cap 9120 is progressively introduced into the cap post 9510. As the cap post 9510 rotates, the ramped surfaces 9606 of the compliant members 9604 ratchet against the opposing camming surfaces 9608 of the sensor cap 9120. This continues until the applicator cap 9506 is fully threaded onto (screwed onto) the housing 9504. In some embodiments, the ratcheting action may occur over two full revolutions of the applicator cap 9506 before the applicator cap 9506 reaches its final position.

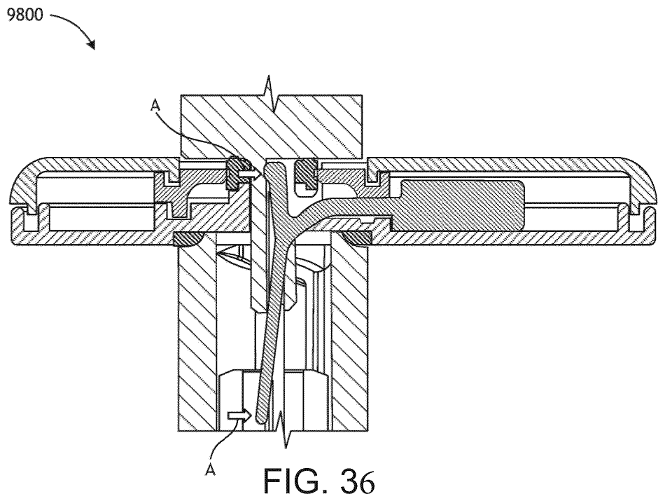

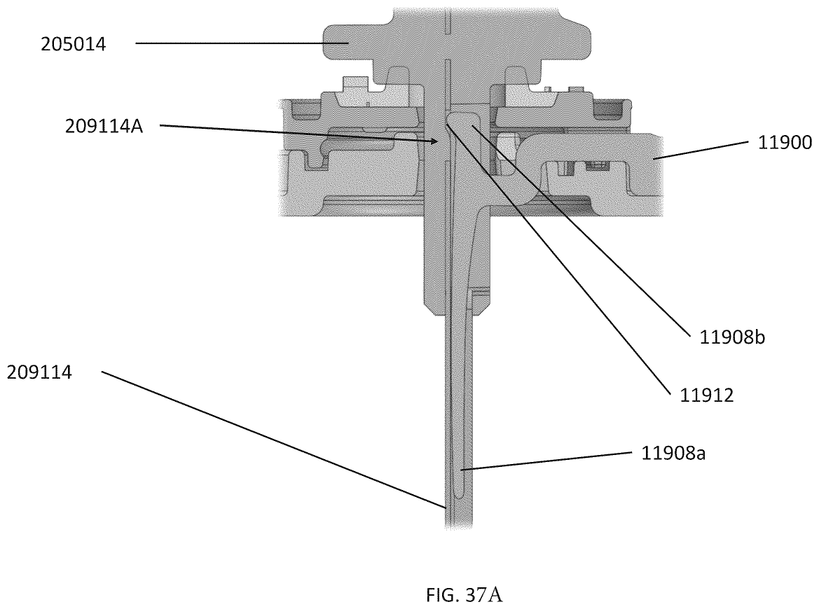

FIG. 36 is a cross-sectional view of a sensor control device 9800 showing example interaction between the sensor and the sharp. After assembly of the sharp, the sensor should sit in a channel defined by the sharp. The sensor control device in FIG. 9 does not show the sensor deflected inwards and otherwise aligned fully with the sharp, but such may be the case upon full assembly as slight bias forces may be assumed by the sensor at the locations indicated by the two arrows A. Biasing the sensor against the sharp may be advantageous so that any relative motion between the sensor and the sharp during subcutaneous insertion does not expose the sensor tip (i.e., the tail) outside the sharp channel, which could potentially cause an insertion failure.

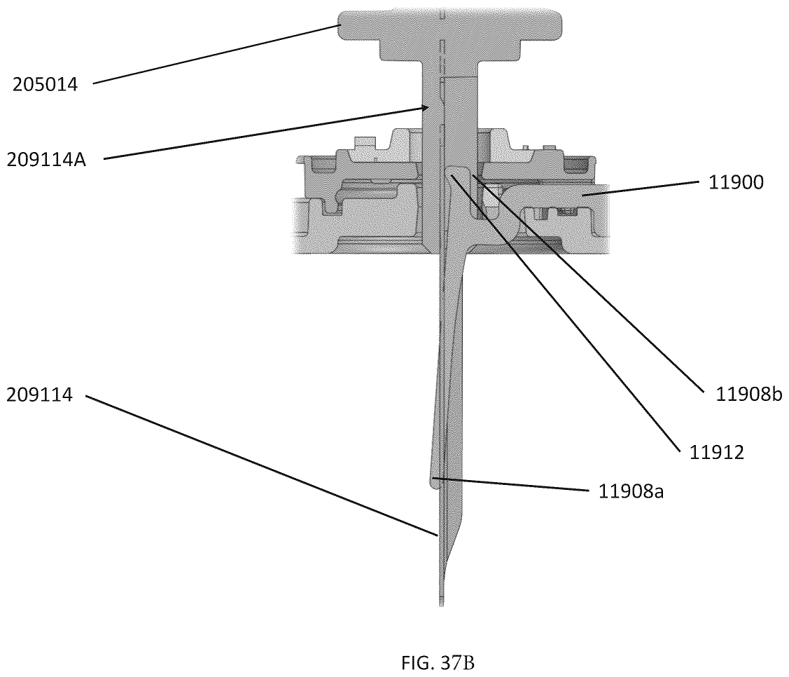

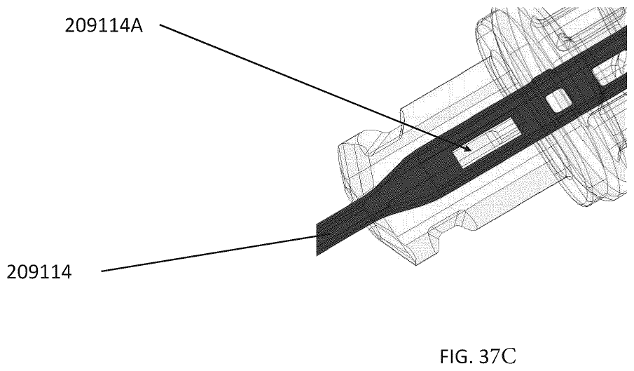

FIGS. 37A-37C illustrates an example embodiment of a sharp hub 205014 and sharp 209114 configured to not bias the sensor 11900 prior to delivery, for example, during shipping and storage (FIG. 15B) and bias the sensor 11900 during delivery of the sensor (FIG. 37B). By storing and shipping the sensor in the unbiased (relaxed or unstressed) position, the sensor can have increased shelf life and lower overall stress. Furthermore, by storing and shipping the sensor in the unbiased position, stress relaxation over shelf life can be reduced and therefore loss in bias force due to stress relaxation can be limited. Accordingly, bias forces during delivery of the sensor can be more predictable and biasing during delivery can be as designed. The sharp 209114 can include a window 209114A. Prior to use, window 209114A can be aligned with protrusion 11912 on top end 11908b of the sensor 11900, and protrusion 11912 can extend through window 209114. In such a configuration, bottom end 11908a is not biased toward the sharp, and accordingly, sensor 11900 can be in a relaxed state. During firing, needle carrier 201102 can be partially retracted, thereby pulling sharp 209114 into a partially retracted position. Partial retraction can occur as the sheath 20704 initially moves proximally relative to the sensor carrier 20710 during firing. Each sharp carrier lock arm 20710K of the sensor carrier 20710 (see FIG. 9D) can extend radially outward as the rib 20710M of the retention arm 20710L engages a respective slot 20704Q of sheath 20704 (see FIG. 8M) which can allow sharp carrier retention feature 20710L to clear the pre-partial retraction retention face 201102A and engage the post-partial retraction retention face 201102B of the sharp carrier 201102 (see FIG. 10C). In the partially retracted position, window 209114A no longer receives protrusion 11912, and sharp 209114 engages protrusion 11912 to thereby biases the bottom end 11908a toward the sharp 209114 and into a proper position for delivery, as described above.

### Example Embodiments of Firing Mechanism of One-Piece and Two-Piece Applicators

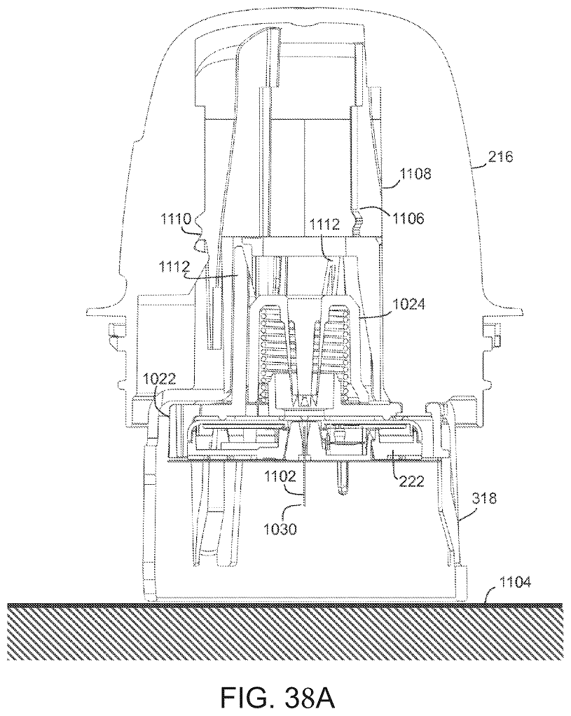

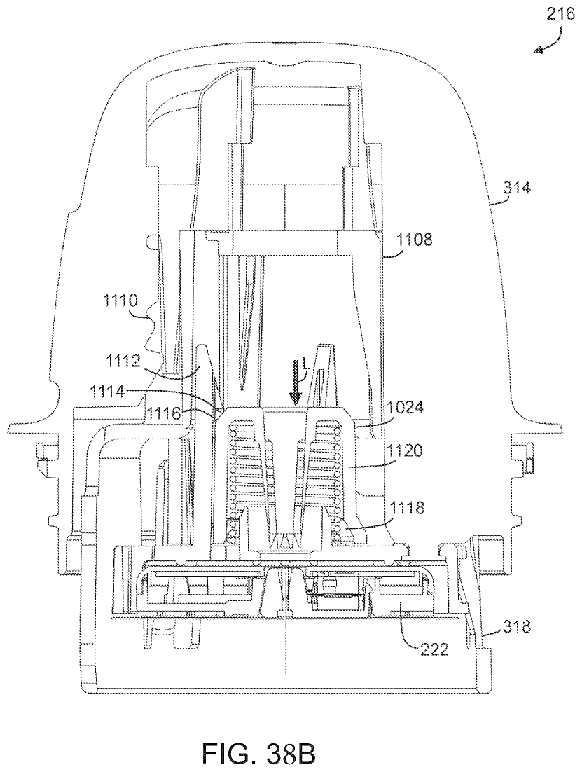

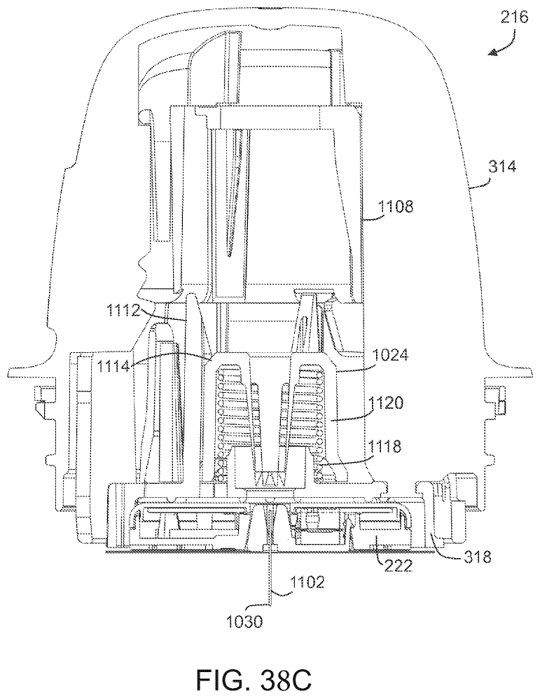

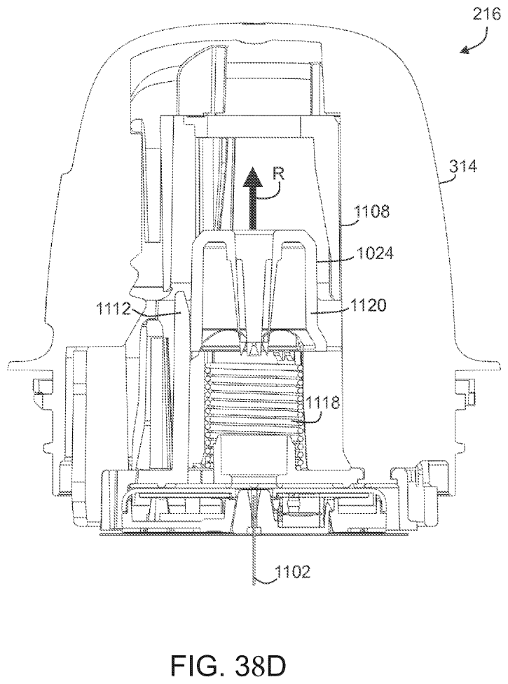

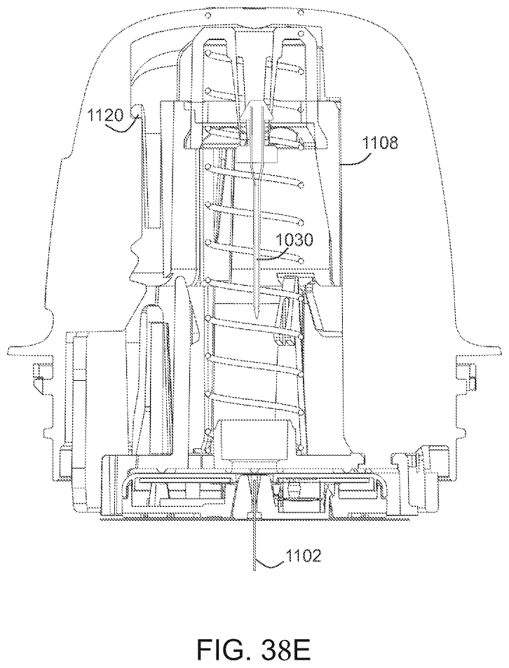

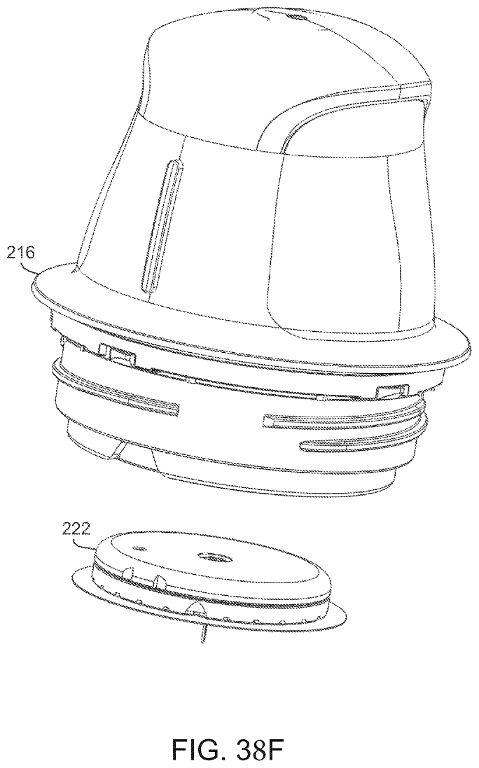

FIGS. 38A-38F illustrate example details of embodiments of the internal device mechanics of “firing” the applicator 216 to apply sensor control device 222 to a user and including retracting sharp 1030 safely back into used applicator 216. All together, these drawings represent an example sequence of driving sharp 1030 (supporting a sensor coupled to sensor control device 222) into the skin of a user, withdrawing the sharp while leaving the sensor behind in operative contact with interstitial fluid of the user, and adhering the sensor control device to the skin of the user with an adhesive. Modification of such activity for use with the alternative applicator assembly embodiments and components can be appreciated in reference to the same by those with skill in the art. Moreover, applicator 216 may be a sensor applicator having one-piece architecture or a two-piece architecture as disclosed herein.

Turning now to FIG. 38A, a sensor 1102 is supported within sharp 1030, just above the skin 1104 of the user. Rails 1106 (optionally three of them) of an upper guide section 1108 may be provided to control applicator 216 motion relative to sheath 318. The sheath 318 is held by detent features 1110 within the applicator 216 such that appropriate downward force along the longitudinal axis of the applicator 216 will cause the resistance provided by the detent features 1110 to be overcome so that sharp 1030 and sensor control device 222 can translate along the longitudinal axis into (and onto) skin 1104 of the user. In addition, catch arms 1112 of sensor carrier 1022 engage the sharp retraction assembly 1024 to maintain the sharp 1030 in a position relative to the sensor control device 222.

In FIG. 38B, user force is applied to overcome or override detent features 1110 and sheath 318 collapses into housing 314 driving the sensor control device 222 (with associated parts) to translate down as indicated by the arrow L along the longitudinal axis. An inner diameter of the upper guide section 1108 of the sheath 318 constrains the position of carrier arms 1112 through the full stroke of the sensor/sharp insertion process. The retention of the stop surfaces 1114 of carrier arms 1112 against the complimentary faces 1116 of the sharp retraction assembly 1024 maintains the position of the members with return spring 1118 fully energized.

In FIG. 38C, sensor 1102 and sharp 1030 have reached full insertion depth. In so doing, the carrier arms 1112 clear the upper guide section 1108 inner diameter. Then, the compressed force of the coil return spring 1118 drives angled stop surfaces 1114 radially outward, releasing force to drive the sharp carrier 1102 of the sharp retraction assembly 1024 to pull the (slotted or otherwise configured) sharp 1030 out of the user and off of the sensor 1102 as indicated by the arrow R in FIG. 38D.

With sharp 1030 fully retracted as shown in FIG. 38E, the upper guide section 1108 of sheath 318 is set with a final locking feature 1120. As shown in FIG. 38F, the spent applicator assembly 216 is removed from the insertion site, leaving behind the sensor control device 222, and with the sharp 1030 secured safely inside the applicator assembly 216. The spent applicator assembly 216 is now ready for disposal.

Operation of the applicator 216 when applying the sensor control device 222 is designed to provide the user with a sensation that both the insertion and retraction of the sharp 1030 is performed automatically by the internal mechanisms of the applicator 216. In other words, the present invention avoids the user experiencing the sensation that he is manually driving the sharp 1030 into his skin. Thus, once the user applies sufficient force to overcome the resistance from the detent features of the applicator 216, the resulting actions of the applicator 216 are perceived to be an automated response to the applicator being “triggered.” The user does not perceive that he is supplying additional force to drive the sharp 1030 to pierce his skin despite that all the driving force is provided by the user and no additional biasing/driving means are used to insert the sharp 1030. As detailed above in FIG. 38C, the retraction of the sharp 1030 is automated by the coil return spring 1118 of the applicator 216.

### Example Embodiments of Adhesive Liners

In many embodiments, an adhesive is disposed on a distal surface of a sensor control device, the adhesive being configured to secure the sensor control device to the user's skin. In such embodiments, it can be advantageous to utilize an adhesive liner to provide a protective barrier for the adhesive prior to the application of the sensor control device to the user's skin. Such adhesive liners can enhance the shelf life of the sensor control device.

Example embodiments of adhesive liners, as well as systems and methods for their removal will now be described. According to some embodiments, the adhesive liners can be removed upon removal of the applicator cap (e.g., FIG. 4B). This feature can reduce or eliminate the need for additional components and assembly steps. Additionally, this feature helps prevent users from inadvertently applying the adhesive patch without removing the adhesive liner.

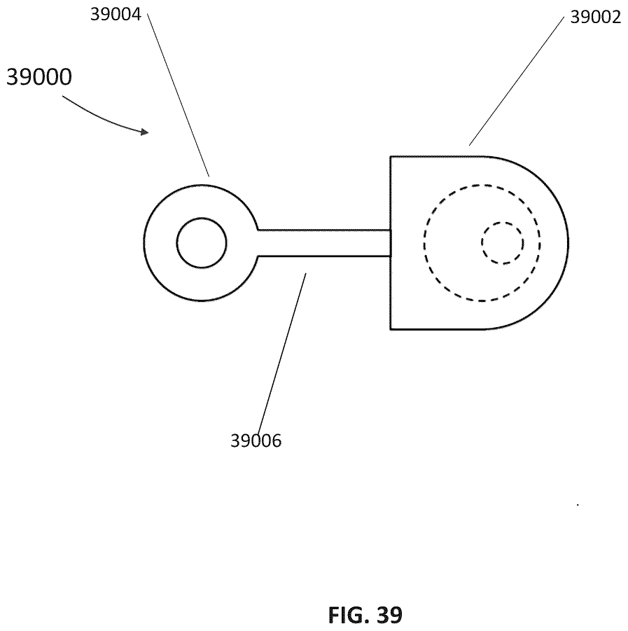

FIG. 39 depicts an example embodiment of an adhesive liner 39000. As shown in FIG. 39, adhesive liner 39000 can comprise a proximal part 39002 and a distal part 39004. Proximal part 39002 can be removably coupled with an adhesive patch. In some embodiments, proximal part 39002 can comprise a geometry that is at least partially complementary to the geometry of the adhesive patch (as indicated by the dashed lines). The adhesive patch (not shown) includes a distally facing surface that can be adhered to the skin of a user (see adhesive patch 20105 in FIG. 4F). As seen in FIG. 39, adhesive liner 39000 can also comprise a tail portion 39006, which connects proximal part 39002 and distal part 39004.

FIG. 40 depicts an example embodiment of an applicator assembly 40000 for insertion of an analyte sensor, wherein the applicator assembly includes an adhesive liner. Applicator assembly 40000 can comprise an applicator 40150, which can comprise applicator housing 40702, carrier 40710, a sharp (not shown), applicator cap 40708, which can be removably coupled with applicator housing 40702, and desiccant 40502, which can be disposed in applicator cap 40708. Carrier 40710 can releasably retain sensor control device 40102. In some embodiments, as shown in FIGS. 2B-2C, sensor control device 40102 can include a sensor control device housing, sensor electronics disposed in the sensor control device housing, an analyte sensor, which includes a proximal portion coupled with the sensor electronics, and a distal portion that can be transcutaneously positioned through the skin of the user. In many embodiments, the analyte sensor can be a glucose sensor. As shown in FIGS. 29A-B, in some embodiments, sensor control device 40102 can further include a sensor cap configured to cover the analyte sensor and the sharp, and the sensor cap can be removably coupled with the sensor control device housing. According to an aspect of the embodiments, the sensor cap can be removed along with the removal of the applicator cap 40708, such as described with respect to FIG. 35.

In many embodiments, sensor control device 40102 further includes an adhesive patch, which can have a distally-facing surface that is adhered to the skin of the user (see adhesive patch 20105 in FIG. 4F). Sensor control device 40102 can further include adhesive liner 39000, as depicted in FIG. 39. As seen in FIGS. 39-40, adhesive liner 39000 can include proximal part 39002, distal part 39004, and tail portion 39006. Proximal part 39002 can be removably coupled with the adhesive patch. Desiccant 40502 can be used to secure distal part 39004 of adhesive liner 39000 in applicator cap 40708. In some embodiments, distal part 39004 of adhesive liner 39000 cam comprise a geometry that is at least partially complementary to the geometry of desiccant 40502. As shown in FIG. 39, in some embodiments, distal part 39004 of adhesive liner 39000 can comprise a circular geometry. In some embodiments, distal part 39004 of adhesive liner can comprise other shapes, including rectangular shape, and irregular shapes. The sensor cap is in a spaced relationship with distal part 39004 of adhesive liner 39000 prior to removal of applicator cap 40708. Proximal part 39002 of adhesive liner 39000 is coupled with the adhesive patch prior to removal of applicator cap 40708, and during shipping and storage. When a user removes applicator cap 40708, distal part 39004 of adhesive liner 39000 can move along with applicator cap 40708, causing removal of proximal part 39002 of adhesive liner 39002 from the adhesive patch. In some embodiments, tail portion 39006 can be configured to fold like an accordion to facilitate tidy folding and prevent interference with the sensor cap. In other embodiments, tail portion 39006 can be made of an elastic material to prevent interference with the sensor cap. In some embodiments, the length of tail portion 39006 of adhesive liner 39000 can be sufficient to maintain the parts in place while being short enough to prevent the user from inserting the sensor without removing adhesive liner 39000.

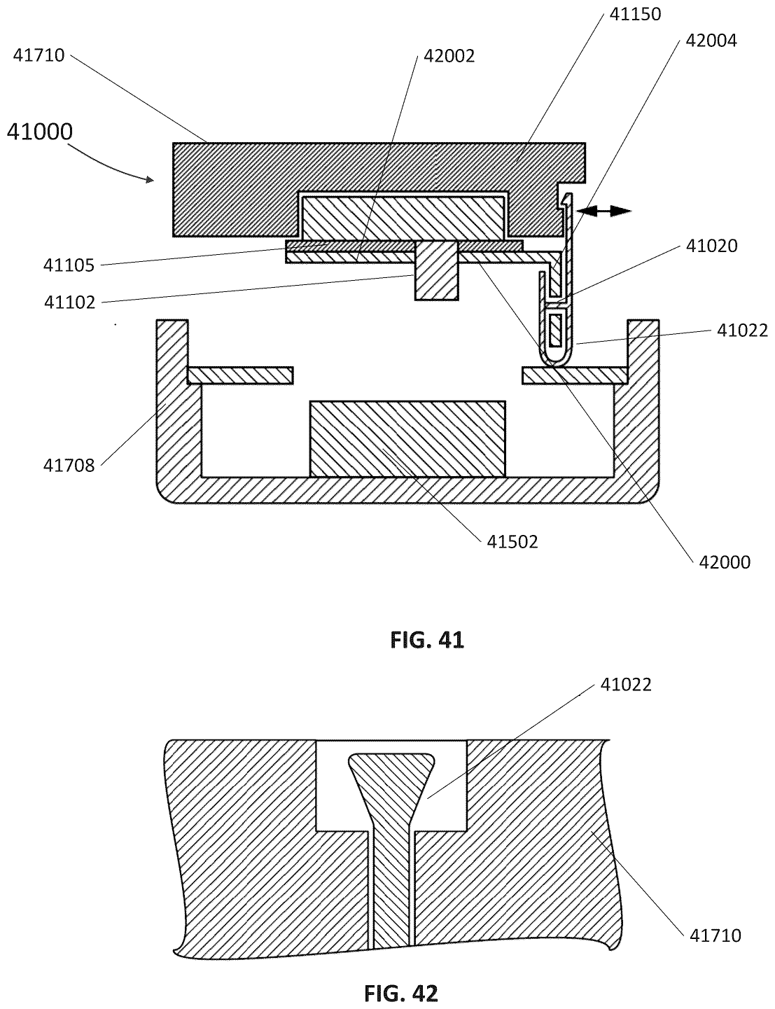

FIGS. 41-42 depict another example embodiment of an applicator assembly 41000 for insertion of an analyte sensor, wherein the assembly 41000 comprises an adhesive liner 42000. In many embodiments, applicator assembly 41000 comprises an applicator 41150, which can comprise an applicator housing (not shown), carrier 41710, a sharp (not shown), applicator cap 41708, which can be removably coupled with the applicator housing, and desiccant 41502, which can be disposed in applicator cap 41708. Carrier 41710 can releasably retain sensor control device 41102. In some embodiments, as shown in FIGS. 2B-2C, sensor control device 41102 can include a sensor control device housing, sensor electronics disposed in the sensor control device housing, an analyte sensor, which includes a proximal portion coupled with the sensor electronics, and a distal portion that can be transcutaneously positioned through the skin of the user. In some embodiments, the analyte sensor can be a glucose sensor.

According to another aspect of the embodiments, sensor control device 41102 further includes an adhesive patch 41105, which can include a distally-facing surface that is adhered to the skin of the user. Sensor control device 41102 further includes adhesive liner 42000. As shown in FIG. 41, adhesive liner 42000 comprises a proximal part 42002 and a distal part 42004. Proximal part 42002 of adhesive liner 42000 can be removably coupled with adhesive patch 41105. As shown in FIG. 41, applicator cap 41708 can include retention element 41022 and distal part 42004 of adhesive liner 42000 can include engagement element 41020. In many embodiments, engagement element 41020 can mate with retention element 41022. According to an aspect of the embodiments, when a user removes applicator cap 41708, distal part 42004 of adhesive liner 42000 can move along with applicator cap 41708, causing removal of proximal part 42002 of adhesive liner 42000 from adhesive patch 41105. In some embodiments, retention element 41022 can include a clip and engagement element 41020 can include a ring and the clip may interlock with the ring. In many embodiments, proximal part 42002 of the adhesive liner 42000 can be coupled with the adhesive patch during shipping and storage.

According to some embodiments, adhesive liners may be removed upon removal of the sensor cap. Since sensor caps can remain static during assembly, allowing inspection of liner attachment, it can be advantageous to base the liner removal process on the sensor cap.

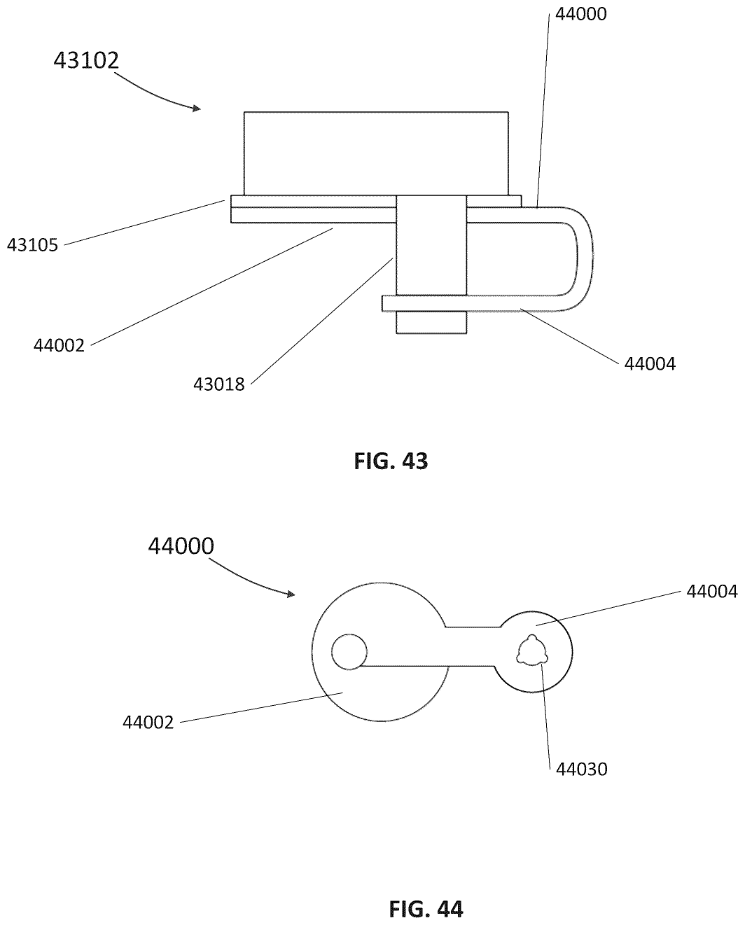

For example, FIG. 43 depicts an example embodiment of sensor control device 43102, wherein the sensor control device 43102 comprises adhesive liner 44000. In many embodiments, as shown in FIGS. 2B-2C, sensor control device 43102 comprises a sensor control device housing, sensor electronics disposed in the sensor control device housing, an analyte sensor, which can include a proximal portion coupled with the sensor electronics, and a distal portion that can be transcutaneously positioned through the skin of the user. In many embodiments, the analyte sensor can be a glucose sensor. In many embodiments, sensor control device 43102 can further include sensor cap 43018 configured to cover the analyte sensor and the sharp, and sensor cap 43018 can be removably coupled with the sensor control device housing. In some embodiments, sensor cap 43018 can be removed along with the removal of the applicator cap, such as described with respect to FIG. 35.

As shown in FIG. 43, sensor control device 43102 can include adhesive patch 43105, which comprises a distally-facing surface that is adhered to the skin of the user. In many embodiments, sensor control device 43102 can further include adhesive liner 44000. As shown in FIGS. 43-44, adhesive liner 44000 can include proximal part 44002 and distal part 44004. Proximal part 44002 of adhesive liner 44000 can be removably coupled with adhesive patch 43105. As shown in FIG. 44, proximal part 44002 of adhesive liner 44000 can comprise a geometry that is at least partially complementary to the geometry of adhesive patch 43105. In some embodiments, distal part 44004 of adhesive liner 44000 can include aperture 44030 to receive sensor cap 43018, as seen in FIG. 43. In this way, adhesive liner 44000 can be removed upon removal of sensor cap 43018 along with the applicator cap.

According to an aspect of the embodiments, aperture 40030 can be sized and shaped similarly (e.g., keyhole-shaped) to sensor cap 43018, such that distal part 44004 of adhesive liner 44000 can couple with sensor cap 43018, as shown in FIG. 44. In some embodiments, the adhesive liner removal process may not start until sensor cap 43018 is removed from sensor control device 43102. In many embodiments, adhesive liner 44000 may be coupled with adhesive patch 43105 during shipping and storage.

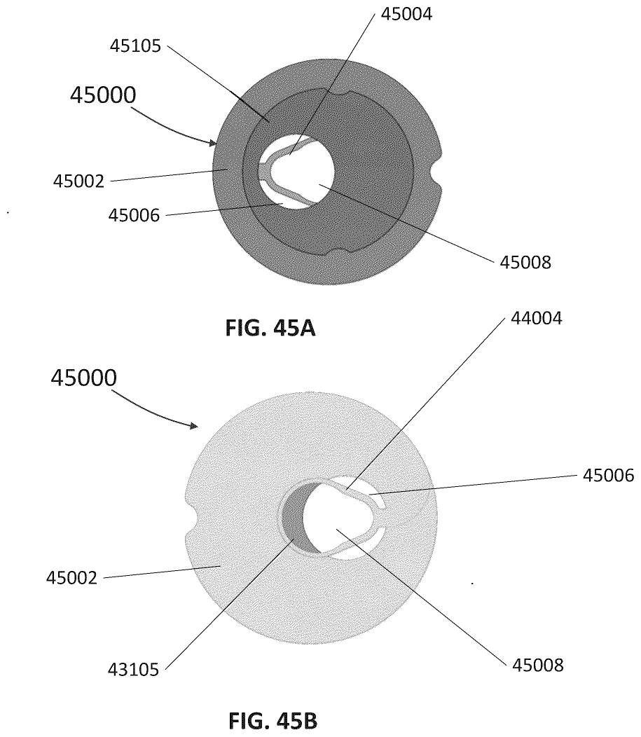

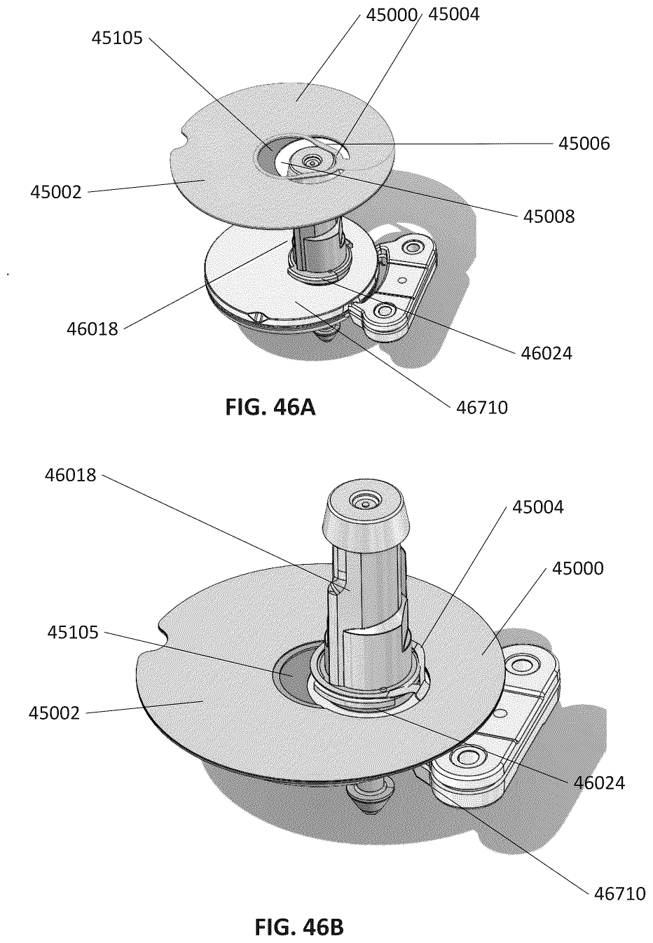

FIGS. 45A-45B depict an example embodiment of adhesive liner 45000. As shown in FIGS. 45A-45B, adhesive liner 45000 includes proximal part 45002 and distal part 45004. Proximal part 45002 of adhesive liner 45000 can be removably coupled with adhesive patch 45105, as depicted in FIG. 45A. Adhesive patch 45105 can include an aperture 45006. Distal part 45004 of adhesive liner 45000 can also include an aperture 45008. As shown in FIGS. 45A-45B, aperture 45008 differs in shape and size from aperture 45006, such that they do not substantially overlap. In many embodiments, aperture 45008 has a smaller diameter than aperture 45006, facilitating the coupling of distal part 45004 of adhesive liner with sensor cap 46018, as shown in FIGS. 46A-46B. In some embodiments, as depicted in FIGS. 46A-46B, as adhesive patch 45105 and adhesive liner 45000 are being assembled onto carrier 46710, aperture 45008 of distal part 45004 of adhesive liner 45000 can mate with a narrow and tapered surface of sensor cap plug 46024. As adhesive patch 45105 is pressed down to its final position, distal part 45004 of adhesive liner 45000 can be engaged with the sensor cap plug 46024. As such, removing sensor cap 46018 will cause removal of adhesive liner 45000 from adhesive patch 45105.

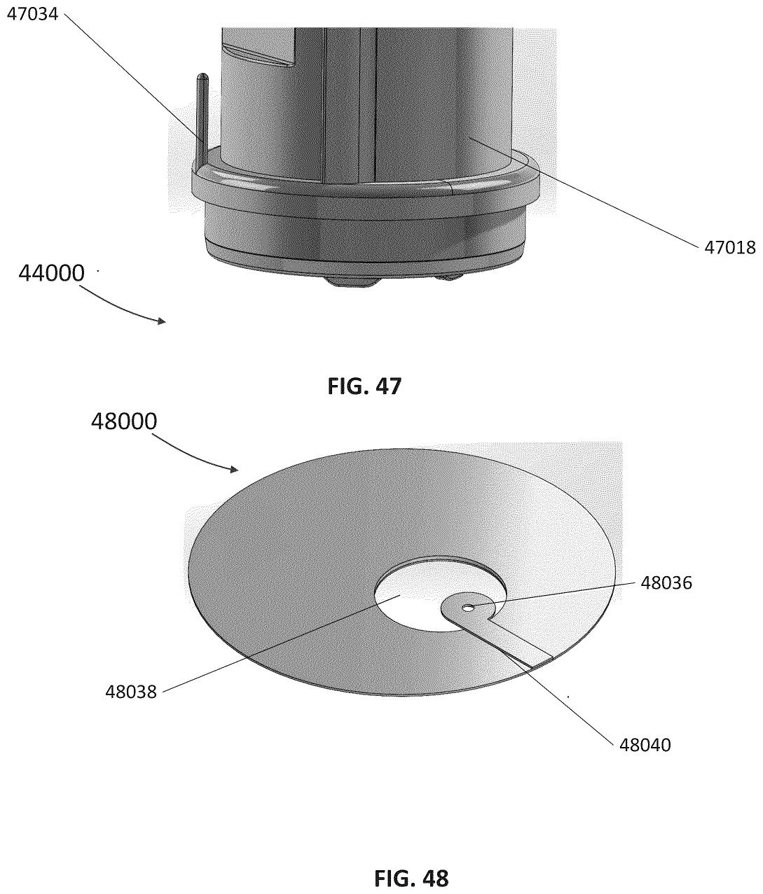

FIG. 47 depicts an example embodiment of a sensor cap 47018. As shown in FIG. 47, sensor cap 47018 can have retention element 47034 to mate with an engagement element in the adhesive liner. As such, removing of sensor cap 47018 can cause removal of the adhesive liner from the adhesive patch. As shown in FIG. 47, in some embodiments, retention element 47034 can be disposed on a side of sensor cap 47018, wherein retention element 47034 extends in a direction that is parallel to the direction of the sharp. In some embodiments, retention element 47034 can include a protrusion configured to mate with the engagement element in the adhesive liner. In other embodiments, retention element 47034 can be a post configured to mate with the engagement element in the adhesive liner.

FIG. 48 depicts an example embodiment of an adhesive liner 48000. In many embodiments, adhesive liner 48000 can be removably coupled with the adhesive patch. As shown in FIG. 48, adhesive liner 48000 can include engagement element 48036 to mate with retention element 47034 of sensor cap 47018, shown in FIG. 47. In some embodiments, engagement element 48036 can include an aperture, which can mate with retention element 47034. According to an aspect of the embodiments, adhesive liner 48000 may further include split line 48040 to facilitate adhesive liner removal. In some embodiments, split line 48040 can be located adjacent to engagement element 48036. In many embodiments, split line 40040 can be created through kiss cut. According to another aspect of the embodiments, adhesive liner 48000 comprises aperture 48038 that is at least partially complementary to the geometry of the adhesive patch.

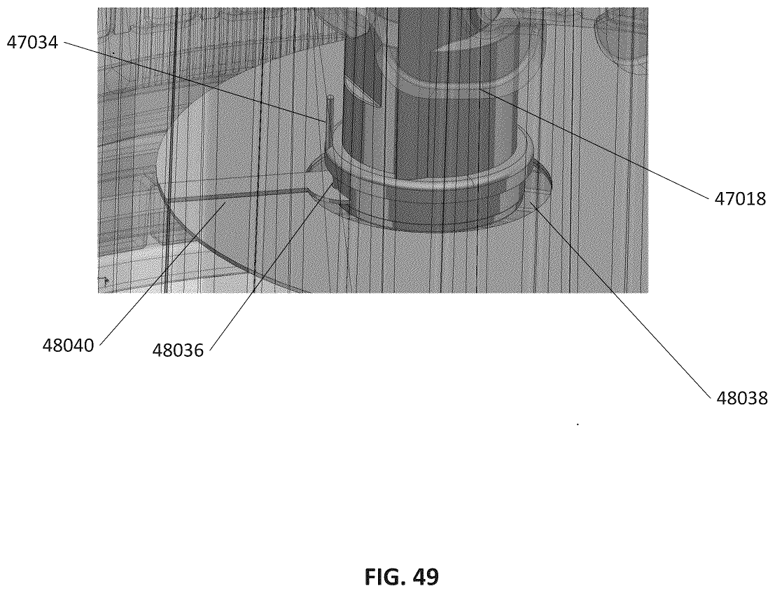

FIG. 49 provides a perspective view of the sensor cap 47018 in FIG. 47 coupled with the adhesive liner 48000 of FIG. 48. As shown in FIG. 49, engagement element 48036 of adhesive liner 48000 can mate retention element 47034 of sensor cap 47018, such that removal of sensor cap 47018 and the applicator cap (not shown) can cause removal of adhesive liner 48000 from the adhesive patch. In some embodiments, adhesive liner 48000 can be removed in a clockwise direction upon removal of sensor cap 47018. In other embodiments, adhesive liner 48000 can be removed in a counterclockwise direction upon removal of sensor cap 47018.

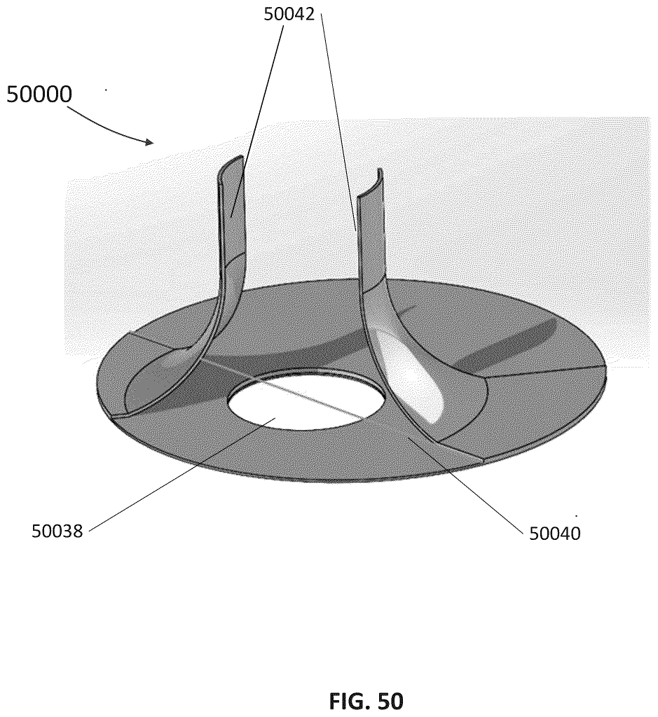

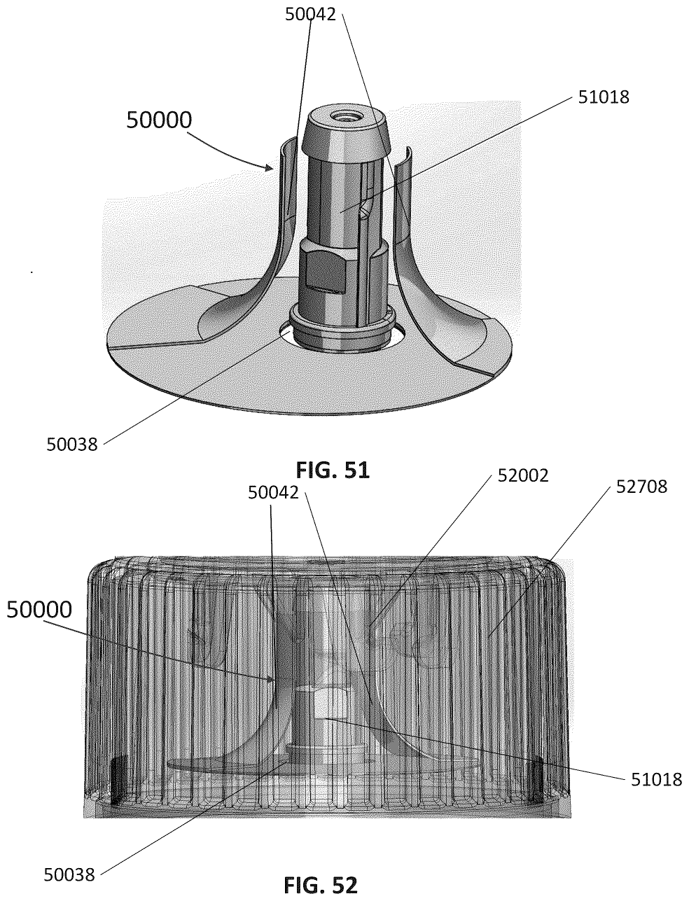

FIG. 50 depicts another example embodiment of an adhesive liner 50000. As shown in FIG. 50, adhesive liner 50000 can comprise a plurality of liner tabs 50042. In some embodiments, the plurality of liner tabs 50042 can include two liner tabs. In many embodiments, adhesive liner 50000 comprises a surface that can be removably coupled with the adhesive patch. In some embodiments, liner tabs 50042 can be substantially perpendicular to the surface that is coupled with the adhesive patch. In many embodiments, adhesive liner 50000 can have aperture 50038 to receive sensor cap 40026 (see FIG. 51). In many embodiments, adhesive liner 50000 may further include split line 50040 to facilitate adhesive liner removal. In some embodiments, split line 50040 can be created by kiss cut adhesive liner 50000. In some embodiments, split line 50040 can be adjacent to the first liner tab of plurality of liner tabs 50042. In other embodiments, split line 50040 can be adjacent to the second liner tab of plurality of liner tabs 40042. In other embodiments, adhesive liner 50000 can include a plurality of split lines that are adjacent to each of the plurality of liner tabs.

As depicted in FIG. 51, a first liner tab of plurality of liner tabs 50042 can be coupled with sensor cap 51018 at a first position or on a first side, and a second liner tab of plurality of liner tabs 50042 can be coupled with sensor cap 51018 at a second position or on a second side, such that adhesive liner 50000 can be removed upon removal of sensor cap 51018. In some embodiments, the first side or position may be substantially opposite the second side or position. In some embodiments, liner tabs 50042 can be secured to sensor cap 40026 using a pressure curing adhesive. In other embodiments, as shown in FIG. 52, ratchet features 52002 on applicator cap 52708 can further secure liner tabs 50042 onto sensor cap 51018. In some embodiments, adhesive liner 50000 can be removed in a clockwise direction upon removal of sensor cap 51018. In other embodiments, adhesive liner 50000 can be removed in a counterclockwise direction upon removal of sensor cap 51018.

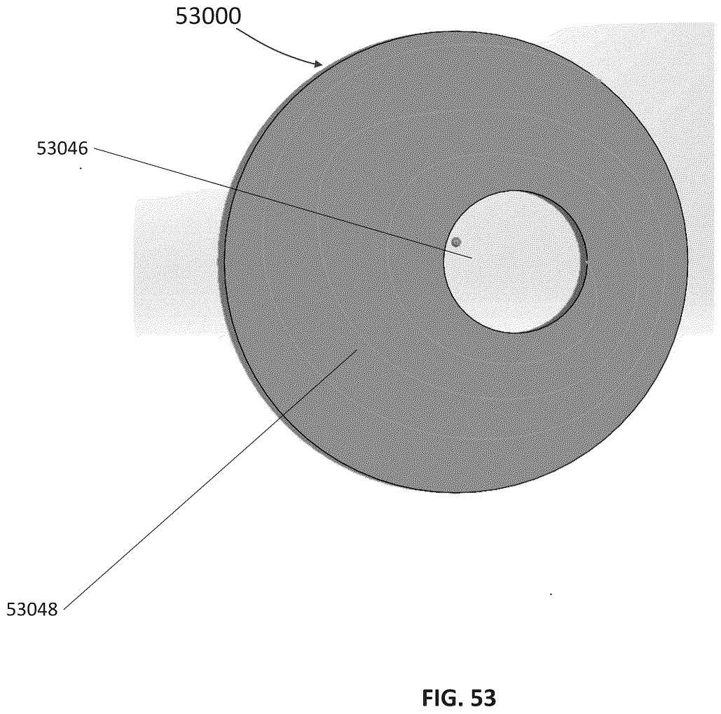

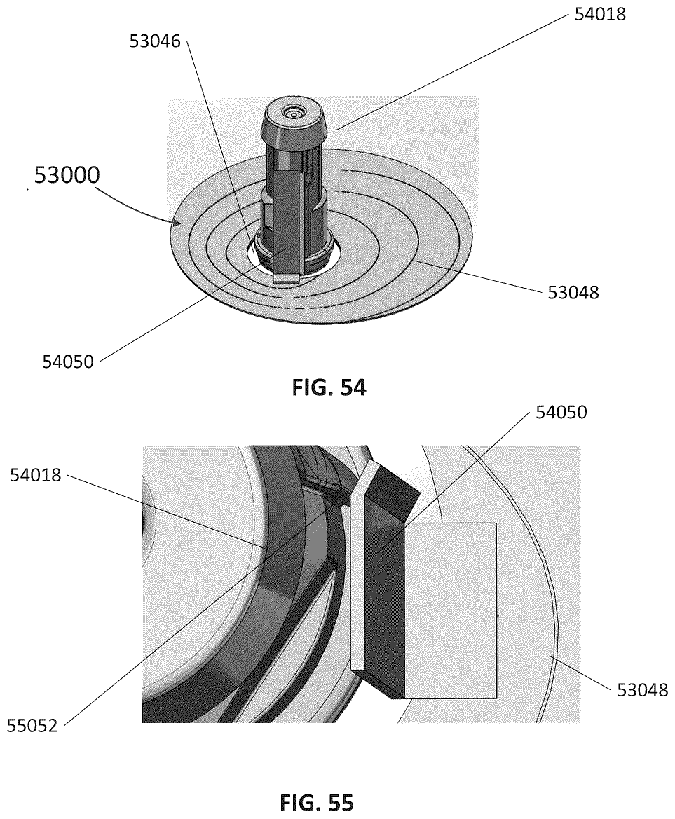

FIG. 53 depicts another example embodiment of an adhesive liner. In many embodiments, as shown in FIG. 53, adhesive liner 53000 can include spiral pattern 53048 to facilitate adhesive liner removal. As depicted in FIGS. 53-54, in some embodiments, adhesive liner 53000 can include aperture 53046 to receive sensor cap 54026. As shown in FIGS. 54-55, adhesive liner 53000 can further include tab 54050, which can couple with ratchet feature 55052 in sensor cap 54018. Adhesive liner 53000 can comprise a surface that can be removably coupled with the adhesive patch. In some embodiments, tab 54050 can be substantially perpendicular to the surface that is coupled with the adhesive patch. As such, removal of sensor cap 54018 can cause removal of adhesive liner 53000 from the adhesive patch. In many embodiments, tab 54050 can be secured to sensor cap 54018 using adhesives. In some embodiments, ratchet features of the applicator cap can further secure tab 54050 to ratchet feature 55052 of sensor cap 54018, reducing the need for adhesive action. According to another aspect of the embodiments, instead of kiss cutting the adhesive liners, adhesives may be kiss cut instead. In some embodiments, it is preferred to kiss cut the adhesives before applying the adhesive liners to the adhesive patch.

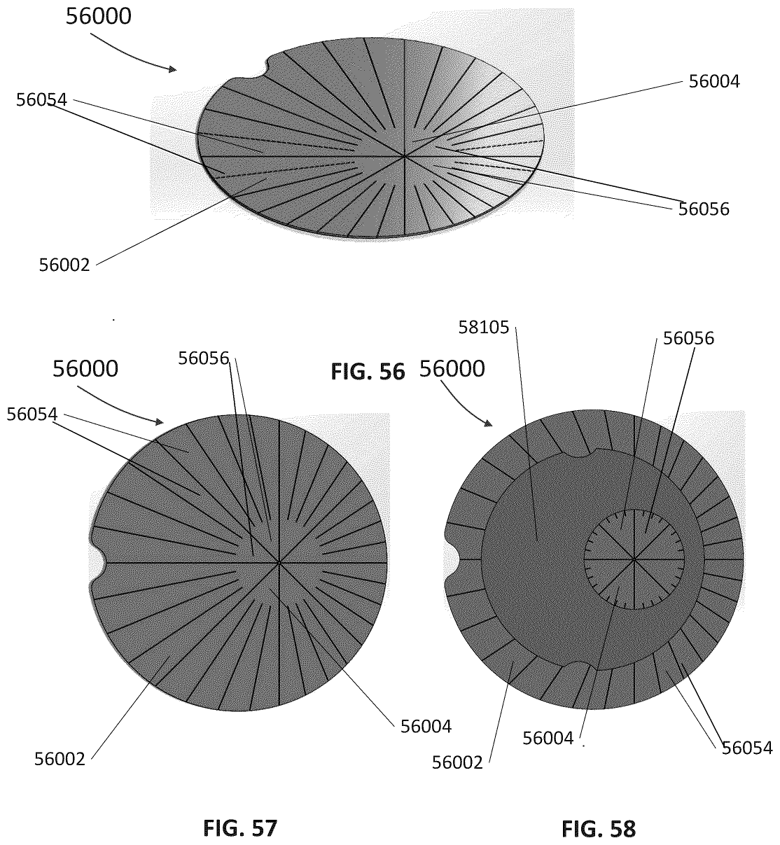

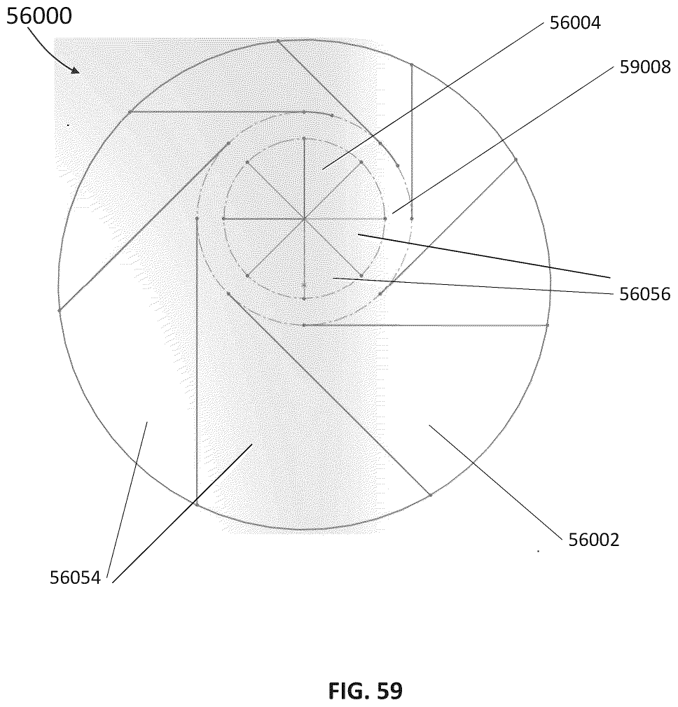

FIGS. 56-59 depict example embodiments of adhesive liners. According to some embodiments, adhesive liner 56000 can have an outer portion 56002 and a center portion 56004. As shown in FIGS. 56-59, outer portion 56002 can be kiss cut into a plurality of segments 56054. In some embodiments, each of the plurality of segments 56054 of outer portion 56002 can have a substantially triangular shape, as shown in FIG. 56-58. In other embodiments, each of the plurality of segments 56054 of outer portion 56002 can have a substantially spiral shape, as shown in FIG. 59. In many embodiments, center portion 56004 can also be kiss cut into a plurality of segments 56056, as depicted in FIGS. 56-59. In some embodiments, each of the plurality of segments 45056 of center portion 56004 can have a substantially triangular shape, as shown in FIGS. 56-59.

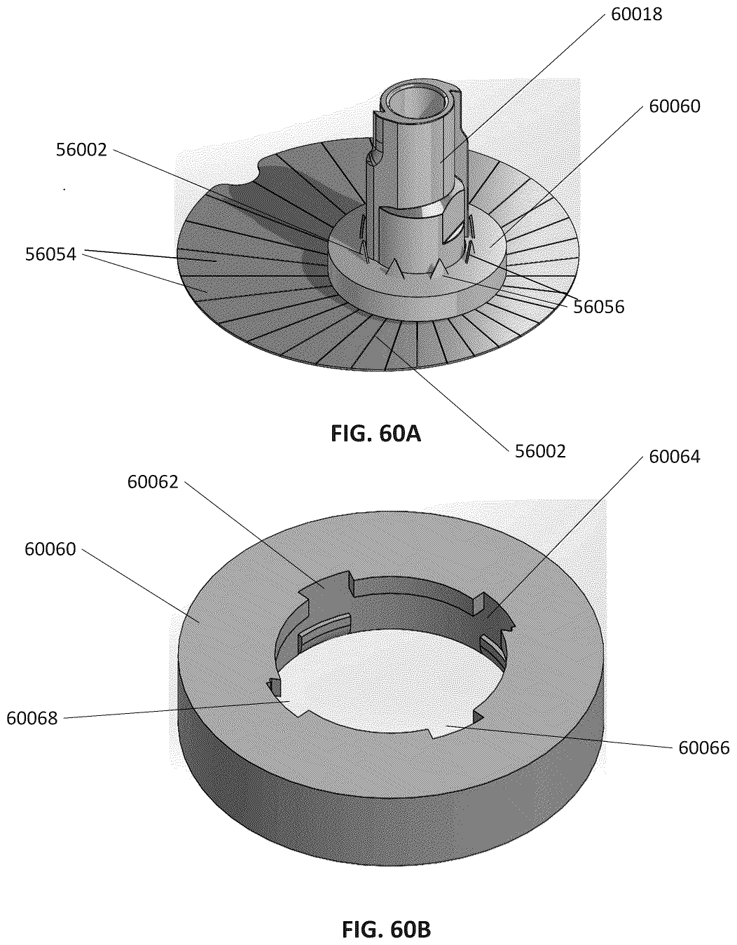

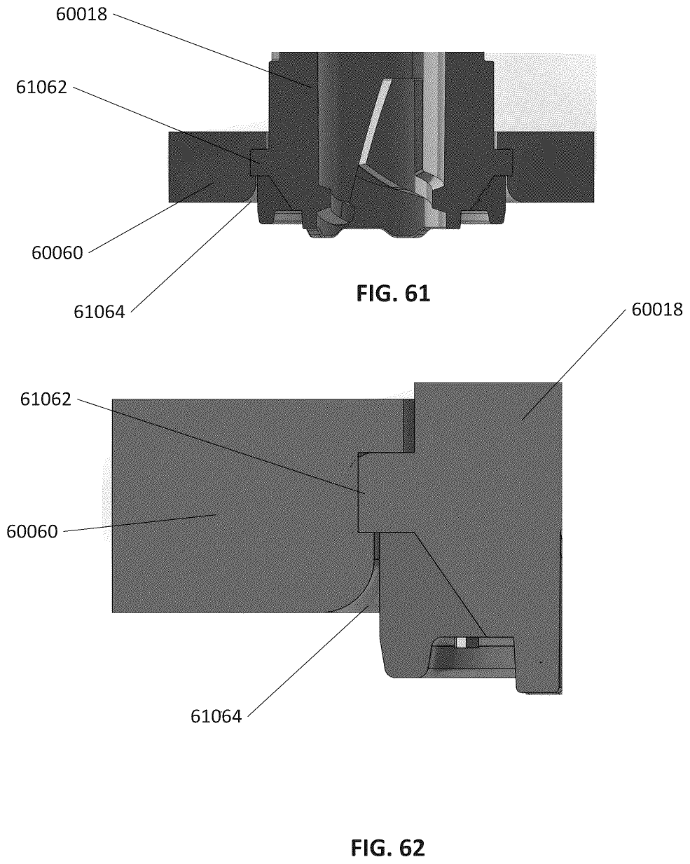

As shown in FIG. 58, adhesive liner 56000 can be coupled with adhesive patch 58105. Adhesive patch can comprise an aperture 58024 to receive the sensor cap. As shown in FIG. 58, center portion 56004 of adhesive liner 56000 substantially overlaps with aperture 58024 of adhesive patch. In some embodiment, center portion 56004 of adhesive liner 56000 is complimentary to aperture 58024 of adhesive patch. As shown in FIG. 60A, in many embodiments, each of plurality of segments 40055 of center portion 56004 of adhesive liner 56000 can be captured between sensor cap 60018 and collar 60060 individually. As shown in FIG. 60B, collar 60060 can include a plurality of dents. In some embodiments, the plurality of dents can include four dents, 60062, 60064, 60066, and 60068, as shown in FIG. 60B. In some embodiments, as shown in FIG. 60B, collar 60060 is “A-B moldable” because it consists of two main mold halves that can separate to release the part without complex mechanisms. This design simplifies the molding process, reduces production costs, and minimizes potential defects, making it more efficient and cost-effective to manufacture. As shown in FIGS. 61-62, collar 60060 can be pressed over sensor cap 60018 and snaps into place. According to another aspect of the embodiments, sensor cap 60018 can include ring 61062, and collar 60060 can be crimped onto ring 61062 by engaging ring 61062 with the plurality of dents in collar 60060. As shown in FIGS. 61-62, the interface between collar 60060 and ring 61062 creates a gap 61064 for each of the plurality of segment 56056 of end portion 56004 of adhesive liner 56000 to pass through, securing them between collar 60060 and sensor cap 60018. As such, when the user removes the applicator cap, which causes sensor cap 600018 to rotate, and further causes removal of adhesive liner 56000 from the adhesive patch.

Referring back to FIG. 59, adhesive liner 56000 can further comprise a middle portion 59008, separating center portion 56004 from outer portion 56002. To prevent adhesive liner 56000 from being pulled off the adhesive patch while coupling the adhesive liner to sensor cap 60018 and collar 60060, it is advantageous for adhesive liner 56000 to have this uncut middle portion 59008 to separate center portion 56004 from outer portion 56002, thereby averting any potential buckling of adhesive liner 56000. Additionally, in the presence of middle portion 59008, if not all of the plurality of segment 56056 of end portion 56004 of adhesive liner 56000 are securely held between collar 60060 and sensor cap 60018, the turning force from twisting sensor cap 60018 is still effectively transmitted to all parts of adhesive liner 56000. According to some embodiments, middle portion 59008 can have a ring shape.

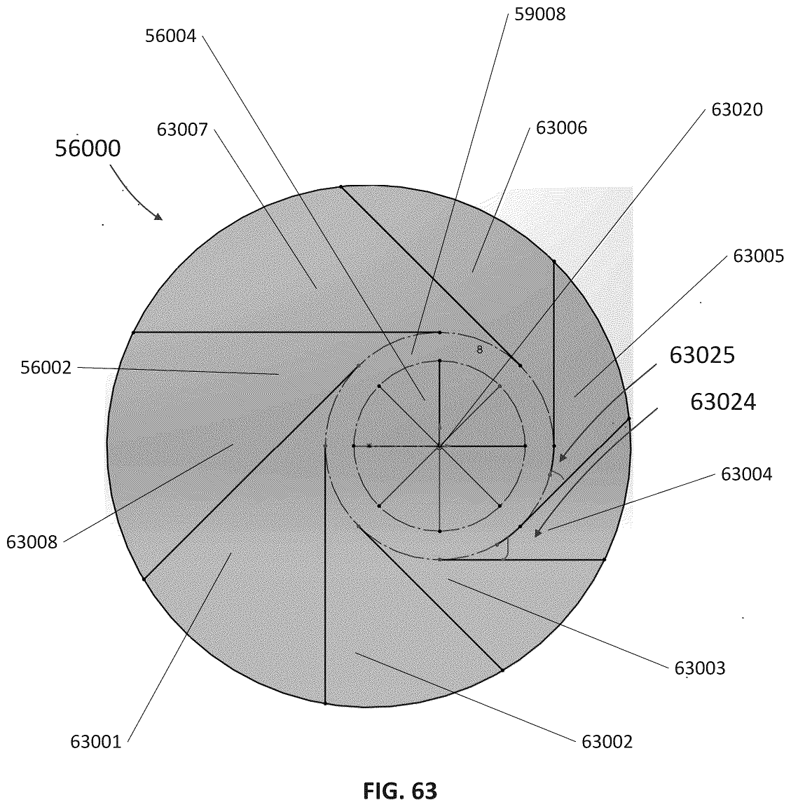

Referring now to FIG. 63, according to some embodiments the plurality of segments 56054 of outer portion 56002 can have eight (8) different segments, segments 63001 through 63008, via kiss cutting. In some embodiments, when a turning force is applied to center point 63020 to unscrew sensor cap 60018, segments 63001 through 63008 are configured to buckle, causing removal of adhesive liner 56000 from the adhesive patch. According to some embodiments, segments 63001 through 63008 are configured to buckle at the shortest distance to the nearest kiss cut. Specifically, as sensor cap 60018 is twisted counterclockwise and unscrewed, segments 63004, 63005, 63006, 63007, and 63008 are prone to compression, while segments 63001, 63002, and 63003 are prone to tension. Compression can cause wrinkles in adhesive liners, reducing adhesive effectiveness. To mitigate compression, according to some embodiments, the kiss cuts in the outer portion 56002 of adhesive liner 56000 are tangential to the middle portion 59008, and are designed to buckle outward, expanding the outer diameter of adhesive liner 56000. Simultaneously, the middle portion 59008 pulls each of segments 63001-63008 clockwise, generating a turning force that pushes adhesive liner 56000 outward, causing the outer portion 56002 to be in tension for all segments. As shown in FIG. 63, in some embodiments, segments 63004 and 63005 can extend approximately 15 degrees along the outer diameter of the middle portion 59008 to provide the necessary force for buckling (see angles 63024 and 63025).

In some embodiments, collar 60060 can comprise an elastomeric material, such that it does not require as much force to apply it to sensor cap 60018. According to some embodiments, sensor cap 60018 can comprise splines, facilitating the securement of the plurality of segment 56056 of end portion 56004 of adhesive liner 56000 between collar 60060 and sensor cap 60018. In some embodiments, adhesive liner 56000 can comprise siliconized paper. In other embodiments, adhesive liner 56000 can comprise siliconized polyester.

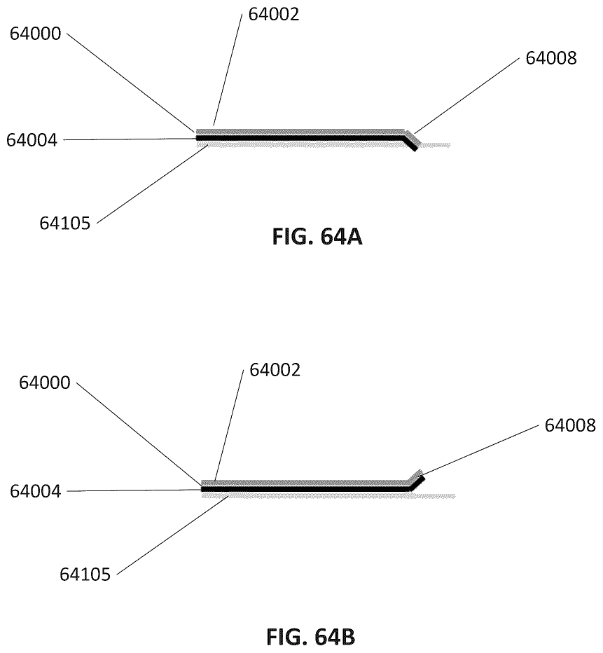

In many embodiments, as shown in FIGS. 64A-64B, burrs 64008 can remain following the kiss cutting process. As shown in FIGS. 64A-64B, adhesive liner 64000 can include two layers, a top layer 64002, and a bottom layer 64004. In some embodiments, top layer 64002 can comprise paper. In some embodiments, bottom layer 64004 can comprise silicone. According to some embodiments, it is preferable for burrs 64008 to be positioned away from the adhesive patch 64105, as depicted in FIG. 64B. When burrs 64008 are positioned towards the adhesive patch 64105, as shown in FIG. 64A, burrs 64008 can be pressed into the adhesives of the adhesive patch 64105, anchoring the kiss cut areas of the adhesive liner 64000 into the adhesives and therefore reducing the effectiveness of the adhesives.

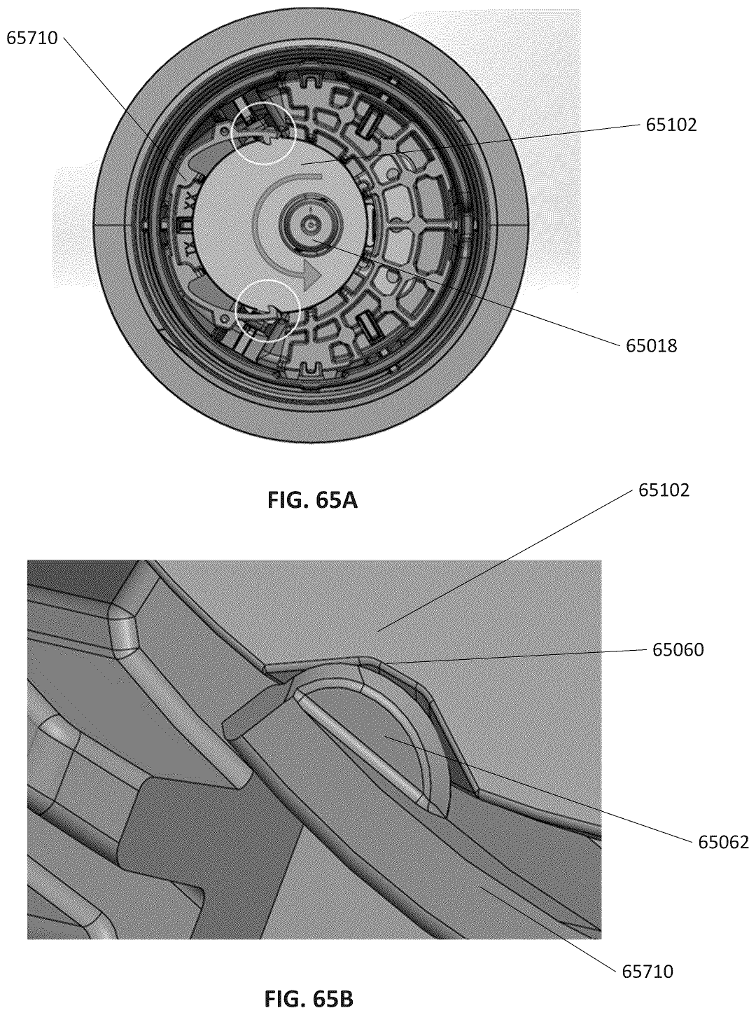

In many embodiments, as the adhesive liner is removed from the adhesive patch, sensor control device 65102 twists counterclockwise with sensor cap 65018, as shown in FIG. 65A. To prevent sensor control device 65102 from twisting out of the arms of carrier 65710, in some embodiments, flat section 65060 can be added to sensor control device 65102, and flat section 65062 can be added to arms of carrier 65710, as shown in FIG. 65B. This modification significantly increases the turning force required to strip the sensor control device from the carrier arms, thereby keeping the sensor control device in the carrier arms during adhesive liner removal.

Example embodiments of the devices described herein are set out in the following numbered clauses.

Clause 1. An assembly for insertion of an analyte sensor, the assembly comprising:
(1) an applicator comprising:
   an applicator cap;
(2) a sensor control device configured to be worn on skin of a user, the sensor control device comprising:
a sensor control device housing;
sensor electronics disposed in the sensor control device housing;
the analyte sensor;
an adhesive patch comprising a distally-facing surface configured to adhere to the skin of the user; and
an adhesive liner comprising:
a proximal part removably coupled with the adhesive patch; and
a distal part coupled with the applicator cap;
wherein the proximal part of adhesive liner is configured such that removal of the sensor cap and the applicator cap causes removal of the proximal part of the adhesive liner from the adhesive patch.

Clause 2. The assembly of clause 1, wherein the applicator further comprises a desiccant disposed in the applicator cap, and wherein the desiccant is configured to secure the distal part of the adhesive liner in the applicator cap.

Clause 3. The assembly of any of clauses 1-2, wherein the applicator cap comprises a retention element, wherein the distal part of the adhesive liner comprises an engagement element, and wherein the engagement element of the adhesive liner is configured to mate with the retention element of the applicator cap.

Clause 4. The assembly of any of clauses 1-3, wherein the applicator further comprises an applicator housing, a sharp, and a carrier configured to releasably retain the sensor control device, and wherein the applicator cap is removably coupled with the applicator housing.

Clause 5. The assembly of any of clauses 1-4, wherein the analyte sensor comprises a proximal portion coupled with the sensor electronics and a distal portion configured to be transcutaneously positioned through the skin of the user.

Clause 6. An assembly for insertion of an analyte sensor, the assembly comprising:
(1) an applicator comprising:
   an applicator cap;
(2) a sensor control device configured to be worn on skin of a user, the sensor control device comprising:
a sensor control device housing;
sensor electronics disposed in the sensor control device housing;
the analyte sensor;
a sensor cap configured to cover the analyte sensor and the sharp, wherein the sensor cap is removably coupled with the sensor control device housing, and wherein the sensor cap is further configured such that the removal of the applicator cap further causes removal of the sensor cap from the sensor control device housing;
an adhesive patch comprising a distally-facing surface configured to adhere to the skin of the user; and
an adhesive liner removably coupled with the adhesive patch;
wherein the adhesive liner is configured such that removal of the sensor cap and the applicator cap causes removal of the adhesive liner from the adhesive patch.

Clause 7. The assembly of clause 6, wherein the adhesive liner comprises a proximal part removably coupled with the adhesive patch and a distal part coupled with the sensor cap, and wherein the distal part of the adhesive liner comprises an aperture to receive the sensor cap.

Clause 8. The assembly of any of clauses 6-7, wherein the sensor cap comprises a retention element, wherein the adhesive liner comprises an engagement element, and wherein the engagement element of the adhesive liner is configured to mate with the retention element of the sensor cap.

Clause 9. The assembly of any of clauses 6-8, wherein the adhesive liner comprises a plurality of liner tabs, wherein a first liner tab of the plurality of liner tabs is coupled with the sensor cap at a first position, and wherein a second liner tab of the plurality of liner tabs is coupled with the sensor cap at a second position.

Clause 10. The assembly of any of clauses 6-9, wherein the sensor cap comprises a ratchet feature, wherein the adhesive liner comprises a spiral pattern and a liner tab, and wherein the liner tab of the adhesive liner is configured to mate with the ratchet feature of the sensor cap.

Clause 11. The assembly of any of clauses 6-10, wherein the applicator further comprises an applicator housing, a sharp, and a carrier configured to releasably retain the sensor control device, and wherein the applicator cap is removably coupled with the applicator housing.

Clause 12. The assembly of any of clauses 6-11, wherein the analyte sensor comprises a proximal portion coupled with the sensor electronics and a distal portion configured to be transcutaneously positioned through the skin of the user.

Clause 13. An assembly for insertion of an analyte sensor, the assembly comprising:
(1) an applicator, comprising:
   an applicator housing;
   a sharp;
   a carrier configured to releasably retain a sensor control device;
   an applicator cap removably coupled with the applicator housing; and
   a desiccant disposed in the applicator cap;
(2) the sensor control device configured to be worn on skin of a user, the sensor control device comprising:
a sensor control device housing;
sensor electronics disposed in the sensor control device housing;
the analyte sensor comprising:
a proximal portion coupled with the sensor electronics; and
a distal portion configured to be transcutaneously positioned through the skin of the user;
an adhesive patch comprising a distally-facing surface configured to adhere to the skin of the user; and
an adhesive liner comprising:
a proximal part removably coupled with the adhesive patch; and
a distal part coupled with the applicator cap;
wherein the desiccant is configured to secure the distal part of the adhesive liner in the applicator cap, and wherein the proximal part of the adhesive liner is configured such that removal of the applicator cap causes removal of the proximal part of the adhesive liner from the adhesive patch.

Clause 14. The assembly of clause 13, wherein the analyte sensor is a glucose sensor.

Clause 15. The assembly of any of clauses 13-14, wherein the proximal part of the adhesive liner is configured to be coupled with the adhesive patch during shipping and storage.

Clause 16. The assembly of any of clauses 13-15, wherein the sensor control device further comprises a sensor cap configured to cover the analyte sensor and the sharp, wherein the sensor cap is removably coupled with the sensor control device housing, and wherein the sensor cap is further configured such that the removal of the applicator cap further causes removal of the sensor cap from the sensor control device housing.

Clause 17. The assembly of any of clauses 13-16, wherein the sensor cap is in a spaced relationship with the distal part of the adhesive liner prior to the removal of the applicator cap.

Clause 18. The assembly of any of clauses 13-17, wherein the distal part of the adhesive liner comprises a circular geometry.

Clause 19. An assembly for insertion of an analyte sensor, the assembly comprising:
(1) an applicator comprising:
   an applicator housing;
   a sharp;
   a carrier configured to releasably retain a sensor control device; and
   an applicator cap removably coupled with the applicator housing, the applicator cap comprising a retention element;
(2) the sensor control device configured to be worn on skin of a user, the sensor control device comprising:
a sensor control device housing;
sensor electronics disposed in the sensor control device housing;
the analyte sensor comprising:
a proximal portion coupled with the sensor electronics; and
a distal portion configured to be transcutaneously positioned through the skin of the user;
an adhesive patch comprising a distally-facing surface configured to adhere to the skin of the user; and
an adhesive liner comprising:
a proximal part removably coupled with the adhesive patch; and
a distal part comprising an engagement element;
wherein the engagement element of the adhesive liner is configured to mate with the retention element of the applicator cap, and wherein the adhesive liner is configured such that removal of the applicator cap causes removal of the proximal part of the adhesive liner from the adhesive patch.

Clause 20. The assembly of clause 19, wherein the analyte sensor is a glucose sensor.

Clause 21. The assembly of any of clauses 19-20, wherein the proximal part of the adhesive liner is configured to be coupled with the adhesive patch during shipping and storage.

Clause 22. The assembly of any of clauses 19-21, wherein the retention element of the applicator cap comprises a protrusion configured to snap-fit into an aperture in the engagement element of the adhesive liner.

Clause 23. The assembly of any of clauses 19-22, wherein the engagement element of the adhesive liner comprises a ring and the retention element of the applicator cap comprises a clip, and wherein the clip is configured to interlock with the ring.

Clause 24. An assembly for insertion of an analyte sensor, the assembly comprising:
(1) an applicator comprising:
   an applicator housing;
   a sharp;
   a carrier configured to releasably retain a sensor control device; and
   an applicator cap removably coupled with the applicator housing;
(2) the sensor control device configured to be worn on skin of a user, the sensor control device comprising:
a sensor control device housing;
sensor electronics disposed in the sensor control device housing;
the analyte sensor comprising:
a proximal portion coupled with the sensor electronics; and
a distal portion configured to be transcutaneously positioned through the skin of the user;
a sensor cap configured to cover the analyte sensor and the sharp, wherein the sensor cap is removably coupled with the sensor control device housing, and wherein the sensor cap is further configured such that the removal of the applicator cap further causes removal of the sensor cap from the sensor control device housing;
an adhesive patch comprising a distally-facing surface configured to adhere to the skin of the user; and
an adhesive liner comprising:
a proximal part removably coupled with the adhesive patch; and
a distal part coupled with the sensor cap;
wherein the distal part of the adhesive liner comprises an aperture to receive the sensor cap, and wherein the adhesive liner is configured such that removal of the sensor cap and the applicator cap causes removal of the proximal part of the adhesive liner from the adhesive patch.

Clause 25. The assembly of clause 24, wherein the analyte sensor is a glucose sensor.

Clause 26. The assembly of any of clauses 24-25, wherein the proximal part of the adhesive liner is configured to be coupled with the adhesive patch during shipping and storage.

Clause 27. The assembly of any of clauses 24-26, wherein the aperture in the distal part of the adhesive liner is configured to correspond with the shape of the sensor cap.

Clause 28. The assembly of any of clauses 24-27, wherein the adhesive patch further comprises an aperture, wherein the aperture of the distal part of the adhesive liner does not substantially overlap with the aperture of the adhesive patch.

Clause 29. The assembly of any of clauses 24-28, wherein the aperture of the distal part of the adhesive liner has a smaller diameter than the aperture of the adhesive patch.

Clause 30. An assembly for insertion of an analyte sensor, the assembly comprising:
(1) an applicator comprising:
   an applicator housing;
   a sharp;
   a carrier configured to releasably retain a sensor control device; and
   an applicator cap removably coupled with the applicator housing;
(2) the sensor control device configured to be worn on skin of a user, the sensor control device comprising:
a sensor control device housing;
sensor electronics disposed in the sensor control device housing;
the analyte sensor comprising:
a proximal portion coupled with the sensor electronics; and
a distal portion configured to be transcutaneously positioned through the skin of the user;
a sensor cap configured to cover the analyte sensor and the sharp, the sensor cap comprising a retention element, wherein the sensor cap is removably coupled with the sensor control device housing, and wherein the sensor cap is further configured such that the removal of the applicator cap further causes removal of the sensor cap from the sensor control device housing;
an adhesive patch comprising a distally-facing surface configured to adhere to the skin of the user; and
an adhesive liner removably coupled with the adhesive patch, the adhesive liner comprising an engagement element;
wherein the engagement element of the adhesive liner is configured to mate with the retention element of the sensor cap, and wherein the adhesive liner is configured such that removal of the sensor cap and the applicator cap causes removal of the adhesive liner from the adhesive patch.

Clause 31. The assembly of clause 30, wherein the analyte sensor is a glucose sensor.

Clause 32. The assembly of any of clauses 30-31, wherein the adhesive liner is configured to be coupled with the adhesive patch during shipping and storage.

Clause 33. The assembly of any of clauses 30-32, wherein the adhesive liner further comprises a split line to facilitate removal of the adhesive liner.

Clause 34. The assembly of any of clauses 30-33, wherein the split line is located in close proximity to the engagement element of the adhesive liner.

Clause 35. The assembly of any of clauses 30-34, wherein the adhesive liner is further configured to be peeled away in a clockwise direction.

Clause 36. The assembly of any of clauses 30-35, wherein the adhesive liner is further configured to be peeled away in a counterclockwise direction.

Clause 37. The assembly of any of clauses 30-36, wherein the engagement element of the adhesive liner comprises an aperture, wherein the aperture is configured to mate with the retention element of the sensor cap.

Clause 38. The assembly of any of clauses 30-37, wherein the retention element of the sensor cap comprises a protrusion, wherein the protrusion of the sensor cap is configured to mate with the engagement element of the adhesive liner.

Clause 39. An assembly for insertion of an analyte sensor, the assembly comprising:
(1) an applicator comprising:
   an applicator housing;
   a sharp;
   a carrier configured to releasably retain a sensor control device; and
   an applicator cap removably coupled with the applicator housing;
(2) the sensor control device configured to be worn on skin of a user, the sensor control device comprising:
a sensor control device housing;
sensor electronics disposed in the sensor control device housing;
the analyte sensor comprising:
a proximal portion coupled with the sensor electronics; and
a distal portion configured to be transcutaneously positioned through the skin of the user;
a sensor cap configured to cover the analyte sensor and the sharp, wherein the sensor cap is removably coupled with the sensor control device housing, and wherein the sensor cap is further configured to be removed such that the removal of the applicator cap further causes removal of the sensor cap from the sensor control device housing;
an adhesive patch comprising a distally-facing surface configured to adhere to the skin of the user; and
an adhesive liner removably coupled with the adhesive patch, the adhesive liner comprising a plurality of liner tabs, wherein a first liner tab of the plurality of liner tabs is coupled with the sensor cap at a first position, and a second liner tab of the plurality of liner tabs is coupled with the sensor cap at a second position, and wherein the adhesive liner is configured such that removal of the sensor cap and the applicator cap causes removal of the adhesive liner from the adhesive patch.

Clause 40. The assembly of clause 39, wherein the analyte sensor is a glucose sensor.

Clause 41. The assembly of any of clauses 39-40, wherein the adhesive liner is configured to be coupled with the adhesive patch during shipping and storage.

Clause 42. The assembly of any of clauses 39-41, wherein the adhesive liner further comprises a split line to facilitate removal of the adhesive liner.

Clause 43. The assembly of any of clauses 39-42, wherein the split line is located in close proximity to the first liner tab of the plurality of liner tabs.

Clause 44. The assembly of any of clauses 39-43, wherein the split line is located in close proximity to the second liner tab of the plurality of liner tabs.

Clause 45. The assembly of any of clauses 39-44, wherein the adhesive liner is further configured to be peeled away in a clockwise direction.

Clause 46. The assembly of any of clauses 39-45, wherein the adhesive liner is further configured to be peeled away in a counterclockwise direction.

Clause 47. The assembly of any of clauses 39-46, wherein the plurality of liner tabs comprises two liner tabs.

Clause 48. An assembly for insertion of an analyte sensor, the assembly comprising:
(1) an applicator comprising:
   an applicator housing;
   a sharp;
   a carrier configured to releasably retain a sensor control device; and
   an applicator cap removably coupled with the applicator housing;
(2) the sensor control device configured to be worn on skin of a user, the sensor control device comprising:
a sensor control device housing;
sensor electronics disposed in the sensor control device housing;
the analyte sensor comprising:
a proximal portion coupled with the sensor electronics; and
a distal portion configured to be transcutaneously positioned through the skin of the user;
a sensor cap configured to cover the analyte sensor and the sharp, the sensor cap comprising a ratchet feature, wherein the sensor cap is removably coupled with the sensor control device housing, and wherein the sensor cap is further configured such that the removal of the applicator cap further causes removal of the sensor cap from the sensor control device housing;
an adhesive patch comprising a distally-facing surface configured to adhere to the skin of the user; and
an adhesive liner removably coupled with the adhesive patch, the adhesive liner comprising a spiral pattern and a liner tab;
wherein the liner tab of the adhesive liner is configured to mate with the ratchet feature of the sensor cap, and wherein the adhesive liner is configured such that removal of the sensor cap and the applicator cap causes removal of the adhesive liner from the adhesive patch.

Clause 49. The assembly of clause 48, wherein the analyte sensor is a glucose sensor.

Clause 50. The assembly of any of clauses 48-49, wherein the adhesive liner is configured to be coupled with the adhesive patch during shipping and storage.

Clause 51. The assembly of any of clauses 48-50, wherein the adhesive liner is further configured to be peeled away in a clockwise direction.

Clause 52. The assembly of any of clauses 48-51, wherein the adhesive liner is further configured to be peeled away in a counterclockwise direction.

Clause 53. An assembly for insertion of an analyte sensor, the assembly comprising:
(1) an applicator comprising:
   an applicator housing;
   a sharp;
   a carrier configured to releasably retain a sensor control device; and
   an applicator cap removably coupled with the applicator housing;
(2) the sensor control device configured to be worn on skin of a user, the sensor control device comprising:
a sensor control device housing;
sensor electronics disposed in the sensor control device housing; and
the analyte sensor comprising:
a proximal portion coupled with the sensor electronics; and
a distal portion configured to be transcutaneously positioned through the skin of the user;
a sensor cap configured to cover the analyte sensor and the sharp, wherein the sensor cap is removably coupled with the sensor control device housing, and wherein the sensor cap is further configured such that the removal of the applicator cap further causes removal of the sensor cap from the sensor control device housing;
a collar configured to snap-fit into the sensor cap;
an adhesive patch comprising a distally-facing surface configured to adhere to the skin of the user; and
an adhesive liner removably coupled with the adhesive patch, the adhesive liner comprising an outer portion and a center portion;
wherein the center portion of the adhesive patch comprises a plurality of segments, wherein a plurality of the segments of the center portion of the adhesive liner are configured to mate with the collar individually, and wherein the adhesive liner is configured such that removal of the sensor cap and the applicator cap causes removal of the adhesive liner from the adhesive patch.

Clause 54. The assembly of clause 53, wherein the analyte sensor is a glucose sensor.

Clause 55. The assembly of any of clauses 53-54, wherein the adhesive liner is configured to be coupled with the adhesive patch during shipping and storage.

Clause 56. The assembly of any of clauses 53-55, wherein the adhesive liner is further configured to be peeled away in a clockwise direction.

Clause 57. The assembly of any of clauses 53-56, wherein the adhesive liner is further configured to be peeled away in a counterclockwise direction.

Clause 58. The assembly of any of clauses 53-57, wherein each of the plurality of segments of the center portion of the adhesive liner has a substantially triangular shape.

Clause 59. The assembly of any of clauses 53-58, wherein the outer portion of the adhesive liner further comprises a plurality of segments.

Clause 60. The assembly of any of clauses 53-59, wherein the collar comprises an elastomeric material.

Clause 61. The assembly of any of clauses 53-60, wherein the adhesive liner comprises siliconized paper.

Clause 62. The assembly of any of clauses 53-61, wherein the adhesive liner comprises siliconized polyester.

Clause 63. The assembly of any of clauses 53-62, wherein the adhesive liner further comprises a middle portion, separating the outer portion from the center portion.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the inventive scope of the claims by features, functions, steps, or elements that are not within that scope.

With respect to any of the applicator embodiments described herein, as well as any of the components thereof, including but not limited to the sharp, sharp module and sensor module embodiments, those of skill in the art will understand that said embodiments can be dimensioned and configured for use with sensors configured to sense an analyte level in a bodily fluid in the epidermis, dermis, or subcutaneous tissue of a subject. In some embodiments, for example, sharps and distal portions of analyte sensors disclosed herein can both be dimensioned and configured to be positioned at a particular end-depth (i.e., the furthest point of penetration in a tissue or layer of the subject's body, e.g., in the epidermis, dermis, or subcutaneous tissue). With respect to some applicator embodiments, those of skill in the art will appreciate that certain embodiments of sharps can be dimensioned and configured to be positioned at a different end-depth in the subject's body relative to the final end-depth of the analyte sensor. In some embodiments, for example, a sharp can be positioned at a first end-depth in the subject's epidermis prior to retraction, while a distal portion of an analyte sensor can be positioned at a second end-depth in the subject's dermis. In other embodiments, a sharp can be positioned at a first end-depth in the subject's dermis prior to retraction, while a distal portion of an analyte sensor can be positioned at a second end-depth in the subject's subcutaneous tissue. In still other embodiments, a sharp can be positioned at a first end-depth prior to retraction and the analyte sensor can be positioned at a second end-depth, wherein the first end-depth and second end-depths are both in the same layer or tissue of the subject's body.

Additionally, with respect to any of the applicator embodiments described herein, those of skill in the art will understand that an analyte sensor, as well as one or more structural components coupled thereto, including but not limited to one or more spring-mechanisms, can be disposed within the applicator in an off-center position relative to one or more axes of the applicator. In some applicator embodiments, for example, an analyte sensor and a spring mechanism can be disposed in a first off-center position relative to an axis of the applicator on a first side of the applicator, and the sensor electronics can be disposed in a second off-center position relative to the axis of the applicator on a second side of the applicator. In other applicator embodiments, the analyte sensor, spring mechanism, and sensor electronics can be disposed in an off-center position relative to an axis of the applicator on the same side. Those of skill in the art will appreciate that other permutations and configurations in which any or all of the analyte sensor, spring mechanism, sensor electronics, and other components of the applicator are disposed in a centered or off-centered position relative to one or more axes of the applicator are possible and fully within the scope of the present disclosure.

A number of deflectable structures are described herein, including but not limited to deflectable detent snaps 1402, deflectable locking arms 1412, sharp carrier lock arms 1524, sharp retention arms 1618, and module snaps 2202. These deflectable structures are composed of a resilient material such as plastic or metal (or others) and operate in a manner well known to those of ordinary skill in the art. The deflectable structures each has a resting state or position that the resilient material is biased towards. If a force is applied that causes the structure to deflect or move from this resting state or position, then the bias of the resilient material will cause the structure to return to the resting state or position once the force is removed (or lessened). In many instances these structures are configured as arms with detents, or snaps, but other structures or configurations can be used that retain the same characteristics of deflectability and ability to return to a resting position, including but not limited to a leg, a clip, a catch, an abutment on a deflectable member, and the like.

Additional details of suitable devices, systems, methods, components and the operation thereof along with related features are set forth in International Publication No. WO2018/136898 to Rao et. al., International Publication No. WO2019/236850 to Thomas et. al., International Publication No. WO2019/236859 to Thomas et. al., International Publication No. WO2019/236876 to Thomas et. al., and U.S. Patent Publication No. 2020/0196919, filed Jun. 6, 2019, each of which is incorporated by reference in its entirety herein. Further details regarding embodiments of applicators, their components, and variants thereof, are described in U.S. Patent Publication Nos. 2013/0150691, 2016/0331283, and 2018/0235520, all of which are incorporated by reference herein in their entireties and for all purposes. Further details regarding embodiments of sharp modules, sharps, their components, and variants thereof, are described in U.S. Patent Publication No. 2014/0171771, which is incorporated by reference herein in its entirety and for all purposes.

1-23. (canceled)

24. An assembly for insertion of an analyte sensor, the assembly comprising:
(1) an applicator comprising:
an applicator housing;
a sharp;
a carrier configured to releasably retain a sensor control device; and
an applicator cap removably coupled with the applicator housing;
(2) the sensor control device configured to be worn on skin of a user, the sensor control device comprising:
a sensor control device housing;
sensor electronics disposed in the sensor control device housing;
the analyte sensor comprising:
a proximal portion coupled with the sensor electronics; and
a distal portion configured to be transcutaneously positioned through the skin of the user;
a sensor cap configured to cover the analyte sensor and the sharp, wherein the sensor cap is removably coupled with the sensor control device housing, and wherein the sensor cap is further configured such that the removal of the applicator cap further causes removal of the sensor cap from the sensor control device housing;
an adhesive patch comprising a distally-facing surface configured to adhere to the skin of the user; and
an adhesive liner comprising:
a proximal part removably coupled with the adhesive patch; and
a distal part coupled with the sensor cap;
wherein the distal part of the adhesive liner comprises an aperture to receive the sensor cap, and wherein the adhesive liner is configured such that removal of the sensor cap and the applicator cap causes removal of the proximal part of the adhesive liner from the adhesive patch.

25. The assembly of claim 24, wherein the analyte sensor is a glucose sensor.

26. The assembly of claim 24, wherein the proximal part of the adhesive liner is configured to be coupled with the adhesive patch during shipping and storage.

27. The assembly of claim 24, wherein the aperture in the distal part of the adhesive liner is configured to correspond with the shape of the sensor cap.

28. The assembly of claim 24, wherein the adhesive patch further comprises an aperture, wherein the aperture of the distal part of the adhesive liner does not substantially overlap with the aperture of the adhesive patch.

29. The assembly of claim 28, wherein the aperture of the distal part of the adhesive liner has a smaller diameter than the aperture of the adhesive patch.

30-63. (canceled)

64. The assembly of claim 24, wherein the proximal part of the adhesive liner has a geometry that matches at least a portion of the shape of the adhesive patch.

65. The assembly of claim 24, wherein the aperture of the distal part of the adhesive liner is sized and shaped to receive the sensor cap in a non-rotational fit.

66. The assembly of claim 24, wherein the distal part of the adhesive liner is configured to be mechanically retained on the sensor cap.

67. The assembly of claim 24, wherein the aperture of the distal part of the adhesive liner is contoured to match a non-cylindrical profile of the sensor cap.

68. The assembly of claim 24, wherein the aperture in the distal part of the adhesive liner is non-circular and includes one or more shaped recesses or extensions to conform to the geometry of the sensor cap.

69. The assembly of claim 24, wherein the distal part of the adhesive liner is retained on the sensor cap by interference between an edge of the aperture and a corresponding ridge or protrusion on the sensor cap.

70. The assembly of claim 24, wherein the sensor cap includes a distal engagement region, and the distal part of the adhesive liner is shaped to seat against the engagement region during assembly.

71. The assembly of claim 24, wherein the distal part of the adhesive liner is configured to be captured around a step or shoulder of the sensor cap.

72. A method of inserting an analyte sensor, comprising:
providing an applicator comprising:
an applicator housing,
a sharp,
a carrier configured to releasably retain a sensor control device, and
an applicator cap removably coupled to the applicator housing;
providing the sensor control device, the sensor control device comprising:
a sensor control device housing,
sensor electronics disposed in the housing,
an analyte sensor having a proximal portion coupled with the sensor electronics and a distal portion configured to be transcutaneously positioned through the skin of a user,
a sensor cap configured to cover the analyte sensor and the sharp, the sensor cap being removably coupled with the sensor control device housing and configured to be removed upon removal of the applicator cap;
providing an adhesive patch comprising a distally-facing surface configured to adhere to the skin of the user;
providing an adhesive liner comprising:
a proximal part removably coupled with the adhesive patch, and
a distal part including an aperture coupled with the sensor cap;
positioning the adhesive patch and sensor control device on the skin of the user; and
removing the applicator cap and the sensor cap, wherein removal of the sensor cap and the applicator cap causes the distal part of the adhesive liner to pull the proximal part away from the adhesive patch, thereby removing the adhesive liner and exposing the adhesive surface of the patch.

73. The method of claim 72, wherein the analyte sensor is a glucose sensor.

74. The method of claim 72, wherein the proximal part of the adhesive liner is configured to be coupled with the adhesive patch during shipping and storage.

75. The method of claim 72, wherein the aperture in the distal part of the adhesive liner is configured to correspond with the shape of the sensor cap.

76. The method of claim 72, wherein the adhesive patch further comprises an aperture, wherein the aperture of the distal part of the adhesive liner does not substantially overlap with the aperture of the adhesive patch.

77. The method of claim 76, wherein the aperture of the distal part of the adhesive liner has a smaller diameter than the aperture of the adhesive patch.
